(19) United States
(12) Patent Application Publication
(10) Pub. No.: US 2025/0019369 A1
(43) Pub. Date: Jan. 16, 2025

(54) N-(4-aminocyclohexyl)pyrimidine-4-carboxamide derivatives as CD38 inhibitors

(71) Applicant: Cerevance, Inc., Boston, MA (US)

(72) Inventors: Roland Burli, Boston, MA (US); Kevin Doyle, Boston, MA (US)

(73) Assignee: Cerevance, Inc., Boston, MA (US)

(21) Appl. No.: 18/708,486

(22) PCT Filed: Nov. 9, 2022
(86) PCT No.: PCT/GB2022/052833

§ 371 Date: May 8, 2024

(30) Foreign Application Priority Data
Nov. 9, 2021 (GB) 2116077.5

(51) Int. Cl. C07D 403/14; A61K 31/4439; A61K 31/506; C07D 401/04; C07D 403/04; C07D 417/04

(52) U.S. Cl. CPC C07D 403/14; A61K 31/4439; A61K 31/506; C07D 401/04; C07D 403/04; C07D 417/04

(57) ABSTRACT

The present invention provides compounds of formula (I) and pharmaceutically acceptable salts, solvates and prodrugs thereof: wherein Het, X¹, X², L, R¹, R² and R³ are as defined in the specification, processes for their preparation, pharmaceutical compositions containing them and their use in therapy, particularly for use in treating disorders associated with CD38 activity.

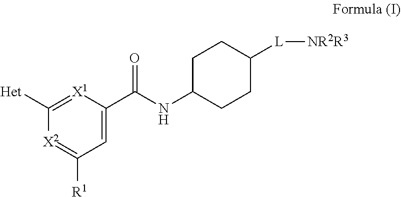

### FIELD OF THE INVENTION

The present invention relates to N-(4-aminocyclohexyl)pyrimidine-4-carboxamides and related compounds, processes for their preparation, pharmaceutical compositions containing them and their use in therapy, particularly for use in treating disorders associated with CD38 activity.

### BACKGROUND OF THE INVENTION

NAD⁺ homeostasis, aging & disease Nicotinamide adenine dinucleotide (NAD⁺) is an essential cellular component being extremely abundant in most living cells. NAD⁺ and its close analogue NADP⁺ perform similar redox functions within the cell, the latter being more confined to biosynthetic pathways and redox protective roles (Ying, 2008, Antioxid Redox Signal 10: 179). NAD⁺ and NADH (NAD(H)) are redox essential for a variety of electron-exchange-dependent biochemical reactions, particularly redox reactions involving oxidoreductase-mediated hydride transfer. Thus NAD(H) plays a vital role in the mitochondrial electron transport chain and cellular energy metabolism and as a co-enzyme linked to catabolism and harvesting of metabolic energy in all eukaryotic cells. However, the roles of NAD⁺ expand beyond its function as a co-enzyme, as NAD⁺ and its metabolites also act as degradation substrates for a wide range of enzymes, such as sirtuins (Hall et al, 2013, J Clin Invest 123: 973), SARM1 (Essuman et al, 2017, Neuron 93: 1334) and PARP enzymes (Murata et al, 2019, Mol Biol Cell 30: 2584). It is through these activities that NAD⁺ also links cellular metabolism to changes in signalling and transcriptional events and thus plays a central role in regulating cellular homeostasis and signalling.

NAD⁺ levels largely remain constant when used as a co-enzyme, but in non-redox reactions its levels are depleted from the cellular pool, thus requiring continuous re-synthesis and replenishment (Nikiforov et al, 2015, Crit Rev Biochem Mol Biol 50: 284). There are two main pathways for the synthesis of NAD⁺, the so called de novo pathway that utilizes the essential amino acid L-tryptophan to generate quinolinic acid (QA) that is further metabolized into NAD⁺ (Nikiforov et al, 2015, Crit Rev Biochem Mol Biol 50: 284), and the salvage pathway that utilizes nicotinamide (NAM), nicotinic acid (NA), and nicotinamide riboside (NR) (Imai & Yoshino, 2013, Diabetes Obes Metab Suppl. 3: 26). The salvage pathway is the main source of NAD⁺ in most cell types. NAD⁺ levels change during many physiological processes. Mounting evidence indicates that intracellular NAD⁺ levels are significantly affected by nutritional and environmental stimuli. These changes in NAD⁺ content are reflected into NAD⁺-dependent enzymatic activities, which in turn lead to changes in cellular metabolism, gene expression, and protein function. Therefore, maintenance of an optimal NAD⁺ concentration appears critical to maintain long term tissue homeostasis.

It has been clearly demonstrated that cellular NAD⁺ levels decline during chronological aging (Chini et al, 2017, Mol Cell Endocrinol 455: 62). This decline appears to play a crucial role in the development of metabolic dysfunction in aging and importantly, decline in cellular NAD⁺ levels has emerged as a potential key player in the pathogenesis of age-related conditions (Chini et al, 2017, Mol Cell Endocrinol 455: 62; Verdin, 2015, Science 350: 1208; Imai & Guarente, 2014, Trends Cell Biol 24: 464; Schultz & Sinclair, 2016, Cell Metab 23: 965); thus, maintaining NAD⁺ levels and subsequent cellular homeostasis may be a means of attenuating aging and age-related diseases such as Alzheimer's disease and Parkinson's disease (Chini et al, 2017, Mol Cell Endocrinol 455: 62). In support of this many studies have demonstrated that elevated NAD⁺ levels are associated with improved health and longer life span in multiple model organisms and humans (Fang et al, 2016, Cell Metab 24: 566; Fang et al, 2019, Nat Comms 10: 5284; Lehmann et al, 2017, Biol Open, 6: 141; Martens et al, 2018, Nat Comms 9: 1286; Mitchell et al, 2018, Cell Metab 27: 667; Covarrubias et al, 2021, Nat Rev Mol Cell Biol 22: 119; Perez et al, 2021, Mech Ag & Dev 197: 111499). As a result, there has been a growing interest in characterizing the role of NAD⁺ metabolism in age-related diseases and in developing pharmacological or nutraceutical interventions that increase NAD⁺ levels. In this regard, restoring NAD⁺ levels with NAD⁺ precursors, such as nicotinamide riboside (NR) and nicotinamide mononucleotide (NMN), has received some clinical interest (for reviews see Covarrubias et al, 2021, Nat Rev Mol Cell Biol 22: 119; Perez et al, 2021, Mech Ag & Dev 197: 111499) and inhibiting NAD⁺ consumption, e.g. by inhibiting CD38, has emerged as valuable therapeutic approach for age-related disorders and neurological diseases.

### CD38

Cluster of differentiation (CD38) is a multifunctional protein involved in i) cellular and tissue NAD⁺ homeostasis via its hydrolase function (Chini, 2009, Curr Pharm Des 15: 57) and ii) the generation of the second messengers ADPR and cyclic-ADPR (cADPR), via CD38s cyclase enzyme activity, that are subsequently involved in intracellular calcium signalling (Lee & Aarhus, 1991, Cell Regul 3: 203; Malavasi et al, 2008, Physiol Rev 88: 841). CD38 has a type II membrane orientation, with the catalytic site facing the outside of the cell (Chini, 2009, Curr Pharm Des 15: 57; Malavasi et al, 2008, Physiol Rev 88: 841). This was somewhat of a paradox given most substrates for NADase—CD38 are expected to be intracellular, however, it is now evident that CD38 degrades not only NAD⁺, but also circulating NAD⁺ precursors such as nicotinamide mononucleotide (NMN) and nicotinamide riboside (NR), before they can be incorporated into intracellular NAD⁺ biosynthetic pathways (Yoshino et al, 2018, Cell Metab 27: 513). Furthermore, CD38 has also been observed in intracellular membranes, such as in the nuclear membrane, mitochondria, and endoplasmic reticulum (Zhao et al, 2012, Sci Signal 5: ra67; Shrimp et al, 2014, J Am Chem Soc 136: 5656), a small fraction of CD38 is also expressed as a type III plasma membrane protein with the catalytic site facing the inside of the cell (Lui et al, 2017, Proc Natl Acad Sci USA. 114: 8283), and intra- and extracellular forms of CD38 have also been described (Chini, 2009, Curr Pharm Des 15: 57; Malavasi et al, 2008, Physiol Rev 88: 841). The relative roles and contributions of the different cellular pools of CD38 in the regulation of NAD⁺ homeostasis, calcium signalling, and subsequent cellular function is thus complex and still not clearly understood. It is apparent that CD38 is a very inefficient second messenger-generating enzyme, as it will hydrolyze almost a hundred molecules of NAD⁺ in order to generate one molecule of cADPR (Beers et al, 1995, J Clin Invest 95: 2385; Kim et al, 1993, Science 261: 1330), thus its role in NAD⁺ homeostasis may be its primary function reflected by its high substrate affinity and turnover rate compared to other NAD⁺ utilizing enzymes.

CD38 is expressed in the brain across species including mouse (Ceni et al, 2003, Biochem J 370: 175), rat (Yamada et al, 1997, Brain Res 756: 52; Braidy et al, 2014, Biogerontology 15: 177) and human (Mizuguchi et al, 1995, Brain Res 697: 235). In the human brain, it is interesting to note that CD38 is expressed in virtually all brain areas, with the highest expression levels in the caudate, pallidum, olfactory bulb, putamen, thalamus, and cingulate anterior (Quintana et al, 2019, Nat Comms 10: 668). Literature evidence suggests that CD38 is expressed in neurons (Yamada et al, 1997, Brain Res 756: 52; Mizuguchi et al, 1995, Brain Res 697: 235), astrocytes (Yamada et al, 1997, Brain Res 756: 52; Kou et al, 2009, J Neurosci Res 87: 2326), and microglial cells (Ma et al, 2012, Biochem Biophys Res Commun 418: 714; Mayo et al, 2008, J Immunol 181: 92). However, the applicant's in-house data suggest that CD38 expression predominates in the astrocytes of the human forebrain structures.

CD38 function is associated with effects on immunity, metabolic dysfunction, and behavioural deficits in mice (Barbosa et al, 2007, FASEB J 21: 3629; Lopatina et al, 2012, Front Neurosci 6: 182). Tissue NAD⁺ levels were found to be significantly higher in CD38-deficient mice suggesting that CD38 is the main NAD⁺ metabolising enzyme (NADase) in mammalian tissues. Concurrently it has been demonstrated that the expression and activity of CD38 increases with aging and that CD38 is at least in part the cause for the age-related NAD⁺ decline and subsequent mitochondrial dysfunction (Camacho-Pereira et al, 2016, Cell Metab 23: 1127). Furthermore, reduced NAD⁺ levels are a common observation among neurodegenerative diseases including Alzheimer's disease (Sonntag et al, 2017, Sci Rep 7: 14038), Parkinson's disease (Wakade et al, 2014, PLoS ONE 9: e109818), amyotrophic lateral sclerosis (Wang et al, 2017, Cell Rep 20: 2184), as well as multiple sclerosis (Braidy et al, 2013, Brain Res 1537: 267). Thus, attenuating CD38 activity, enhancing cellular levels of NAD⁺ and subsequent modulation of the diverse NAD⁺ related pathways could be a therapeutically viable approach to a range of brain and inflammatory disorders.

### Therapeutic Utility of CD38 Inhibitors

Several experimental data using CD38 knockout mice (KO) mice have demonstrated positive effects of CD38 deletion in models of neurodegeneration (Blacher et al, 2015, Ann Neurol 78: 88; Long et al, 2017, Neurochem Res 42: 283; Takaso et al, 2020, Sci Rep 10: 17795) and neuroinflammation (Choe et al, 2011, PLoS ONE 6: e19046; Raboon et al, 2019, Front Cell Neurosci 13: 258; for review see Guerreiro et al, 2020, Cells 9: 471), and a CD38 inhibitor molecule reversed age-related NAD⁺ decline and physiological effects of aging in mice (Tarrago et al, 2018, Cell Metab 27: 1081). Crossing of CD38 KO mouse with the APPswePS1DE9 model of Alzheimer's disease mouse model reduced amyloid plaque load and soluble Aβ levels, an effect that correlated with improved functional performance in a Morris water maze behavioural task (Blacher et al, 2015, Ann Neurol 78: 88).

In stroke models CD38-deficient mice showed decreased local expression of the proinflammatory cytokines and reduced ischemic injury and neurological deficits (Choe et al, 2011, PLoS ONE 6: e19046), whilst Long et al (Long et al, 2017, Neurochem Res 42: 283) showed an amelioration of histological and neurologic outcome following ischemic insult in CD38 KO mice. In models of multiple sclerosis, CD38 deficiency reduced severity of outcome in mouse experimental autoimmune encephalomyelitis (EAE) (Herrmann et al, 2016, Dis Mods Mechs 9: 1211) and suppressed neuroinflammation in a mouse model of demyelination (Raboon et al, 2019, Front Cell Neurosci 13: 258). Similarly, deletion of CD38 or supplementation of NAD⁺ attenuate axon degeneration in a mouse facial nerve axotomy model (Takaso et al, 2020, Sci Rep 10: 17795). Interestingly, a transcriptome-wide association study has identified CD38 as a potential susceptibility gene for Parkinson's disease (Yao et al, 2021, npj Parkinsons Dis 7: 79). In addition, CD38 KO mice are protected against obesity and metabolic syndrome (Barbosa et al, 2007, FASEB J 21: 3629; Chiang et al, 2015, PLoS ONE 10: e0134927) which are recognised risk factors for Alzheimer's disease. The regulatory impact of CD38 on the immune cells of the brain and periphery are also likely to be contributors to the beneficial impact of CD38 deletion or blockade on the various preclinical insult models (for reviews see Guerreiro et al, 2020, Cells 9: 471; Piedra-Quintero et al, 2020, Front Immunol 11: 597959) as neuroinflammation has been shown to be a major contributor across many of these diseases (Ransohoff, 2016, Science 353: 777).

Taken together, there is significant preclinical evidence to support the utility of augmenting cellular NAD⁺ levels by inhibiting its breakdown via blockade of CD38. The therapeutic utility in CNS diseases such as Alzheimer's disease, Parkinson's disease, multiple sclerosis, stroke and other neurodegenerative conditions will be reliant on achieving CNS penetration by the CD38 inhibitors. However, CD38 inhibitors will also likely have utility in other conditions such as autoimmune diseases, obesity and metabolic syndrome.

### Brain Permeability of Small Molecules

The central nervous system (CNS) is shielded from exposure to undesired substances by the blood-brain barrier (BBB). This restriction protects neurons from harmful interactions with toxins and other potentially harmful molecules. The BBB consists of brain capillary endothelial cells which have several unique attributes and functions: they have tight junctions leading to extremely low permeability via a paracellular route, they have low rates of endocytosis and, importantly, they highly express efflux transporter proteins with the specific function of recognizing and shuttling foreign substances out of the CNS (Gloor et al, 2001, Brain Res Rev 36: 258).

Importantly, the unbound drug concentration in the brain compartment is a critical parameter that needs to be considered when evaluating the suitability of molecules as potential therapies for neurological and neurodegenerative disorders: it is generally accepted that only the unbound fraction of drug may be available for occupying the desired target in order to exert a pharmacological effect.

There is a need for treatment of the above diseases and conditions and others described herein with compounds that are CD38 inhibitors. The present invention provides such CD38 inhibitors, including brain permeable CD38 inhibitors.

### SUMMARY OF THE INVENTION

A first aspect of the present invention provides a compound of formula (I):

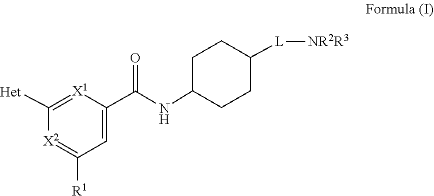

or a pharmaceutically acceptable salt, solvate or prodrug thereof, wherein:
Het is a 5-membered heteroaryl group comprising two heteroatoms independently selected from N and S, wherein the 5-membered heteroaryl group may optionally be substituted with one or two substituents independently selected from C₁-C₃ alkyl, C₁-C₃ fluoroalkyl and C₁-C₃ hydroxyalkyl;
X¹ is CH or N, and X² is CH or N, wherein at least one of X¹ and X² is N;
L is a bond, CH₂, CHMe, CMe₂ or CO;
R¹ is C₁-C₄ alkyl, C₃-C₆ cycloalkyl, hydroxyl, —O—(C₁-C₄ alkyl), or —O—(C₃-C₆ cycloalkyl), each of which may optionally be fluoro-substituted;
R² is hydrogen, C₁-C₄ alkyl, C₁-C₄ fluoroalkyl, —CHO, —CO—(C₁-C₃ alkyl) or —CO—(C₁-C₃ fluoroalkyl);
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

The first aspect of the present invention also provides a compound of formula (I):

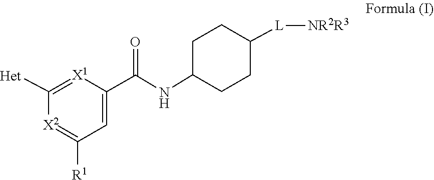

or a pharmaceutically acceptable salt, solvate or prodrug thereof, wherein:
Het is a 5-membered heteroaryl group comprising two heteroatoms independently selected from N and S, wherein the 5-membered heteroaryl group may optionally be substituted with one or two substituents independently selected from C₁-C₃ alkyl, C₁-C₃ fluoroalkyl and C₁-C₃ hydroxyalkyl;
X¹ is CH or N, and X² is CH or N, wherein at least one of X¹ and X² is N;
L is a bond, CH₂, CHMe, CMe₂ or CO;
R¹ is C₁-C₄ alkyl, C₃-C₆ cycloalkyl, hydroxyl, —O—(C₁-C₄ alkyl), or —O—(C₃-C₆ cycloalkyl), each of which may optionally be fluoro-substituted;
R² is C₁-C₃ alkyl, C₁-C₃ fluoroalkyl, —CHO, —CO—(C₁-C₃ alkyl) or —CO—(C₁-C₃ fluoroalkyl);
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

In one embodiment of the compounds of formula (I), Het is an imidazolyl, pyrazolyl, thiazolyl or isothiazolyl group, each of which may optionally be substituted with one or two substituents independently selected from C₁-C₃ alkyl, C₁-C₃ fluoroalkyl and C₁-C₃ hydroxyalkyl. In one embodiment, Het is an imidazolyl, pyrazolyl, thiazolyl or isothiazolyl group, each of which may optionally be substituted with one substituent independently selected from C₁-C₃ alkyl and C₁-C₃ fluoroalkyl. In one embodiment, Het is an imidazolyl, pyrazolyl or thiazolyl group, each of which may optionally be substituted with one substituent independently selected from methyl or ethyl. In one embodiment, Het is an imidazol-1-yl, 1-methyl-imidazol-5-yl, pyrazol-4-yl, or thiazol-5-yl group. In a preferred embodiment, Het is an imidazol-1-yl, 1-methyl-imidazol-5-yl, or thiazol-5-yl group.

In one embodiment of the compounds of formula (I), X¹ is N and X² is CH. In another embodiment, X¹ is CH and X² is N. In a preferred embodiment, X¹ is N and X² is N.

In one embodiment of the compounds of formula (I), L is a bond, CH₂ or CO. In a preferred embodiment, L is a bond.

In one embodiment of the compounds of formula (I), R¹ is C₁-C₄ alkyl or C₃-C₆ cycloalkyl, each of which may optionally be fluoro-substituted. In one embodiment, R¹ is C₁-C₄ alkyl or C₃-C₄ cycloalkyl, each of which may optionally be fluoro-substituted. In one embodiment, R¹ is C₁-C₃ alkyl or cyclopropyl, each of which may optionally be fluoro-substituted. In one embodiment, R¹ is methyl, ethyl, n-propyl, iso-propyl or cyclopropyl. In a preferred embodiment, R¹ is methyl, ethyl or cyclopropyl.

In one embodiment of the compounds of formula (I), R² is C₁-C₃ alkyl or C₁-C₃ fluoroalkyl. In one embodiment, R² is C₁-C₃ fluoroalkyl. In one embodiment, R² is C₁-C₂ fluoroalkyl. In a preferred embodiment, R² is —CH₂CF₃, —CH₂CHF₂ or —CH₂CH₂F. In another preferred embodiment, R² is —CH₂CF₃ or —CH₂CHF₂.

In one embodiment of the compounds of formula (I), R² and R³, together with the nitrogen to which they are attached, form an azetidin-1-yl, pyrrolidin-1-yl or piperidin-1-yl group, each of which may optionally be substituted with one, two, three or four fluoro substituents. In one embodiment, R² and R³, together with the nitrogen to which they are attached, form a pyrrolidin-1-yl group, which may optionally be substituted with one, two, three or four fluoro substituents. In a preferred embodiment, R² and R³, together with the nitrogen to which they are attached, form a pyrrolidin-1-yl group, which is substituted with one, two or three fluoro substituents. In another preferred embodiment, R² and R³, together with the nitrogen to which they are attached, form a pyrrolidin-1-yl group, which is substituted with two or three fluoro substituents.

In a preferred embodiment of the compounds of formula (I), the two substituents on the cyclohexyl group (—NH— and -L-) are trans to each other.

The first aspect of the present invention further provides a compound of formula (II):

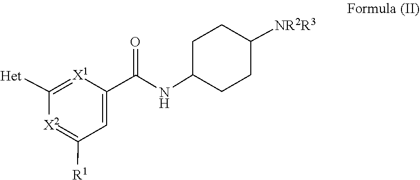

or a pharmaceutically acceptable salt, solvate or prodrug thereof, wherein:
Het is a 5-membered heteroaryl group comprising two heteroatoms independently selected from N and S, wherein the 5-membered heteroaryl group may optionally be substituted with one or two substituents independently selected from C₁-C₃ alkyl, C₁-C₃ fluoroalkyl and C₁-C₃ hydroxyalkyl;
X¹ is CH or N, and X² is CH or N, wherein at least one of X¹ and X² is N;
R¹ is C₁-C₄ alkyl, C₃-C₆ cycloalkyl, hydroxyl, —O—(C₁-C₄ alkyl), or —O—(C₃-C₆ cycloalkyl), each of which may optionally be fluoro-substituted;
R² is C₁-C₃ alkyl or C₁-C₃ fluoroalkyl;
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

In one embodiment of the compounds of formula (II), Het is an imidazolyl, pyrazolyl, thiazolyl or isothiazolyl group, each of which may optionally be substituted with one or two substituents independently selected from C₁-C₃ alkyl, C₁-C₃ fluoroalkyl and C₁-C₃ hydroxyalkyl. In one embodiment, Het is an imidazolyl, pyrazolyl, thiazolyl or isothiazolyl group, each of which may optionally be substituted with one substituent independently selected from C₁-C₃ alkyl and C₁-C₃ fluoroalkyl. In one embodiment, Het is an imidazolyl, pyrazolyl or thiazolyl group, each of which may optionally be substituted with one substituent independently selected from methyl or ethyl. In one embodiment, Het is an imidazol-1-yl, 1-methyl-imidazol-5-yl, pyrazol-4-yl, or thiazol-5-yl group. In a preferred embodiment, Het is an imidazol-1-yl, 1-methyl-imidazol-5-yl, or thiazol-5-yl group.

In one embodiment of the compounds of formula (II), X¹ is N and X² is CH. In another embodiment, X¹ is CH and X² is N. In a preferred embodiment, X¹ is N and X² is N.

In one embodiment of the compounds of formula (II), R¹ is C₁-C₄ alkyl or C₃-C₆ cycloalkyl, each of which may optionally be fluoro-substituted. In one embodiment, R¹ is C₁-C₄ alkyl or C₃-C₄ cycloalkyl, each of which may optionally be fluoro-substituted. In one embodiment, R¹ is C₁-C₃ alkyl or cyclopropyl, each of which may optionally be fluoro-substituted. In one embodiment, R¹ is methyl, ethyl, n-propyl, iso-propyl or cyclopropyl. In a preferred embodiment, R¹ is methyl, ethyl or cyclopropyl.

In one embodiment of the compounds of formula (II), R² is C₁-C₃ fluoroalkyl. In one embodiment, R² is C₁-C₂ fluoroalkyl. In a preferred embodiment, R² is —CH₂CF₃, —CH₂CHF₂ or —CH₂CH₂F. In another preferred embodiment, R² is —CH₂CF₃ or —CH₂CHF₂.

In one embodiment of the compounds of formula (II), R² and R³, together with the nitrogen to which they are attached, form an azetidin-1-yl, pyrrolidin-1-yl or piperidin-1-yl group, each of which may optionally be substituted with one, two, three or four fluoro substituents. In one embodiment, R² and R³, together with the nitrogen to which they are attached, form a pyrrolidin-1-yl group, which may optionally be substituted with one, two, three or four fluoro substituents. In a preferred embodiment, R² and R³, together with the nitrogen to which they are attached, form a pyrrolidin-1-yl group, which is substituted with one, two or three fluoro substituents. In another preferred embodiment, R² and R³, together with the nitrogen to which they are attached, form a pyrrolidin-1-yl group, which is substituted with two or three fluoro substituents.

In a preferred embodiment of the compounds of formula (II), the two substituents on the cyclohexyl group (—NH— and —NR²R³) are trans to each other.

The first aspect of the present invention further provides a compound of formula (III):

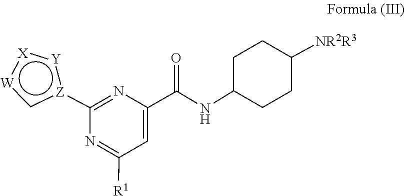

or a pharmaceutically acceptable salt, solvate or prodrug thereof, wherein:
each W, X, Y and Z is independently CH, CMe, N, NH, NMe or S, wherein two of W, X, Y and Z are CH or CMe, and the other two of W, X, Y and Z are N, NH, NMe or S;
R¹ is C₁-C₄ alkyl or C₃-C₄ cycloalkyl;
R² is C₁-C₃ alkyl or C₁-C₃ fluoroalkyl;
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

For the avoidance of doubt, it is noted that the group

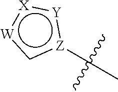

is a 5-membered heteroaryl group comprising two heteroatoms independently selected from N and S, wherein the 5-membered heteroaryl group may optionally be substituted with one or two methyl groups.

In one embodiment of the compounds of formula (III), W is CH, N or NH; X is CH, N or NH; Y is CH, N, NH, NMe or S; and Z is C or N. In one embodiment, W is CH or N; X is CH, N or NH; Y is CH, NMe or S; and Z is C or N. In one embodiment, the group

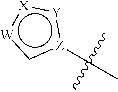

is an imidazol-1-yl, 1-methyl-imidazol-5-yl, pyrazol-4-yl, or thiazol-5-yl group. In a preferred embodiment, the group

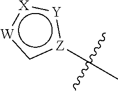

is an imidazol-1-yl, 1-methyl-imidazol-5-yl, or thiazol-5-yl group.

In one embodiment of the compounds of formula (III), R¹ is C₁-C₃ alkyl or cyclopropyl.

In one embodiment, R¹ is methyl, ethyl, n-propyl, iso-propyl or cyclopropyl. In a preferred embodiment, R¹ is methyl, ethyl or cyclopropyl.

In one embodiment of the compounds of formula (III), R² is C₁-C₃ fluoroalkyl. In one embodiment, R² is C₁-C₂ fluoroalkyl. In a preferred embodiment, R² is —CH₂CF₃, —CH₂CHF₂ or —CH₂CH₂F. In another preferred embodiment, R² is —CH₂CF₃ or —CH₂CHF₂.

In one embodiment of the compounds of formula (III), R² and R³, together with the nitrogen to which they are attached, form an azetidin-1-yl, pyrrolidin-1-yl or piperidin-1-yl group, each of which may optionally be substituted with one, two, three or four fluoro substituents. In one embodiment, R² and R³, together with the nitrogen to which they are attached, form a pyrrolidin-1-yl group, which may optionally be substituted with one, two, three or four fluoro substituents. In a preferred embodiment, R² and R³, together with the nitrogen to which they are attached, form a pyrrolidin-1-yl group, which is substituted with one, two or three fluoro substituents. In another preferred embodiment, R² and R³, together with the nitrogen to which they are attached, form a pyrrolidin-1-yl group, which is substituted with two or three fluoro substituents.

In a preferred embodiment of the compounds of formula (III), the two substituents on the cyclohexyl group (—NH— and —NR²R³) are trans to each other.

A second aspect of the present invention provides a compound selected from:
2-(1H-imidazol-1-yl)-6-methyl-N-((1r,4r)-4-((2,2,2-trifluoroethyl)amino)cyclohexyl)pyrimidine-4-carboxamide;
N-((1r,4r)-4-((2,2-difluoroethyl)amino)cyclohexyl)-2-(1H-imidazol-1-yl)-6-methyl-pyrimidine-4-carboxamide;
6-cyclopropyl-N-((1r,4r)-4-((2,2-difluoroethyl)amino)cyclohexyl)-2-(1H-imidazol-1-yl)pyrimidine-4-carboxamide;
6-methyl-2-(1-methyl-1H-imidazol-5-yl)-N-((1r,4r)-4-((2,2,2-trifluoroethyl)amino)cyclohexyl)pyrimidine-4-carboxamide;
6-methyl-2-(thiazol-5-yl)-N-((1r,4r)-4-((2,2,2-trifluoroethyl)amino)cyclohexyl)pyrimidine-4-carboxamide;
6-ethyl-2-(1H-imidazol-1-yl)-N-((1r,4r)-4-((2,2,2-trifluoroethyl)amino)cyclohexyl)pyrimidine-4-carboxamide;
2-(1H-imidazol-1-yl)-6-methyl-N-((1r,4r)-4-(methyl(2,2,2-trifluoroethyl)amino)cyclohexyl)pyrimidine-4-carboxamide;
N-((1r,4r)-4-((2,2-difluoroethyl)amino)cyclohexyl)-6-methyl-2-(thiazol-5-yl)pyrimidine-4-carboxamide;
N-((1r,4r)-4-((2-fluoroethyl)amino)cyclohexyl)-2-(1H-imidazol-1-yl)-6-methyl-pyrimidine-4-carboxamide;
N-((1r,4r)-4-(3,3-difluoropyrrolidin-1-yl)cyclohexyl)-2-(1H-imidazol-1-yl)-6-methyl-pyrimidine-4-carboxamide;
N-((1s,4s)-4-(3,3-difluoropyrrolidin-1-yl)cyclohexyl)-2-(1H-imidazol-1-yl)-6-methyl-pyrimidine-4-carboxamide;
N-((1s,4r)-4-((S)-3-fluoropyrrolidin-1-yl)cyclohexyl)-2-(1H-imidazol-1-yl)-6-methyl-pyrimidine-4-carboxamide;
N-((1r,4r)-4-((R)-3-fluoropyrrolidin-1-yl)cyclohexyl)-2-(1H-imidazol-1-yl)-6-methyl-pyrimidine-4-carboxamide;
6-methyl-2-(1H-pyrazol-4-yl)-N-((1r,4r)-4-((2,2,2-trifluoroethyl)amino)cyclohexyl)pyrimidine-4-carboxamide;
N-((1r,4r)-4-((2,2-difluoroethyl)amino)cyclohexyl)-6-methyl-2-(1H-pyrazol-4-yl)pyrimidine-4-carboxamide;
4-cyclopropyl-N-((1r,4r)-4-((2,2-difluoroethyl)amino)cyclohexyl)-6-(1H-imidazol-1-yl)picolinamide;
N-((1r,4r)-4-(ethylamino)cyclohexyl)-2-(1H-imidazol-1-yl)-6-methyl-pyrimidine-4-carboxamide;
4-cyclopropyl-6-(1H-imidazol-1-yl)-N-((1r,4r)-4-((2,2,2-trifluoroethyl)amino)cyclohexyl)picolinamide;
2-(1H-imidazol-1-yl)-6-methyl-N-((1r,4r)-4-((1,1,1-trifluoro-2-methylpropan-2-yl)amino)cyclohexyl)pyrimidine-4-carboxamide;
2-(1H-imidazol-1-yl)-6-methyl-N-((1S,4r)-4-(((S)-1,1,1-trifluoropropan-2-yl)amino)cyclohexyl)pyrimidine-4-carboxamide;
2-(1H-imidazol-1-yl)-6-methyl-N-((1R,4r)-4-(((R)-1,1,1-trifluoropropan-2-yl)amino)cyclohexyl)pyrimidine-4-carboxamide;
6-methyl-2-(5-methyl-1H-imidazol-1-yl)-N-((1r,4r)-4-((2,2,2-trifluoroethyl)amino)cyclohexyl)pyrimidine-4-carboxamide;
2-(1H-imidazol-1-yl)-6-methyl-N-((1r,4r)-4-(((2,2,2-trifluoroethyl)amino)methyl)cyclohexyl)pyrimidine-4-carboxamide;
2-(1H-imidazol-1-yl)-6-methyl-N-((1r,4r)-4-((2,2,2-trifluoroethyl)carbamoyl)cyclohexyl)pyrimidine-4-carboxamide;
N-((1r,4r)-4-aminocyclohexyl)-2-(1H-imidazol-1-yl)-6-methyl-pyrimidine-4-carboxamide;
2-(1H-imidazol-1-yl)-6-methyl-N-((1r,4r)-4-((2,2,2-trifluoroethyl-1,1-d₂)amino)cyclohexyl)pyrimidine-4-carboxamide;
   or an enantiomer of any of the foregoing;
   or a pharmaceutically acceptable salt, solvate or prodrug of any of the foregoing.

Preferably the compound of the first or second aspect has a chemical purity of 95% or more, preferably 96% or more, preferably 97% or more, preferably 98% or more, preferably 99% or more, preferably 99.5% or more, preferably 99.8% or more, preferably 99.9% or more, as measured by HPLC or UPLC.

Preferably the compound of the first or second aspect has a stereochemical purity of 95% or more, preferably 96% or more, preferably 97% or more, preferably 98% or more, preferably 99% or more, preferably 99.5% or more, preferably 99.8% or more, preferably 99.9% or more, as measured by XRPD or SFC.

A third aspect of the present invention provides a process for the preparation of a compound, salt, solvate or prodrug according to the first or second aspect of the present invention, wherein the process comprises the step of reacting a compound of formula (IV) with an amine of formula (V):

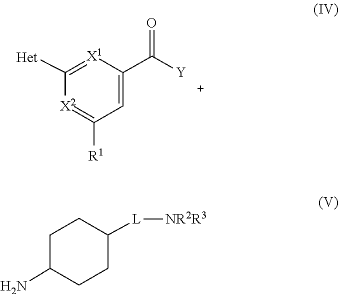

wherein Het, X¹, X², L, R¹, R² and R³ are as defined in the first or second aspect of the present invention; Y is —OH, —OR⁴, —O—CO—R⁴ or —Cl; and R⁴ is C₁-C₃ alkyl; and optionally thereafter carrying out one or more of the following procedures:
converting a compound of formula (I), (II) or (III) into another compound of formula (I), (II) or (III);
removing any protecting groups;
forming a pharmaceutically acceptable salt.

When Y is —OH, the compound of formula (IV) is a carboxylic acid (IVA). When Y is —OR⁴, the compound of formula (IV) is an ester (IVB). When Y is —O—CO—R⁴, the compound of formula (IV) is an anhydride (IVC). When Y is —Cl, the compound of formula (IV) is an acid chloride (IVD).

The step of reacting a carboxylic acid (IVA) with an amine of formula (V) may be carried out in the presence of a coupling agent, such as HATU or T₃P, and a base, such as DIPEA or TEA. Typically, DMF, NMP or DCM is used as a solvent, although other polar aprotic solvents can also be used. Typically, the reaction is carried out at about 20-50° C. (typically about 25° C.) and takes about 0.5-5 hours (typically about 1-2 hours).

The step of reacting an ester (IVB) with an amine of formula (V) may be carried out in the presence of trimethylaluminum in a non-polar solvent, such as toluene. Typically, the reaction is carried out at about 70-100° C. (typically about 90° C.) and takes about 0.5-5 hours (typically about 1-2 hours).

The step of reacting an anhydride (IVC) with an amine of formula (V) may be carried out in the presence of a base, such as DIPEA or TEA. Typically, DMF, NMP or DCM is used as a solvent, although other polar aprotic solvents can also be used. Typically, the reaction is carried out at about 20-50° C. (typically about 25° C.) and takes about 0.5-5 hours (typically about 1-2 hours).

Carboxylic acids of formula (IVA) and esters of formula (IVB) may be prepared as shown in Scheme 1.

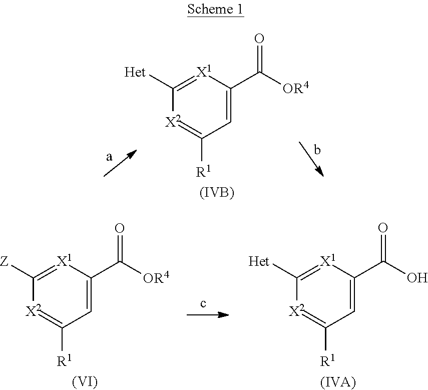

In step (a), ester (VI), wherein Z is a leaving group such as chlorine, is reacted with a heteroaryl compound, such as imidazole, typically in the presence of a base, such as DIPEA or TEA, to provide ester (IVB). Typically, DMF is used as a solvent. Typically, the reaction is carried out at about 90-110° C. (typically about 100° C.) and takes about 2-24 hours (typically about 4-12 hours). The reaction may be carried out under an atmosphere of nitrogen.

In step (b), the ester (IVB) is converted into the carboxylic acid (IVA) by treatment with a base, such as LiOH. Typically, THF and water are used as solvent. Typically, the reaction is carried out at about 20-50° C. (typically about 25° C.) and takes about 0.5-5 hours (typically about 1-2 hours).

Alternatively, carboxylic acid (IVA) may be prepared from ester (VI) in a one-step process. In step (c), ester (VI) is reacted with a heteroaryl compound activated with, for example, a 4,4,5,5-tetramethyl-1,3,2-dioxaborolan-2-yl group, such as 1-methyl-5-(4,4,5,5-tetramethyl-1,3,2-dioxaborolan-2-yl)imidazole, 5-(4,4,5,5-tetramethyl-1,3,2-dioxaborolan-2-yl)thiazole or 1-tetrahydropyran-2-yl-4-(4,4,5,5-tetramethyl-1,3,2-dioxaborolan-2-yl)pyrazole, to directly provide carboxylic acid (IVA). The ester (VI) and the activated heteroaryl compound may be reacted in the presence of Cs₂CO₃ and Pd(dppf)Cl₂. Typically, dioxane and water are used as solvent. Typically, the reaction is carried out at about 80-100° C. (typically about 90° C.) and takes about 0.5-5 hours (typically about 1-2 hours). Typically the reaction is carried out under an atmosphere of nitrogen.

When X¹ is N and X² is CH, the methyl ester of formula (IVB′) and the carboxylic acid of formula (IVA′) may be prepared as shown in Scheme 2.

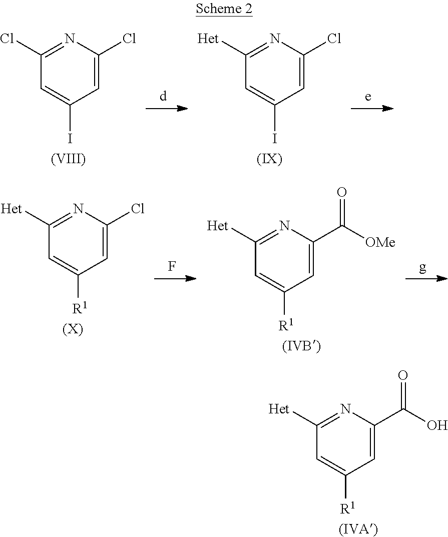

In step (d), 2,6-dichloro-4-iodo-pyridine (VIII) is treated with a heteroaryl salt, such as imidazole sodium, to provide compound (IX). Typically, THF is used as a solvent. Typically, the reaction is carried out at about 50-70° C. (typically about 60° C.) and takes about 6-24 hours (typically about 12 hours). The required heteroaryl salt may be prepared by treating a heteroaryl compound, such as imidazole, with a base such as NaH; typically THF is used as a solvent; typically the reaction is carried out at about 0° C. and takes about 0.1-2 hours (typically about 0.5 hour). In step (e), compound (IX) may be reacted with boronic acid R¹—B(OH)₂, typically in the presence of K₃PO₄, PCy₃·BF₄ and Pd(OAc)₂ to provide compound (X). Typically, water and toluene are used as solvent. Typically, the reaction is carried out at about 100-120° C. (typically about 110° C.) and takes about 3-12 hours (typically about 6 hours). Typically the reaction is carried out under an atmosphere of nitrogen. In step (f), compound (X) may be treated with carbon monoxide and methanol, typically in the presence of TEA and Pd(dppf)Cl₂ to provide methyl ester (IVB′). Typically, the reaction is carried out at about 70-90° C. (typically about 80° C.) and takes about 12-24 hours (typically about 16 hours). In step (g), methyl ester (IVB′) is converted into carboxylic acid (IVA′) by treatment with a base, such as LiOH. Typically, THF and water are used as solvent. Typically, the reaction is carried out at about 20-30° C. (typically about 25° C.) and takes about 0.5-5 hours (typically about 1-2 hours).

An example of converting a compound of formula (I), (II) or (III) into another compound of formula (I), (II) or (III) can be found in Example 28. The step of converting a compound of formula (I), (II) or (III) into another compound of formula (I), (II) or (III) may be carried out by an alkylation reaction, wherein R² which is hydrogen is replaced by another R² group, or R³ which is hydrogen is replaced by another R³ group. Typically, in the alkylation reaction a compound of formula (I), (II) or (III) wherein R² or R³ is hydrogen may be combined with a compound LG-R² or LG-R³, wherein LG is a leaving group such as halo (such as fluoro, chloro, bromo, or iodo), a sulfate group (such as methyl sulfate), or a sulfonate group (such as mesylate, triflate, or tosylate), and R² and R³ are as defined in the first or second aspect of the present invention. Typically, the reaction is carried out in the presence of a base such as K₂CO₃, typically in the further presence of KI and 1,4,7,10,13,16-hexaoxacyclooctadecane. The reaction is typically carried out in a solvent such as DMF at about 80-100° C. (typically about 90° C.) and takes about 1 hour.

It will be appreciated by those skilled in the art that in the processes of the present invention certain functional groups such as phenol, hydroxy or amino groups in the reagents may need to be protected by protecting groups. Thus, the preparation of the compounds, salts, solvates and prodrugs of the present invention may involve, at an appropriate stage, the introduction and/or removal of one or more protecting groups. An example of the introduction and/or removal of one or more protecting groups can be found in Example 28.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

The compounds of the first and second aspect of the present invention may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of the first and second aspect are in the form of a hydrochloride, formate or fumarate salt. Examples of pharmaceutically acceptable salts of the compounds of the first and second aspect of the present invention may be found in Examples 9, 19 and 27.

A salt of a compound of the first or second aspect of the present invention may also be formed between a protic acid functionality of a compound of the first or second aspect and a suitable cation. Suitable cations include, but are not limited to lithium, sodium, potassium, magnesium, calcium and ammonium. In one embodiment of the invention, the salt is a sodium or potassium salt.

Compounds of the first and second aspect of the present invention and their salts may be in the form of hydrates or solvates which form another embodiment of the present invention. Such solvates may be formed with common organic solvents including, but not limited to alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of the first or second aspect. Generally, the prodrugs are pharmacologically inert chemical derivatives that can be converted in vivo to the active drug molecules to exert a therapeutic effect. Any of the compounds of the first and second aspect of the present invention can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

Where the compounds, salts, solvates and prodrugs of the present invention are capable of existing in stereoisomeric forms, it will be understood that the invention encompasses the use of all geometric and optical isomers (including atropisomers) and mixtures thereof. The use of tautomers and mixtures thereof also forms an embodiment of the present invention. The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, more typically less than 1%, and most typically less than 0.5% by weight. Enantiomerically pure isomers are particularly desired.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and 131I.

In one embodiment, the compounds, salts, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, solvates and prodrugs of the present invention can be isotopically labelled, for example, any of examples 1-16 or 19-28.

In one embodiment, the compounds, salts, solvates and prodrugs of the present invention may bear one or more radiolabels. Such radiolabels may be introduced by using radiolabel-containing reagents in the synthesis of the compounds, salts, solvates or prodrugs, or may be introduced by coupling the compounds, salts, solvates or prodrugs to chelating moieties capable of binding to a radioactive metal atom. Such radiolabelled versions of compounds, salts, solvates and prodrugs may be used, for example, in diagnostic imaging studies.

In one embodiment, the compounds, salts, solvates and prodrugs of the present invention may be tritiated, i.e. they contain one or more 3H (T) atoms. Any of the compounds, salts, solvates and prodrugs of the present invention can be tritiated, for example, any of examples 1-16 or 19-28.

The compounds, salts, solvates and prodrugs of the present invention may be amorphous or in a polymorphic form or a mixture of any of these, each of which is an embodiment of the present invention.

The compounds, salts, solvates and prodrugs of the present invention have activity as pharmaceuticals and may be used in treating or preventing a disease, disorder or condition associated with CD38 activity. Diseases, disorders and conditions associated with CD38 activity include:
CNS diseases and diseases requiring treatment via the CNS, including Parkinson's disease (Camacho-Pereira et al, 2016, Cell Metab 23: 1127; Perez et al, 2021, Mech Ag & Dev 197: 111499; Wakade et al, 2014, PLoS ONE 9: e109818; Yao et al, 2021, npj Parkinsons Dis 7: 79); Alzheimer's disease (Blacher et al, 2015, Ann Neurol 78: 88; Sonntag et al, 2017, Sci Rep 7: 14038); frontotemporal dementia; progressive supranuclear palsy (PSP); tauopathies; other non-Alzheimer's dementias; stroke and ischemic insults (Choe et al, 2011, PLoS ONE 6: e19046); traumatic brain injury (TBI) (Long et al, 2017, Neurochem Res 42: 283; Takaso et al, 2020, Sci Rep 10: 17795); multiple sclerosis (Herrmann et al, 2016, Dis Mods Mechs 9: 1211; Raboon et al, 2019, Front Cell Neurosci 13: 258); autoimmune diseases with associated neuronal damage such as Muckle-Wells syndrome; motor neuron disease such as amyotrophic lateral sclerosis (ALS) (Wang et al, 2017, Cell Rep 20: 2184); axonal neuropathy and axonal degeneration such as diabetic neuropathy (Lin et al, 2016, Cell Rep 17: 69); Wallerian degeneration (Essuman et al, 2017, Neuron 93: 1334; Takaso et al, 2020, Sci Rep 10: 17795; Krauss et al, 2020, TiPS 41: 281); ataxia telangiectasia, Friedreich's ataxia and other ataxias such as spinocerebellar ataxia 7 (SCA7) (Fang et al, 2016, Cell Metab 24: 566);
aging and senescence (Chini et al, 2017, Mol Cell Endocrinol 455: 62; Verdin, 2015, Science 350: 1208; Imai & Guarente, 2014, Trends Cell Biol 24: 464; Schultz & Sinclair, 2016, Cell Metab 23: 965);
neuroinflammation (Choe et al, 2011, PLoS ONE 6: e19046; Raboon et al, 2019, Front Cell Neurosci 13: 258; Guerreiro et al, 2020, Cells 9: 471; Najjar et al, 2013, J Neuroinflamm 10: 43);
depression, schizophrenia, anxiety, stress and post-traumatic stress disorder (PTSD) (Tabak et al, 2016, Clin Psychol Sci 4: 17);
glaucoma and age-related macular degeneration (AMD) (Cimaglia et al, 2020, Nutrients 12: 2871; Jadeja et al, 2020, Oxidative Medicine and Cellular Longevity article 2692794);
hearing loss (Brown et al, 2014, Cell Metab 20: 1059; Nakanishi et al, 2020, Frontiers in Neurology ii: article 141; Okur et al, 2020, npj Aging and Mechanisms of Disease 6: 1);
autoimmune diseases such as rheumatoid arthritis (RA) and Lupus (Cole et al, 2018, Arthritis Research & Therapy 20: 85; Garcia-Rodriguez et al, 2018, Scientific Reports 8: 3357);
obesity and metabolic syndrome (Barbosa et al, 2007, FASEB J 21: 3629; Chiang et al, 2015, PLoS ONE 10: e0134927).

Therefore, a fourth aspect of the present invention provides a compound, salt, solvate or prodrug according to the first or second aspect of the present invention, for use in therapy, in particular for use in treating or preventing a disease, disorder or condition associated with CD38 activity.

The fourth aspect of the present invention also provides a compound, salt, solvate or prodrug according to the first or second aspect of the present invention, for use in treating or preventing a CNS disease, a disease requiring treatment via the CNS, a neurodegenerative condition, a neurological disease, an age-related disorder, or an inflammatory disorder.

The fourth aspect of the present invention also provides a compound, salt, solvate or prodrug according to the first or second aspect of the present invention, for use in treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome.

A fifth aspect of the present invention provides a use of a compound, salt, solvate or prodrug according to the first or second aspect of the present invention, for the manufacture of a medicament for treating or preventing a disease, disorder or condition associated with CD38 activity.

The fifth aspect of the present invention also provides a use of a compound, salt, solvate or prodrug according to the first or second aspect of the present invention, for the manufacture of a medicament for treating or preventing a CNS disease, a disease requiring treatment via the CNS, a neurodegenerative condition, a neurological disease, an age-related disorder, or an inflammatory disorder.

The fifth aspect of the present invention also provides a use of a compound, salt, solvate or prodrug according to the first or second aspect of the present invention, for the manufacture of a medicament for treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome.

A sixth aspect of the present invention provides a method of treating or preventing a disease, disorder or condition associated with CD38 activity; the method comprising administering a therapeutically or prophylactically effective amount of a compound, salt, solvate or prodrug according to the first or second aspect of the present invention, to a patient in need thereof.

The sixth aspect of the present invention also provides a method of treating or preventing a CNS disease, a disease requiring treatment via the CNS, a neurodegenerative condition, a neurological disease, an age-related disorder, or an inflammatory disorder; the method comprising administering a therapeutically or prophylactically effective amount of a compound, salt, solvate or prodrug according to the first or second aspect of the present invention, to a patient in need thereof.

The sixth aspect of the present invention also provides a method of treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome; the method comprising administering a therapeutically or prophylactically effective amount of a compound, salt, solvate or prodrug according to the first or second aspect of the present invention, to a patient in need thereof.

Unless stated otherwise, in any of the fourth, fifth or sixth aspects of the invention, the subject or patient may be any human or other animal. Typically, the subject or patient is a mammal, more typically a human or a domesticated mammal such as a cow, pig, lamb, sheep, goat, horse, cat, dog, rabbit, mouse etc. Most typically, the subject is a human.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

For the above-mentioned therapeutic uses the dosage administered will, of course, vary with the compound employed, the mode of administration, the treatment desired and the disorder indicated. For example, the daily dosage of a compound of the invention (that is, a compound of formula (I), (II) or (III), or a pharmaceutically acceptable salt, solvate or prodrug thereof) by oral or parenteral administration may be in the range from 0.01 micrograms per kilogram body weight (μg/kg) to 500 milligrams per kilogram body weight (mg/kg). The desired dosage may be presented at an appropriate interval such as once every other day, once a day, twice a day, three times a day or four times a day.

The compounds and pharmaceutically acceptable salts, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound, salt, solvate or prodrug according to the first or second aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound, salt, solvate or prodrug according to the first or second aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, dipotassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally, ocularly, topically or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intradermal, intravenous, intramuscular, intra-articular, intrasynovial, intrasternal, intrathecal, intralesional, intracranial, intratracheal, intraperitoneal, intraarticular, and epidural injection or infusion techniques. The term topical as used herein includes transdermal, mucosal, sublingual and topical ocular administration.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution, and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to capsules, tablets, caplets, troches, lozenges, powders, granules, and aqueous suspensions, solutions, and dispersions. These dosage forms are prepared according to techniques well-known in the art of pharmaceutical formulation. In the case of tablets for oral use, carriers which are commonly used include lactose, sodium and calcium carbonate, sodium and calcium phosphate, and corn starch. Lubricating agents, such as magnesium stearate, stearic acid or tale, are also typically added. If desired, the tablets may be coated with a material, such as glyceryl monostearate or glyceryl distearate, to delay absorption in the gastrointestinal tract. Tablets may also be effervescent and/or dissolving tablets. For oral administration in capsule form, useful diluents include lactose and dried corn starch. When aqueous suspensions are administered orally, the active ingredient may be combined with emulsifying and suspending agents. If desired, certain sweetening and/or flavouring and/or colouring agents and/or preservatives may be added to any oral dosage form.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels, and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Depending on the mode of administration, the pharmaceutical composition will preferably comprise from 0.05 to 99% by weight, more preferably from 0.05 to 80% by weight, still more preferably from 0.1 to 70% by weight, and even more preferably from 0.1 to 50% by weight of active ingredient, all percentages by weight being based on total composition.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations, i.e. in the form of a kit.

Typically, the mode of administration selected is that most appropriate to the disorder, disease or condition to be treated or prevented. Where one or more further active agents are administered, the mode of administration may be the same as or different to the mode of administration of the compound or pharmaceutical composition of the invention.

Such combination products employ the compounds of this invention within the dosage range described herein and the other pharmaceutically active agent(s) within approved dosage ranges.

### Definitions

An “alkyl” group may be linear (i.e. straight-chained) or branched. Examples of alkyl groups include methyl, ethyl, n-propyl, iso-propyl, n-butyl, iso-butyl, sec-butyl, tert-butyl, n-pentyl, 2-methyl-1-butyl, 3-methyl-1-butyl, 3-methyl-2-butyl, and 2,2-dimethyl-1-propyl groups. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” group is an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” group is an unsaturated alkyl group having one or more carbon-carbon triple bonds. Examples of alkynyl groups include ethynyl, propargyl, but-1-ynyl and but-2-ynyl groups. Typically an alkynyl group is a C₂-C₁₂ alkynyl group. More typically an alkynyl group is a C₂-C₆ alkynyl group. An “alkynylene” group is similarly defined as a divalent alkynyl group.

A “cycloalkyl” group is a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may be monocyclic, bicyclic (e.g. bridged, fused or spiro), or polycyclic.

A “cycloalkenyl” group is a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may be monocyclic, bicyclic (e.g. bridged, fused or spiro), or polycyclic.

An “aryl” group is an aromatic hydrocarbyl ring. The term “aryl” includes monocyclic aromatic hydrocarbons (such as phenyl) and polycyclic fused-ring aromatic hydrocarbons (such as naphthyl, anthracenyl and phenanthrenyl). Unless stated otherwise, the term “aryl” does not include “heteroaryl”.

A “heterocyclic” group is a non-aromatic cyclic group which includes one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 14-membered heterocyclic group, which means it contains from 4 to 14 ring atoms. More typically, a heterocyclic group is a 4- to 10-membered heterocyclic group, which means it contains from 4 to 10 ring atoms. Heterocyclic groups include unsaturated heterocyclic groups (such as azetinyl, tetrahydropyridinyl, and 2-oxo-1H-pyridinyl) and saturated heterocyclic groups. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl and hexahydro-1H-pyrrolizinyl groups.

A “heteroaryl” group is an aromatic cyclic group which includes one or more carbon atoms and one or more (such as one, two, three or four) heteroatoms, e.g. N, O or S, in the ring structure. Typically, a heteroaryl group is a 5- to 14-membered heteroaryl group, which means it contains from 5 to 14 ring atoms. More typically, a heteroaryl group is a 5- to 10-membered heteroaryl group, which means it contains from 5 to 10 ring atoms. The term “heteroaryl” includes monocyclic aromatic heterocycles (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and tetrazinyl) and polycyclic fused-ring aromatic heterocycles (such as indolyl, benzofuranyl, benzothiophenyl, benzoxazolyl, benzisoxazolyl, benzothiazolyl, benzoisothiazolyl, benzimidazolyl, 1H-imidazo[4,5-b]pyridinyl, 1H-imidazo[4,5-c]pyridinyl, quinolinyl, isoquinolinyl, quinoxalinyl, quinazolinyl, phthalazinyl and cinnolinyl). Examples of heteroaryl groups include the following:

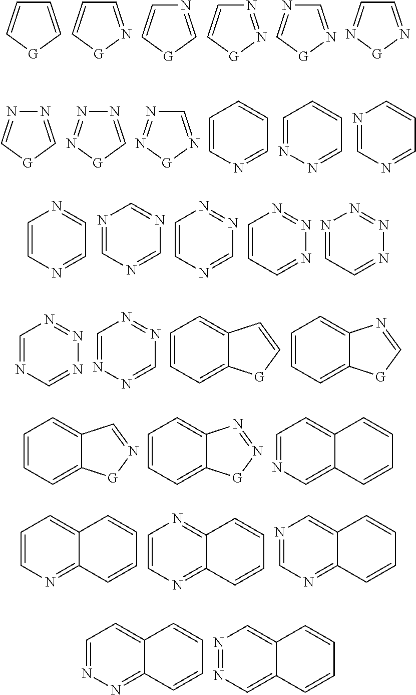

wherein G═O, S or NH.

Accordingly, a 5-membered heteroaryl group comprising two heteroatoms independently selected from N and S, may be a pyrazolyl, imidazolyl, thiazolyl or isothiazolyl group.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo. In one embodiment, halo is fluoro.

Unless stated otherwise, where a group is prefixed by the term “halo”, such as a “haloalkyl” or “halomethyl” group, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without the halo prefix. For example, a “halomethyl” group may contain one, two or three halo substituents. A “haloethyl” or “halophenyl” group may contain one, two, three, four or five halo substituents. Similarly, unless stated otherwise, where a group is prefixed by a specific halo group, it is to be understood that the group in question is substituted with one or more (such as one, two, three, four or five) of the specific halo groups. For example, the term “fluoromethyl” refers to a methyl group substituted with one, two or three fluoro groups, and the term “fluoroethyl” refers to an ethyl group substituted with one, two, three, four or five fluoro groups.

Similarly, unless stated otherwise, where a group is said to be “halo-substituted”, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without halo substitution. For example, a “halo-substituted methyl” group may contain one, two or three halo substituents. A “halo-substituted ethyl” or “halo-substituted phenyl” group may contain one, two, three, four or five halo substituents. Similarly, unless stated otherwise, where a group is said to be substituted with a specific halo group, such as a “fluoro-substituted” group, it is to be understood that the group in question is substituted with one or more (such as one, two, three, four or five) of the specific halo groups. For example, the term “fluoro-substituted methyl” refers to a methyl group substituted with one, two or three fluoro groups, and the term “fluoro-substituted ethyl” refers to an ethyl group substituted with one, two, three, four or five fluoro groups.

A “hydroxyalkyl” group is an alkyl group substituted with one or more (such as one, two or three) hydroxyl (—OH) groups. Typically a hydroxyalkyl group has one or two hydroxyl substituents, more typically a hydroxyalkyl group has one hydroxyl substituent.

Unless stated otherwise, any reference to an element is to be considered a reference to all isotopes of that element. Thus, for example, unless stated otherwise, any reference to hydrogen is considered to encompass all isotopes of hydrogen including ¹H, ²H (D) and ³H (T). Therefore, for the avoidance of doubt, it is noted that, for example, the terms “alkyl” and “methyl” include, for example, trideuteriomethyl.

Unless stated otherwise, any reference to a compound or group is to be considered a reference to all tautomers of that compound or group.

When any chemical group or moiety is described as substituted, it will be appreciated that the number and nature of substituents will be selected so as to avoid sterically undesirable combinations.

### EXAMPLES

The present invention will now be further explained by reference to the following illustrative examples, in which the starting materials and reagents used are available from commercial suppliers or prepared via literature procedures or procedures similar to the ones described in this application.

### Abbreviations

DIPEA N,N-diisopropylethylamine
DMF dimethylformamide
EtOAc ethyl acetate
EtOH ethanol
h hour
HATU 1-[bis(dimethylamino)methylene]-1H-1,2,3-triazolo[4,5-b]pyridinium 3-oxid hexafluorophosphate
HPLC high-performance liquid chromatography
MeCN acetonitrile
MeOH methanol
min minute
NMP N-methyl-2-pyrrolidone
PCy₃·BF₄ tricyclohexylphosphine tetrafluoroborate
Pd(dppf)Cl₂ 1,1′-bis(diphenylphosphino)ferrocene]dichloropalladium(II)
RT room temperature
SFC super critical fluid chromatography
T₃P propylphosphonic anhydride
TEA triethylamine
TFA trifluoroacetic acid
THF tetrahydrofuran
UPLC ultra-performance liquid chromatography

### General Procedures

Nuclear magnetic resonance (NMR) spectra were recorded at 400 MHz as stated and at 298.2K or 294.1K unless otherwise stated; the chemical shifts (δ) are reported in parts per million. Spectra were recorded using a Bruker® 400 AVANCE instrument fitted with a 5 mm iprobe or smart probe with instrument controlled by Bruker TopSpin 4.0.9 or Bruker TopSpin 4.1.1 software.

Reactions were monitored using one or more of the following. Agilent 1290 infinity II UPLC coupled with 6130 quadrupole LCMS: mobile phase A: 0.037% TFA in H₂O; mobile phase B: 0.018% TFA in CH₃CN; column: Xtimate® C18 2.1×30 mm, 3 μm; column temperature: 50° C.; sample temperature: RT; detection (nm): 220 nm and 254 nm; flow rate: 1.0 mL/min; analysis time: 4.0 min.; measured mass range: 100 to 1500 m/z.

Purity was assessed using the following: UPLC with UV (photodiode array) detection over a wide range of wavelengths, normally 220-254 nm, using Shimadzu® Nexera X2 UPLC controlled by Lab Solution software equipped with Acquity UPLC BEH, HSS or HSS T₃ C18 columns (2.1 mm id×50 mm long) operated at 50° C. Unless stated otherwise, mobile phases typically consisted of CH₃CN mixed with H₂O containing either 0.037% TFA or 0.225% HCOOH. Mass spectra were recorded with a Shimadzu single quadrupole mass spectrometer using DUIS ionisation.

Compounds were purified using Biotage or ISCO® instrument using normal phase chromatography on silica or by preparative high performance liquid chromatography (HPLC).

Preparative HPLC was performed using Gilson GX-281 system using Phenomenex C18 75×30 mm, 3 μm; Xtimate C18 100×30 mm, 10 μm; Xtimate C18 150×40 mm, 10 μm; Xtimate C18 150×40 mm, 10 μm; Phenomenex C18 75×30 mm, 3 μm or Gemini NX C18 10×150 mm, 5 μm columns at RT. Mobile phases typically consisted of CH₃CN mixed with H₂O containing either 0.225% formic acid or 0.05% ammonia+10 nM NH₄HCO₃, unless otherwise stated.

Super Critical Fluid Chromatography (SFC) chiral analysis were performed on a Waters UPCC with PDA Detector, using a flow rate of 4 mL/min, temperature of RT to 35° C. and a pressure of 1500 psi. Mobile phases typically consisted of supercritical CO₂ and a polar solvent such as CH₃CN, MeOH, EtOH or isopropanol. Column type and eluent are detailed for individual examples. Columns: Chiralpak OD-3 50×4.6 mm, 3 μm; Chiralpak AD-3 50×4.6 mm, 3 μm; Chiral NS-3 100×4.6 mm, 3 μm; Chiral MD-3 100×4.6 mm, 3 μm; Chiralpak IG 50×4.6 mm, 3 μm; (S,S)-Whelk-o-1.8 50×4.6 mm, 1.8 μm; Chiralpak OJ-3 100×4.6 mm, 3 μm; Detection: 220 nm; sample diluent: CH₃CN, MeOH; injection: 9 μl; isocratic ratio: 5% to 40% of mobile phase. ‘Room temperature’, as used in the present specification, means a temperature in the range from about 18° C. to about 25° C.

### SYNTHESIS OF INTERMEDIATES

### Intermediate 1: (1r,4r)—N¹-(2,2,2-trifluoroethyl)cyclohexane-1,4-diamine

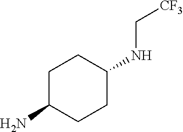 [SMILES: N[C@H]1CC[C@H](NCC(F)(F)F)CC1]

Step 1: A solution of (1r,4r)—N¹,N¹-dibenzylcyclohexane-1,4-diamine hydrochloride (6.30 g, 18.7 mmol), 2,2,2-trifluoroethyl trifluoromethanesulfonate (5.21 g, 22.5 mmol) and NEt₃ (9.47 g, 93.6 mmol) in CH₃CN (60 mL) was stirred at 70° C. for 12 h and extracted with EtOAc (80 mL×3). The combined organic layers were dried (Na₂SO₄), filtered, concentrated under reduced pressure and purified by flash silica gel chromatography (ISCO®; 40 g SepaFlash® Silica Flash Column, EtOAc/petroleum ether: 0 to 20%, 80 mL/min) to give (1r,4r)—N¹,N¹-dibenzyl-N⁴-(2,2,2-trifluoroethyl)cyclohexane-1,4-diamine (4.20 g, 10.9 mmol, 58.4%) as a white solid. ¹H NMR (400 MHz, DMSO-d₆): 7.16-7.38 (m, 10H), 3.56 (s, 4H), 3.12-3.23 (m, 2H), 2.28-2.43 (m, 2H), 2.02-2.13 (m, 1H), 1.90 (d, J=11.8 Hz, 2H), 1.80 (d, J=11.5 Hz, 2H), 1.29-1.45 (m, 2H), 0.77-0.91 (m, 2H). MS ES⁺: 377.4.

Step 2: A solution of (1r,4r)—N¹,N¹-dibenzyl-N⁴-(2,2,2-trifluoroethyl)cyclohexane-1,4-diamine (4.10 g, 10.9 mmol) in EtOH (40 mL) was treated with Pd(OH)₂ (956 mg, 1.36 mmol, 20% purity), degassed and purged with H₂ three times. The mixture was stirred at 25° C. for 12 h under H₂ atmosphere (40 psi) and filtered through Celite®. The filtrate was concentrated to afford the title compound (2.10 g, 89.1%) as a white solid. ¹H NMR (400 MHz, DMSO-d₆): 3.09-3.25 (m, 2H), 2.51 (s, 1H), 2.33 (s, 1H), 2.10 (d, J=4.6 Hz, 1H), 1.68-1.84 (m, 4H), 0.89-1.03 (m, 4H).

### Intermediate 2: (1r,4r)—N¹-(2,2-difluoroethyl)cyclohexane-1,4-diamine

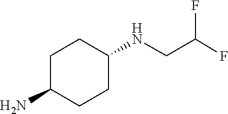 [SMILES: N[C@H]1CC[C@H](NCC(F)F)CC1]

Prepared as described for Intermediate 1 using (1r,4r)—N¹,N¹-dibenzylcyclohexane-1,4-diamine hydrochloride (prepared as described in US2011/0034470, 7.00 g, 20.9 mmol) and 2,2-difluoroethyl trifluoromethanesulfonate (5.36 g, 25.1 mmol) to give the title compound (1.30 g, 35%) as a white solid.

### Intermediate 3: (1r,4r)-N¹-methyl-N¹-(2,2,2-trifluoroethyl)cyclohexane-1,4-diamine

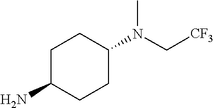

Step 1: A solution of (1r,4r)—N¹,N¹-dibenzyl-N⁴-(2,2,2-trifluoroethyl)cyclohexane-1,4-diamine (500 mg, 1.33 mmol) in THF (10 mL) was treated with NaH (159 mg, 3.98 mmol, 60% dispersion in oil) followed by dropwise addition of CH₃I (226 mg, 1.59 mmol). The mixture was stirred at 25° C. for 12 h and extracted with EtOAc (15 mL×3). The combined organic layers were washed with brine (20 mL), dried (Na₂SO₄), filtered, concentrated under reduced pressure and purified by flash silica gel chromatography (ISCO®; 12 g SepaFlash® Silica Flash Column, EtOAc/petroleum ether: 0 to 50%, 35 mL/min) to give (1r,4r)—N¹,N¹-dibenzyl-N⁴-methyl-N⁴-(2,2,2-trifluoroethyl)-cyclohexane-1,4-diamine (250 mg, 48%) as an off-white solid.

Step 2: A solution of (1r,4r)—N¹,N¹-dibenzyl-N⁴-methyl-N⁴-(2,2,2-trifluoroethyl)-cyclohexane-1,4-diamine (250 mg, 0.640 mmol) in MeOH (10 mL) was treated with 10% palladium on carbon (wetted with ca. 55% H₂O) (100 mg), and degassed and purged with H₂ three times. The mixture was stirred at 50° C. for 12 h under H₂ atmosphere (40 psi) and filtered through Celite®. The filtrate was concentrated to give the title compound (70 mg, 52%) as an off-white solid which was used for the next step without any further purification.

### Intermediate 4: 4-(dibenzylamino)cyclohexanone

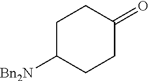

Prepared as described in WO2018/114700 using 1,4-dioxaspiro[4.5]decan-8-one (5.00 g, 32.0 mmol) and dibenzylamine (6.32 g, 32.0 mmol) to give the title compound (2.75 g, 21%) as a yellow oil. MS ES⁺: 294.1.

### Intermediate 5: 4-cyclopropyl-6-(1H-imidazol-1-yl)picolinic Acid

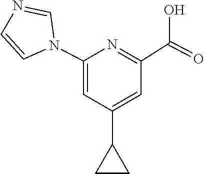 [SMILES: O=C(O)c1cc(C2CC2)cc(-n2ccnc2)n1]

Step 1: A solution of imidazole (1.24 g, 18.3 mmol) in THF (50 mL) was treated with NaH (876 mg, 21.91 mmol, 60% dispersion in oil) at 0° C., stirred for 0.5 h, treated with 2,6-dichloro-4-iodo-pyridine (5.00 g, 18.3 mmol), stirred at 60° C. for 12 h and cooled to RT. The mixture was quenched with sat. NH₄Cl (aq., 50 mL) at 0° C. and extracted with EtOAc (50 mL×3). The combined organic layers were washed with brine (100 mL), dried (Na₂SO₄), filtered, concentrated under reduced pressure and purified by flash silica gel chromatography (ISCO®; 80 g SepaFlash® Silica Flash Column, EtOAc/petroleum ether: 0 to 50%, 100 mL/min) to give 2-chloro-6-(1H-imidazol-1-yl)-4-iodopyridine (1.45 g, 26%) as a yellow solid. ¹H NMR (400 MHz, DMSO-d₆): 8.53 (s, 1H), 8.33 (d, J=0.8 Hz, 1H), 7.96 (d, J=0.8 Hz, 1H), 7.95 (t, J=1.4 Hz, 1H), 7.13 (s, 1H). MS ES⁺: 305.9.

Step 2: A mixture of 2-chloro-6-(1H-imidazol-1-yl)-4-iodopyridine (1.15 g, 3.76 mmol), cyclopropylboronic acid (647 mg, 7-53 mmol), K₃PO₄ (4.79 g, 22.59 mmol), and PCy₃·BF₄ (277 mg, 0.753 mmol) in H₂O (5 mL) and toluene (20 mL) was treated with Pd(OAc)₂ (169.02 mg, 0.753 mmol), degassed and purged with N₂ three times, stirred at 110° C. for 6 h under N₂ atmosphere, cooled to RT and filtered. The filtrate was concentrated under reduced pressure and purified by flash silica gel chromatography (ISCO®; 20 g SepaFlash® Silica Flash Column, EtOAc/petroleum ether: 0 to 35%, 80 mL/min) to give 2-chloro-4-cyclopropyl-6-(1H-imidazol-1-yl)pyridine (750 mg, 3.41 mmol, 90.7%) as a yellow solid. MS ES⁺: 220.0.

Step 3: A mixture of 2-chloro-4-cyclopropyl-6-(1H-imidazol-1-yl)pyridine (750 mg, 3.41 mmol) and NEt₃ (1.04 g, 10.2 mmol) in MeOH (10 mL) was treated with Pd(dppf)Cl₂ (250 mg, 0.341 mmol), degassed and purged with carbon monoxide three times, stirred at 80° C. for 16 h under a carbon monoxide atmosphere (50 psi), cooled to RT and filtered. The filtrate was concentrated under reduced pressure and purified by flash silica gel chromatography (ISCO®; 20 g SepaFlash® Silica Flash Column, EtOAc/petroleum ether: 0 to 60%, 80 mL/min) to give methyl 4-cyclopropyl-6-(1H-imidazol-1-yl)picolinate (650 mg, 2.67 mmol, 78.3%) as a brown solid. ¹H NMR (400 MHz, CDCl₃): 8.50 (s, 1H), 7.72 (s, 1H), 7.66 (d, J=1.2 Hz, 1H), 7.29 (d, J=1.2 Hz, 1H), 7.23 (s, 1H), 4.00 (s, 3H), 2.09-2.01 (m, 1H), 1.28-1.22 (m, 2H), 1.00-0.94 (m, 2H). MS ES⁺: 244.2.

Step 4: A solution of methyl 4-cyclopropyl-6-(1H-imidazol-1-yl)picolinate (650 mg, 2.67 mmol) and LiOH·H₂O (336 mg, 8.02 mmol) in THF (12 mL) and H₂O (4 mL) was stirred at 25° C. for 1 h, acidified using 1 M aq. HCl to pH=3-4 and concentrated under reduced pressure to give the title compound (1.00 g, crude) as a grey solid, which was used for the next step without further purification. MS ES⁺:230.2.

### Intermediate 6: (1r,4r)-4-(3,3-difluoropyrrolidin-1-yl)-cyclohexan-1-amine and Intermediate 7: (1s,4s)-4-(3,3-difluoropyrrolidin-1-yl)-cyclohexan-1-amine

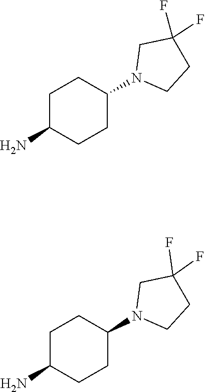 [SMILES: N[C@H]1CC[C@H](N2CCC(F)(F)C2)CC1 ; N[C@H]1CC[C@@H](N2CCC(F)(F)C2)CC1]

Step 1: A solution of 4-(dibenzylamino)cyclohexanone (Intermediate 4) (0.50 g, 1.70 mmol), 3,3-difluoropyrrolidine hydrochloride (269 mg, 1.87 mmol) and acetic acid (102 mg, 1.70 mmol) in CH₂Cl₂ (25 mL) was stirred at 25° C. for 1 h, treated with NaBH₃CN (214 mg, 3.41 mmol) at 0° C., warmed to 25° C. and stirred for 3 h. The mixture was treated with 1 M NaOH (aq., 75 mL) and extracted with CH₂Cl₂ (75 mL×3). The combined organic layers were washed with brine (75 mL), dried (Na₂SO₄), filtered, concentrated and purified by prep-HPLC (column: Xtimate C18 150×40 mm 10 μm, mobile phase A: H₂O (0.05% NH₃H₂O+10 mM NH₄HCO₃), mobile phase B: CH₃CN, 55-95% B, flow rate: 60 mL/min). The product was partitioned between CH₃CN (20 mL) and H₂O (100 mL). The aqueous solution was lyophilized to dryness to give (1r,4r)—N,N-dibenzyl-4-(3,3-difluoropyrrolidin-1-yl)-cyclohexan-1-amine (480 mg, 36%) and (1s,4s)—N,N-dibenzyl-4-(3,3-difluoropyrrolidin-1-yl)-cyclohexan-1-amine (322 mg, 0.752 mmol, 22.1%), both as a white solid.

(1r,4r)—N,N-dibenzyl-4-(3,3-difluoropyrrolidin-1-yl)-cyclohexan-1-amine: ¹H NMR (400 MHz, DMSO-d₆): 7.42-7.14 (m, 10H), 3.56 (s, 4H), 2.88 (t, J=13.6 Hz, 2H), 2.69 (t, J=6.6 Hz, 2H), 2.37 (t, J=10.9 Hz, 1H), 2.23-2.10 (m, 2H), 2.10-2.02 (m, 1H), 1.90 (d, J=11.5 Hz, 2H), 1.82 (d, J=11.5 Hz, 2H), 1.38 (q, J=11.5 Hz, 2H), 1.04-0.89 (m, 2H). MS ES⁺: 385.1.

(1s,4s)—N,N-dibenzyl-4-(3,3-difluoropyrrolidin-1-yl)-cyclohexan-1-amine: ¹H NMR (400 MHz, DMSO-d₆): 7.53-7.10 (m, 10H), 3.57 (s, 4H), 2.85 (t, J=13.8 Hz, 2H), 2.65 (t, J=6.8 Hz, 2H), 2.46-2.40 (m, 1H), 2.31-2.19 (m, 2H), 2.16 (s, 1H), 1.82 (d, J=13.3 Hz, 2H), 1.75-1.60 (m, 2H), 1.52 (d, J=10.1 Hz, 2H), 1.26-1.14 (m, 2H).

Step 2a: A solution of (1r,4r)—N,N-dibenzyl-4-(3,3-difluoropyrrolidin-1-yl)-cyclohexan-1-amine (300 mg, 0.780 mmol) in MeOH (10 mL) was treated with Pd(OH)₂ (300 mg, 0.427 mmol, 20% purity), degassed and purged with H₂ three times. The mixture was stirred at 50° C. for 12 h under H₂ atmosphere at 40 psi, filtered through Celite®, and concentrated under reduced pressure to give (1r,4r)-4-(3,3-difluoropyrrolidin-1-yl)-cyclohexan-1-amine (Intermediate 6) (158 mg, 0.619 mmol, 99.1% yield, 80% purity) as a yellow solid which was used for the next step without further purification. ¹H NMR (400 MHz, DMSO-d₆): 2.93-2.83 (m, 2H), 2.69 (t, J=7.0 Hz, 2H), 2.48-2.40 (m, 1H), 2.25-2.11 (m, 2H), 2.04-1.94 (m, 1H), 1.86-1.68 (m, 3H), 1.66-1.26 (m, 1H), 1.26-0.80 (m, 4H).

Step 2b: A solution of (1s,4s)—N,N-dibenzyl-4-(3,3-difluoropyrrolidin-1-yl)-cyclohexan-1-amine (320 mg, 0.832 mmol) in MeOH (10 mL) was treated with Pd(OH)₂ (200 mg, 0.285 mmol, 20% purity), degassed and purged with H₂ three times. The mixture was stirred at 50° C. for 12 h under H₂ atmosphere at 40 psi, filtered through Celite®, and concentrated under reduced pressure to give (1s,4s)-4-(3,3-difluoropyrrolidin-1-yl)-cyclohexan-1-amine (Intermediate 7) (168 mg, 0.822 mmol, 98.8%) as a white gum. ¹H NMR (400 MHz, DMSO-d₆): 3.16 (s, 1H), 2.96-2.81 (m, 2H), 2.73-2.62 (m, 3H), 2.29-2.15 (m, 2H), 2.14-2.06 (m, 1H), 1.73-1.56 (m, 2H), 1.50-1.32 (m, 5H).

### Intermediate 8: (1r,4r)—N¹-ethylcyclohexane-1,4-diamine

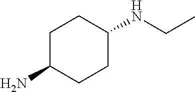

Step 1: To a solution of tert-butyl ((1r,4r)-4-aminocyclohexyl)carbamate (20 g, 93.3 mmol) in N,N-dimethylformamide (250 mL) was added K₂CO₃ (19.6 g, 140 mmol) and bromomethylbenzene (35.1 g, 205 mmol). At the same time, the internal temperature was maintained in the range of 25-30° C. The mixture was stirred at 25° C. for 12 hours. The reaction mixture was poured into water (600 mL) slowly while keeping the temperature in the range of 25-35° C. A white precipitation was formed and filtered. The filter cake was suspended into water (400 mL) and stirred for 20 min, the suspension was filtered and this sequence was repeated three times. The suspension was then filtered and the filter cake washed with water (150 mL) and n-hexane (150 mL). The filter cake was collected and dried over under reduced pressure to afford tert-butyl ((1r,4r)-4-(dibenzylamino)cyclohexyl)carbamate (30 g, 74.1 mmol, 79.4% yield, 97.5% chemical purity) as a yellow solid. 1H NMR (400 MHz, DMSO-d₆) 7.09-7.41 (m, 10H) 6.58 (br d, J=8.00 Hz, 1H) 3.56 (s, 4H) 3.16 (br d, J=8.13 Hz, 1H) 2.23-2.40 (m, 1H) 1.78 (br d, J=9.76 Hz, 4H) 1.38-1.49 (m, 2H) 1.35 (s, 9H) 0.92-1.06 (m, 2H).

Step 2: To a solution of tert-butyl ((1r,4r)-4-(dibenzylamino)cyclohexyl)carbamate (19 g, 48.16 mmol) in dichloromethane (50 mL) was added 4 M HCl in dioxane (4 M, 240.78 mL). The mixture was stirred at 25° C. for 0.5 hour. The reaction mixture was concentrated under reduced pressure to give a residue. The crude product (1r,4r)—N¹,N¹-dibenzylcyclohexane-1,4-diamine hydrochloride (21 g, 47.6 mmol, 98.8% yield, 75% chemical purity), a white solid, was used in next step without further purification. MS ES⁺: 295.2.

Step 3: To a solution of (1r,4r)—N¹,N¹-dibenzylcyclohexane-1,4-diamine hydrochloride (2 g, 6.79 mmol) and triethylamine (2.06 g, 20.4 mmol) in acetonitrile (20 mL) was added ethyl trifluoromethanesulfonate (1.09 g, 6.11 mmol). The mixture was stirred at 75° C. for 12 hours. The reaction mixture was diluted with H₂O (20 mL) and then extracted with EtOAc (3×30 mL). The combined organic layers were washed brine (30 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of Ethyl acetate:Methanol=10:1 @45 mL/min) to give (1r,4r)—N¹,N¹-dibenzyl-N⁴-ethylcyclohexane-1,4-diamine (2.1 g, crude) as a yellow solid. ¹H NMR (400 MHz, DMSO-d₆) 7.2-7.4 (m, 10H), 3.58 (d, 4H, J=4.9 Hz), 3.1-3.3 (m, 1H), 3.06 (br s, 1H), 2.93 (q, 1H, J=7.3 Hz), 2.3-2.5 (m, 1H), 1.8-2.1 (m, 4H), 1.2-1.6 (m, 4H), 1.1-1.2 (m, 3H).

Step 4: To a solution of (1r,4r)—N¹,N¹-dibenzyl-N⁴-ethylcyclohexane-1,4-diamine (2.1 g, 6.51 mmol) in MeOH (23 mL) was added Pd(OH)₂ (571.58 mg, 0.814 mmol, 20% purity). The reaction mixture was degassed and purged with H₂ (13.15 mg, 6.51 mmol) three times. The mixture was stirred at 50° C. for 12 hours under H₂ atmosphere (50 Psi). The reaction mixture was filtered through a celite pad and the filtrate was concentrated to afford the title compound (1.2 g, crude) as a white solid, which was used in the next step without further purification. ¹H NMR (400 MHz, DMSO-d₆) 4.91 (br s, 2H), 2.8-2.9 (m, 1H), 2.66 (q, 1H, J=7.1 Hz), 2.45 (q, 2H, J=7.2 Hz), 1.7-1.9 (m, 4H), 1.1-1.3 (m, 4H), 0.93 (t, 3H, J=7.1 Hz).

### Intermediate 9: N¹-(1,1,1-trifluoro-2-methylpropan-2-yl)cyclohexane-1,4-diamine hydrochloride

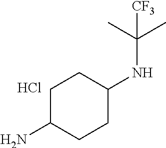

Step 1: To a solution of tert-butyl (4-oxocyclohexyl)carbamate (500 mg, 2.34 mmol) and 1,1,1-trifluoro-2-methylpropan-2-amine hydrochloride (460.17 mg, 2.81 mmol) in dichloromethane (5 mL) was added titanium(IV) propan-2-olate (1.33 g, 4.69 mmol) and after stirring for 1 hour, sodium cyanotrihydroborate (442 mg, 7.03 mmol) was added. The mixture was stirred at 25° C. for 12 hours. The reaction mixture was diluted with H₂O (50 mL) and extracted with EtOAc (3×30 mL). The combined organic layers were washed with brine (30 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜100% Ethyl acetate/Petroleum ether gradient @50 mL/min) to give tert-butyl (4-((1,1,1-trifluoro-2-methylpropan-2-yl)amino)cyclohexyl)carbamate (168 mg, 0.518 mmol, 22.1% yield) as a colourless solid. ¹H NMR (400 MHz, CDCl₃) 4.35 (br d, J=7.0 Hz, 1H), 3.43-3.26 (m, 1H), 2.77-2.60 (m, 1H), 2.00-1.92 (m, 2H), 1.86 (br d, J=12.2 Hz, 2H), 1.69-1.58 (m, 4H), 1.52 (s, 1H), 1.44 (s, 15H).

Step 2: To a solution of tert-butyl (4-((1,1,1-trifluoro-2-methylpropan-2-yl)amino)cyclohexyl)carbamate (160 mg, 0.493 mmol) in dichloromethane (2 mL) was added 4 M HCl in dioxane (4 M, 1.97 mL). The mixture was stirred at 25° C. for 1 hour. The reaction mixture was concentrated under reduced pressure to give the title compound (120 mg, 0.460 mmol, 93.3% yield) a white solid which was used without further purification. ¹H NMR (400 MHz, DMSO-d₆) 2.52 (br s, 1H), 2.13 (br d, J=12.2 Hz, 2H), 2.02 (br d, J=12.4 Hz, 2H), 1.94-1.85 (m, 2H), 1.81-1.71 (m, 3H), 1.62 (br s, 6H), 1.42 (br s, 1H).

### Intermediate 10: (1r,4S)—N¹—((S)-1,1,1-trifluoropropan-2-yl)cyclohexane-1,4-diamine

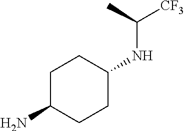

Step 1: To a solution of (S)-1,1,1-trifluoropropan-2-amine hydrochloride (500 mg, 3.34 mmol) in dichloromethane (10 mL) was added triethylamine (846 mg, 8.36 mmol) and the reaction mixture stirred for 0.5 hour before 4-(dibenzylamino)cyclohexan-1-one (818 mg, 2.79 mmol) and titanium(IV) propan-2-olate (1.58 g, 5.57 mmol) were added. The reaction was stirred for 1 hour before sodium cyanotrihydroborate (525 mg, 8.36 mmol) was added. The mixture was stirred at 25° C. for 13.5 hours. The mixture was quenched by EtOH (30 mL) then concentrated to the crude product. The crude product was purified by FCC (ISCO®; 4 g SepaFlash® Silica Flash Column, Eluent of 0˜16% Ethyl acetate/Petroleum ether gradient @60 mL/min) to give (1S,4r)—N¹,N¹-dibenzyl-N⁴-((S)-1,1,1-trifluoropropan-2-yl)cyclohexane-1,4-diamine (300 mg, 0.768 mmol, 27.6% yield) as a white solid. ¹H NMR (400 MHz, DMSO-d₆): 7.37-7.25 (m, 8H), 7.22-7.15 (m, 2H), 3.55 (s, 4H), 3.31-3.19 (m, 1H), 2.45-2.31 (m, 2H), 1.93 (br d, J=12.4 Hz, 1H), 1.87-1.73 (m, 3H), 1.63 (br s, 1H), 1.44-1.29 (m, 2H), 1.10 (d, J=6.8 Hz, 3H), 0.92-0.76 (m, 2H).

Step 2: To a solution of (1S,4r)—N¹,N¹-dibenzyl-N⁴-((S)-1,1,1-trifluoropropan-2-yl)cyclohexane-1,4-diamine (300 mg, 0.768 mmol) in EtOH (4 mL) was added Pd(OH)₂ (107.89 mg, 0.154 mmol, 20% purity) under N₂ atmosphere. The suspension was degassed and purged with H₂ 3 times. The mixture was stirred under H₂ atmosphere (40 psi) at 25° C. for 12 hours. The mixture was filtered and the filtrate was concentrated under reduced pressure to yield the title compound (115 mg, 0.547 mmol, 71.2% yield) as a white solid, which was used without further purification. ¹H NMR (400 MHz, DMSO-d₆): 2.40 (br s, 3H), 1.95-1.58 (m, 5H), 1.56-1.26 (m, 1H), 1.17-1.08 (m, 3H), 1.07-0.76 (m, 4H).

### Intermediate 11: (1r,4R)—N¹—((R)-1,1,1-trifluoropropan-2-yl)cyclohexane-1,4-diamine

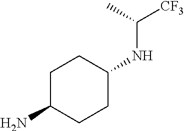

Step 1: To a solution of (R)-1,1,1-trifluoropropan-2-amine hydrochloride (500 mg, 3.34 mmol) in dichloromethane (10 mL) was added triethylamine (846 mg, 8.36 mmol) and the reaction mixture was stirred for 0.5 hour before 4-(dibenzylamino)cyclohexan-1-one (818 mg, 2.79 mmol) and titanium(IV) propan-2-olate (1.58 g, 5.57 mmol) were added. The reaction was stirred for 1 hour before sodium cyanotrihydroborate (525 mg, 8.36 mmol) was added. The mixture was stirred at 25° C. for 12 hours. The reaction mixture was concentrated under reduced pressure to give a residue. The residue was purified by column chromatography (SiO₂, Petroleum ether/Ethyl acetate=i/o to 5/1) to give (1R,4r)—N¹,N¹-dibenzyl-N⁴-((R)-1,1,1-trifluoropropan-2-yl)cyclohexane-1,4-diamine (425 mg, 1.09 mmol, 39.1% yield) as a colourless oil. ¹H NMR (400 MHz, CDCl₃) 7.27 (s, 10H), 3.62 (s, 4H), 3.21 (td, J=7.0, 14.0 Hz, 1H), 2.63-2.44 (m, 2H), 1.98-1.87 (m, 4H), 1.44-1.37 (m, 2H), 1.21 (d, J=6.9 Hz, 3H), 1.05-0.93 (m, 2H), 0.79 (br s, 1H).

Step 2: To a solution of (1R,4r)—N¹,N¹-dibenzyl-N⁴-((R)-1,1,1-trifluoropropan-2-yl)cyclohexane-1,4-diamine (425.00 mg, 1.09 mmol) in EtOH (10 mL) was added Pd(OH)₂ (200 mg, 0.285 mmol, 20% purity). The reaction mixture was degassed and purged with H₂ three times. The mixture was stirred at 25° C. for 12 hours under H₂ (40 psi) atmosphere. The reaction mixture was concentrated under reduced pressure to give the title compound (45 mg, 0.214 mmol, 19.7% yield) as a colourless oil, which was used without further purification. ¹H NMR (400 MHz, DMSO-d₆) 3.26 (br s, 1H), 2.48-2.35 (m, 2H), 2.14-1.98 (m, 1H), 1.87-1.61 (m, 6H), 1.42 (br dd, J=10.4, 13.3 Hz, 1H), 1.17-1.10 (m, 3H).

### Intermediate 12: 2,2,2-trifluoroethyl-1,1-d₂-4-methylbenzenesulfonate

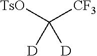 [SMILES: [2H]C([2H])(OS(=O)(=O)c1ccc(C)cc1)C(F)(F)F]

To a solution of 2,2,2-trifluoroethan-1,1-d₂-1-ol-d (100 mg, 0.970 mmol) in dichloromethane (1 mL) was added triethylamine (353 mg, 3.49 mmol). Then 4-methylbenzenesulfonyl chloride (231 mg, 1.21 mmol) was added at 0° C. The mixture was stirred at 20° C. for 2 hours. The reaction mixture was concentrated under reduced pressure. The residue was purified by prep. TLC (Petroleum ether:Ethyl acetate=5:1, Rf=0.48) to give the title compound (190 mg, 0.742 mmol, 76.4% yield) as a light-yellow liquid. ¹H NMR (400 MHz, CDCl₃) 7.88-7.78 (m, 2H), 7.44-7.34 (m, 2H), 2.51-2.45 (m, 3H).

### SYNTHESIS OF EXAMPLES

### Example 1: 2-(1H-imidazol-1-yl)-6-methyl-N-((1r,4r)-4-((2,2,2-trifluoroethyl)amino)cyclohexyl)pyrimidine-4-carboxamide

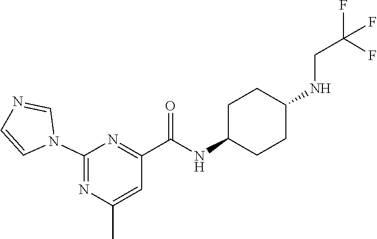 [SMILES: Cc1cc(C(=O)N[C@H]2CC[C@H](NCC(F)(F)F)CC2)nc(-n2ccnc2)n1]

Step 1: A solution of methyl 2-chloro-6-methyl-pyrimidine-4-carboxylate (2.00 g, 10.7 mmol), imidazole (875 mg, 12.8 mmol) and DIPEA (4.16 g, 32 mmol) in DMF (20 mL) was stirred at 100° C. for 4 h and concentrated under reduced pressure. Purification of the residue by flash silica gel chromatography (ISCO®, 40 g SepaFlash® Silica Flash Column, petroleum ether/EtOAc 0 to 60%, 100 mL/min) gave methyl 2-(1H-imidazol-1-yl)-6-methyl-pyrimidine-4-carboxylate (1.35 g, 6.19 mmol, 58%) as a pale-yellow solid. MS ES⁺: 219.0.

Step 2: A solution of methyl 2-(1H-imidazol-1-yl)-6-methyl-pyrimidine-4-carboxylate (1.35 g, 6.19 mmol) in THF (15 mL) was treated with 2 M LiOH (aq., 6.19 mL), stirred at 25° C. for 2 h and concentrated under reduced pressure. The residue was neutralized by 1 M HCl (aq.) to pH=2-3 and concentrated under reduced pressure to give 2-(1H-imidazol-1-yl)-6-methyl-pyrimidine-4-carboxylic acid (2.75 g, crude, 50% purity) as an off-white solid which was used for the next step without further purification. MS ES⁺: 205.0.

Step 3: A solution of 2-(1H-imidazol-1-yl)-6-methyl-pyrimidine-4-carboxylic acid (49.9 mg, 0.122 mmol, 50% purity), (1r,4r)-N¹-(2,2,2-trifluoroethyl)cyclohexane-1,4-diamine (Intermediate 1) (20 mg, 0.102 mmol) and NEt₃ (30.9 mg, 0.306 mmol) in DMF (2 mL) was treated with HATU (46.5 mg, 0.122 mmol), stirred at 25° C. for 1 h, cooled to RT and extracted with EtOAc (3×10 mL). The combined organic layers were washed with brine (20 mL), dried (Na₂SO₄), filtered and concentrated under reduced pressure to give a residue, which was purified by prep-HPLC (column: Gemini NX C18 10×150 mm 5 μm, mobile phase A: H₂O (0.225% HCOOH), mobile phase B: CH₃CN, 0-30% B, flow rate: 25 mL/min). The purified product was partitioned between CH₃CN (2 mL) and H₂O (10 mL) and lyophilized to give the title compound (11.4 mg, 0.029 mmol, 28.8%) as an off-white solid. ¹H NMR (400 MHz, DMSO-d₆): 8.95 (d, J=1.3 Hz, 1H), 8.79 (d, J=8.6 Hz, 1H), 8.21 (s, 1H), 7.82 (s, 1H), 7.18 (s, 1H), 3.84-3.80 (m, 1H), 3.26 (d, J=10.3 Hz, 3H), 2.63 (s, 3H), 2.45 (d, J=4.0 Hz, 1H), 1.96 (d, J=12.0 Hz, 2H), 1.82 (d, J=10.0 Hz, 2H), 1.54 (d, J=13.8 Hz, 2H), 1.13 (d, J=12.9 Hz, 2H). MS ES⁺: 383.0. UPLC purity: 98.6%. SFC chiral purity: 99.2%.

### Example 2: N-((1r,4r)-4-((2,2-difluoroethyl)amino)cyclohexyl)-2-(1H-imidazol-1-yl)-6-methyl-pyrimidine-4-carboxamide

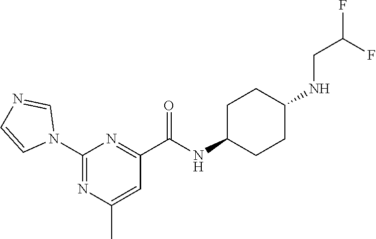 [SMILES: Cc1cc(C(=O)N[C@H]2CC[C@H](NCC(F)F)CC2)nc(-n2ccnc2)n1]

A solution of 2-(1H-imidazol-1-yl)-6-methyl-pyrimidine-4-carboxylic acid (55 mg, 0.135 mmol, 50% purity), (1r,4r)-N¹-(2,2-difluoroethyl)cyclohexane-1,4-diamine (Intermediate 2) (20 mg, 0.112 mmol) and NEt₃ (34 mg, 0.337 mmol) in DMF (3 mL) was treated with HATU (51.2 mg, 0.135 mmol), stirred at 25° C. for 1 h and extracted with EtOAc (3×10 mL). The combined organic layers were washed with brine (20 mL), dried (Na₂SO₄), filtered, concentrated, and purified by prep-HPLC (column: Gemini NX C18 10×150 mm 5 μm, mobile phase A: H₂O (0.225% HCOOH), mobile phase B: CH₃CN, 0-30% B, flow rate: 25 mL/min). The product was partitioned between CH₃CN (2 mL) and H₂O (10 mL) and lyophilized to give the title compound (15.9 mg, 0.044 mmol, 39%) as an off-white solid. ¹H NMR (400 MHz, DMSO-d₆): 8.95 (s, 1H), 8.80 (d, J=8.6 Hz, 1H), 8.20 (s, 1H), 7.82 (s, 1H), 7.17 (s, 1H), 6.10-5.82 (m, 1H), 3.87-3.76 (m, 1H), 2.93 (dt, J=4.3, 15.9 Hz, 2H), 2.63 (s, 3H), 2.49-2.38 (m, 2H), 1.96 (d, J=10.9 Hz, 2H), 1.82 (d, J=10.3 Hz, 2H), 1.60-1.48 (m, 2H), 1.18-1.07 (m, 2H). MS ES⁺: 365.4. UPLC purity: 99.4%. SFC chiral purity: 99-3%.

### Example 3: 6-cyclopropyl-N-((1r,4r)-4-((2,2-difluoroethyl)amino)cyclohexyl)-2-(1H-imidazol-1-yl)pyrimidine-4-carboxamide

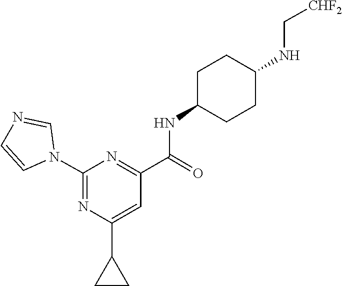 [SMILES: O=C(N[C@H]1CC[C@H](NCC(F)F)CC1)c1cc(C2CC2)nc(-n2ccnc2)n1]

Step 1: A solution of methyl 2-chloro-6-cyclopropyl-pyrimidine-4-carboxylate (3.60 g, 16.9 mmol) in DMF (20 mL) was treated with DIPEA (6.56 g, 50.8 mmol) and imidazole (3.46 g, 50.79 mmol), stirred at 100° C. for 12 h, cooled to RT and extracted with EtOAc (3×30 mL). The combined organic layers were washed with brine (50 mL), dried (Na₂SO₄) and filtered. The filtrate was evaporated and the residue purified by flash silica gel chromatography (ISCO®; 40 g SepaFlash® Silica Flash Column, EtOAc in petroleum ether: 0 to 100%, 35 mL/min) to give methyl 6-cyclopropyl-2-(1H-imidazol-1-yl)-pyrimidine-4-carboxylate (1.60 g, 6.55 mmol, 38%) as an off-white solid.

Step 2: A solution of methyl 6-cyclopropyl-2-(1H-imidazol-1-yl)-pyrimidine-4-carboxylate (1.60 g, 6.55 mmol) in THF (10 mL) was treated with LiOH (157 mg, 6.55 mmol) and H₂O (118 mg, 6.55 mmol) and stirred at 25° C. for 2 h. The mixture was adjusted to pH<7 using 1 M HCl (aq.). The resulting white precipitate was collected by filtration to give 6-cyclopropyl-2-(1H-imidazol-1-yl)-pyrimidine-4-carboxylic acid (1.40 g, 6.08 mmol, 93%) as an off-white solid which was used for the next step without further purification. MS ES⁺: 230.9.

Step 3: Prepared as described for Example 2 using 6-cyclopropyl-2-(1H-imidazol-1-yl)-pyrimidine-4-carboxylic acid (50 mg, 0.217 mmol) and (1r,4r)—N¹-(2,2-difluoroethyl)cyclohexane-1,4-diamine (Intermediate 2) (38.7 mg, 0.217 mmol) to give the title compound (30.5 mg, 0.076 mmol, 35%) as an off-white solid. ¹H NMR (400 MHz, DMSO-d₆): 8.90 (s, 1H), 8.76 (d, J=8.8 Hz, 1H), 8.16 (t, J=1.3 Hz, 1H), 7.86 (s, 1H), 7.14 (s, 1H), 6.09 (t, J=4.3 Hz, 0.25H), 5.95 (t, J=4.3 Hz, 0.5H), 5.81 (t, J=4.3 Hz, 0.25H), 3.87-3.76 (m, 1H), 3.34 (s, 1H), 2.92 (dt, J=4.3, 15.9 Hz, 2H), 2.43-2.34 (m, 2H), 1.95 (d, J=11.4 Hz, 2H), 1.82 (d, J=10.5 Hz, 2H), 1.54 (d, J=12.5 Hz, 2H), 1.24-1.18 (m, 4H), 1.12 (d, J=13.4 Hz, 2H). MS ES⁺: 391.2. UPLC purity: 98.0%. SFC chiral purity: 100%.

### Example 4: 6-methyl-2-(1-methyl-1H-imidazol-5-yl)-N-((1r,4r)-4-((2,2,2-trifluoroethyl)amino)cyclohexyl)pyrimidine-4-carboxamide

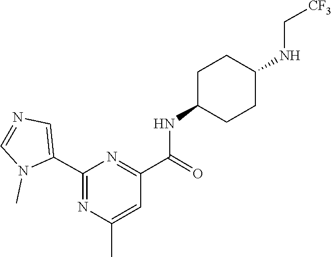 [SMILES: Cc1cc(C(=O)N[C@H]2CC[C@H](NCC(F)(F)F)CC2)nc(-c2cncn2C)n1]

Step 1: A mixture of 1-methyl-5-(4,4,5,5-tetramethyl-1,3,2-dioxaborolan-2-yl)imidazole (200 mg, 0.961 mmol), methyl 2-chloro-6-methyl-pyrimidine-4-carboxylate (149 mg, 0.801 mmol) and Cs₂CO₃ (522 mg, 1.60 mmol) in dioxane (8 mL) and H₂O (2 mL) was treated with Pd(dppf)Cl₂·CH₂Cl₂ (65.4 mg, 0.080 mmol), stirred at 90° C. for 2 h under N₂, cooled to RT and filtered. The filtrate was concentrated to dryness to give 6-methyl-2-(1-methyl-1H-imidazol-5-yl)-pyrimidine-4-carboxylic acid (175 mg, crude) as a black solid which was used for the next step without further purification.

Step 2: A mixture of 6-methyl-2-(1-methyl-1H-imidazol-5-yl)-pyrimidine-4-carboxylic acid (158 mg, 0.724 mmol) and (1r,4r)—N¹-(2,2,2-trifluoroethyl)cyclohexane-1,4-diamine (Intermediate 1) (170.49 mg, 0.869 mmol) in CH₂Cl₂ (6 mL) was treated with DIPEA (281 mg, 2.17 mmol) and T₃P (921 mg, 1.45 mmol, 50% of purity in EtOAc), stirred at 25° C. for 0.5 h and extracted with CH₂Cl₂ (3×30 mL). The combined organic layers were dried (Na₂SO₄) and filtered. The filtrate was evaporated and the residue purified by flash silica gel chromatography (ISCO®; 4 g SepaFlash® Silica Flash Column, petroleum ether/EtOAc: 0 to 100%, 45 mL/min) to give the title compound (59-7 mg, 0.150 mmol, 21%) as a white solid. ¹H NMR (400 MHz, DMSO-d₆): 8.44 (d, J=8.6 Hz, 1H), 8.00 (s, 1H), 7.85 (s, 1H), 7.66 (s, 1H), 4.05 (s, 3H), 3.88-3.71 (m, 1H), 3.29-3.21 (m, 2H), 2.58 (s, 3H), 2.47-2.36 (m, 1H), 2.28-2.18 (m, 1H), 1.99-1.88 (m, 2H), 1.88-1.80 (m, 2H), 1.57-1.43 (m, 2H), 1.19-1.06 (m, 2H). MS ES⁺: 397.0. UPLC purity: 99.3%. SFC chiral purity: 100%.

### Example 5: 6-methyl-2-(thiazol-5-yl)-N-((1r,4r)-4-((2,2,2-trifluoroethyl)amino)cyclohexyl)pyrimidine-4-carboxamide

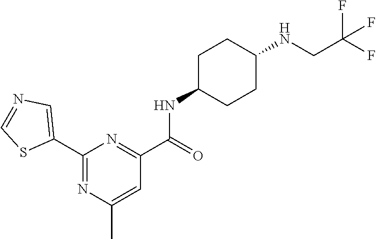 [SMILES: Cc1cc(C(=O)N[C@H]2CC[C@H](NCC(F)(F)F)CC2)nc(-c2cncs2)n1]

Step 1: A mixture of methyl 2-chloro-6-methyl-pyrimidine-4-carboxylate (300 mg, 1.61 mmol), 5-(4,4,5,5-tetramethyl-1,3,2-dioxaborolan-2-yl)thiazole (408 mg, 1.93 mmol) and Cs₂CO₃ (1.05 g, 3.22 mmol) in dioxane (12 mL) and H₂O (3 mL) was treated with Pd(dppf)Cl₂·CH₂Cl₂ (131 mg, 0.161 mmol), stirred at 90° C. for 1 h under N₂, cooled to RT and filtered. The filtrate was concentrated to dryness to give 6-methyl-2-(thiazol-5-yl)-pyrimidine-4-carboxylic acid (360 mg, crude) as a black solid which was used for the next step without further purification.

Step 2: A mixture of 6-methyl-2-(thiazol-5-yl)-pyrimidine-4-carboxylic acid (140 mg, 0.633 mmol) and (1r,4r)-N¹-(2,2,2-trifluoroethyl)cyclohexane-1,4-diamine (Intermediate 1) (149 mg, 0.759 mmol) in CH₂Cl₂ (2 mL) was treated with DIPEA (245 mg, 1.90 mmol) and T₃P (805 mg, 1.27 mmol, 50% of purity in EtOAc), stirred at 25° C. for 0.5 h and extracted with CH₂Cl₂ (30 mL×3). The combined organic layers were dried (Na₂SO₄) and filtered. The filtrate was evaporated and the residue purified by flash silica gel chromatography (ISCO®; 4 g SepaFlash® Silica Flash Column, EtOAc/petroleum ether: 0 to 100%, 45 mL/min) to give the title compound (39-5 mg, 14.9%) as a white solid. ¹H NMR (400 MHz, DMSO-d₆): 9.28 (s, 1H), 9.06 (s, 1H), 8.68 (d, J=8.6 Hz, 1H), 7.78 (s, 1H), 3.86-3.76 (m, 1H), 3.30-3.21 (m, 2H), 2.60 (s, 3H), 2.46-2.40 (m, 1H), 2.30-2.19 (m, 1H), 1.97-1.81 (m, 4H), 1.61-1.48 (m, 2H), 1.18-1.08 (m, 2H). MS ES⁺: 400.1. UPLC purity: 95.7%. SFC chiral purity: 100%.

### Example 6: 6-ethyl-2-(1H-imidazol-1-yl)-N-((1r,4r)-4-((2,2,2-trifluoroethyl)amino)cyclohexyl)pyrimidine-4-carboxamide

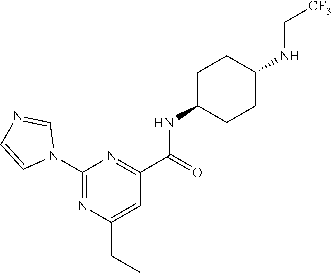 [SMILES: CCc1cc(C(=O)N[C@H]2CC[C@H](NCC(F)(F)F)CC2)nc(-n2ccnc2)n1]

Step 1: A mixture of methyl 2-chloro-6-vinyl-pyrimidine-4-carboxylate (1.02 g, 5.14 mmol) and PtO₂ (116.62 mg, 0.514 mmol) in EtOAc (20 mL) was degassed and purged with H₂ three times, and stirred at 25° C. for 1 h under H₂ atmosphere (15 psi). The mixture was diluted with EtOAc (20 mL) and filtered. The filtrate was concentrated under reduced pressure to give methyl 2-chloro-6-ethyl-pyrimidine-4-carboxylate (780 mg, 3.89 mmol, 76%) as a brown liquid, which was used for the next step without further purification.

Step 2: A mixture of methyl 2-chloro-6-ethyl-pyrimidine-4-carboxylate (780 mg, 3.89 mmol), imidazole (265 mg, 3.89 mmol) and DIPEA (1.51 g, 11.66 mmol) in DMF (10 mL) was stirred at 100° C. for 12 h under N₂, cooled to RT and concentrated under reduced pressure. The resulting residue was purified by flash silica gel chromatography (ISCO®; 20 g SepaFlash® Silica Flash Column, EtOAc/petroleum ether: 0 to 100%, 80 mL/min) to give methyl 6-ethyl-2-(1H-imidazol-1-yl)-pyrimidine-4-carboxylate (340 mg, 1.46 mmol, 38%) as a brown solid. ¹H NMR (400 MHz, DMSO-d₆): 8.58 (s, 1H), 7.94 (s, 1H), 7.87 (s, 1H), 7.16 (s, 1H), 3.95 (s, 3H), 2.93 (q, J=7.4 Hz, 2H), 1.30 (t, J=7.6 Hz, 3H).

Step 3: A solution of methyl 6-ethyl-2-(1H-imidazol-1-yl)-pyrimidine-4-carboxylate (340 mg, 1.46 mmol) in THF (3 mL) was treated with 1 M LiOH (aq., 4.39 mL) and stirred at 25° C. for 1 h. The pH of the mixture was adjusted pH<7 by adding 1 M HCl (aq). The mixture was concentrated under reduced pressure to give 6-ethyl-2-(1H-imidazol-1-yl)-pyrimidine-4-carboxylic acid (556 mg, crude) as a yellow solid, which was used for the next step without further purification.

Step 4: A solution of 6-ethyl-2-(1H-imidazol-1-yl)-pyrimidine-4-carboxylic acid (100 mg, 0.458 mmol) and (1r,4r)-N¹-(2,2,2-trifluoroethyl)cyclohexane-1,4-diamine (Intermediate 1) (89.92 mg, 0.458 mmol) in DMF (1 mL) was treated with DIPEA (177.68 mg, 1.37 mmol) followed by dropwise addition of T₃P (437.44 mg, 0.687 mmol, 50% purity in EtOAc). The resulting mixture was stirred at 25° C. for 1 h, diluted with sat. NaHCO₃ (aq., 10 mL) and extracted with EtOAc (3×15 mL). The combined organic layers were washed with 4% aq. LiCl, dried (Na₂SO₄), filtered and concentrated under reduced pressure. The resulting residue was purified by prep-HPLC (Column: XtimateC18 100×30 mm 10 μm, mobile phase A: H₂O (HCOOH), mobile phase B: CH₃CN, 0-30% B, flow rate: 25 mL/min). The product was partitioned between CH₃CN (2 mL) and H₂O (10 mL) and lyophilized to give the title product (40 mg, 0.099 mmol, 22%) as a white solid. ¹H NMR (400 MHz, DMSO-d₆): 8.95 (s, 1H), 8.79 (d, J=8.76 Hz, 1H), 8.21 (s, 1H), 7.81 (s, 1H), 7.16 (s, 1H), 3.81 (dd, J=7.88, 4.13 Hz, 1H), 3.26 (dd, J=10.19, 4.69 Hz, 2H), 2.91 (q, J=7.50 Hz, 2H), 2.41-2.45 (m, 1H), 2.16-2.29 (m, 1H), 1.95 (d, J=12.13 Hz, 2H), 1.81 (d, J=10.13 Hz, 2H), 1.47-1.61 (m, 2H), 1.29 (t, J=7.57 Hz, 3H), 1.07-1.18 (m, 2H). MS ES⁺: 397.4. UPLC purity: 97.4%. SFC chiral purity: 100%.

### Example 7: 2-(1H-imidazol-1-yl)-6-methyl-N-((1r,4r)-4-(methyl(2,2,2-trifluoroethyl)amino)cyclohexyl)pyrimidine-4-carboxamide

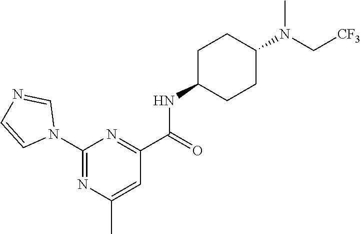 [SMILES: Cc1cc(C(=O)N[C@H]2CC[C@H](N(C)CC(F)(F)F)CC2)nc(-n2ccnc2)n1]

Step 1: A solution of 2-(1H-imidazol-1-yl)-6-methyl-pyrimidine-4-carboxylic acid (81.6 mg, 0.400 mmol), (1r,4r)-N¹-methyl-N¹-(2,2,2-trifluoroethyl)cyclohexane-1,4-diamine (Intermediate 3) (70 mg, 0.333 mmol) and NEt₃ (101 g, 0.999 mmol) in DMF (1 mL) was treated with HATU (189 mg, 0.499 mmol) and stirred at 25° C. for 1 h. The mixture was filtered and the filtrate concentrated to give a residue which was purified by prep-HPLC (Column: Phenomenex C18 75×30 mm 3 μm, mobile phase A: H₂O (NH₃·H₂O+NH₄HCO₃), mobile phase B: CH₃CN, 7-37% B, flow rate: 25 mL/min). The product was partitioned between CH₃CN (2 mL) and H₂O (10 mL). The aqueous solution was lyophilized to dryness to give the title compound (10.09 mg, 0.025 mmol, 7.4%) as a white solid. ¹H NMR (400 MHz, CD₃OD): 8.92 (s, 1H), 8.19 (s, 1H), 7.86 (s, 1H), 7.15 (s, 1H), 3.82-3.96 (m, 1H), 3.06-3.16 (m, 2H), 2.68 (s, 3H), 2.52-2.61 (m, 1H), 2.47 (s, 3H), 2.04 (br. d, J=11.26 Hz, 2H), 1.93 (br. d, J=11.76 Hz, 2H), 1.52-1.65 (m, 2H), 1.39-1.51 (m, 2H). MS ES⁺: 397.2. UPLC purity: 96.5%. SFC chiral purity: 100%.

### Example 8: N-((1r,4r)-4-((2,2-difluoroethyl)amino)cyclohexyl)-6-methyl-2-(thiazol-5-yl)pyrimidine-4-carboxamide

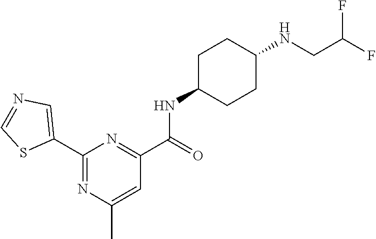 [SMILES: Cc1cc(C(=O)N[C@H]2CC[C@H](NCC(F)F)CC2)nc(-c2cncs2)n1]

Prepared as described for Example 5 using 6-methyl-2-(thiazol-5-yl)-pyrimidine-4-carboxylic acid (140 mg, 0.633 mmol) and (1r,4r)—N¹-(2,2-difluoroethyl)cyclohexane-1,4-diamine (Intermediate 2) (135 mg, 0.759 mmol) to give the title compound (13.0 mg, 0.033 mmol, 5.2%) as a white solid. ¹H NMR (400 MHz, DMSO-d₆): 9.27 (s, 1H), 9.05 (s, 1H), 8.71-8.62 (m, 1H), 7.78 (s, 1H), 6.12-5.79 (m, 1H), 3.87-3.74 (m, 1H), 3.01-2.85 (m, 2H), 2.60 (s, 3H), 2.47-2.38 (m, 1H), 1.99-1.80 (m, 5H), 1.62-1.47 (m, 2H), 1.20-1.05 (m, 2H). MS ES⁺: 382.1. UPLC purity: 96.7%. SFC chiral purity: 100%.

### Example 9: N-((1r,4r)-4-((2-fluoroethyl)amino)cyclohexyl)-2-(1H-imidazol-1-yl)-6-methyl-pyrimidine-4-carboxamide formate

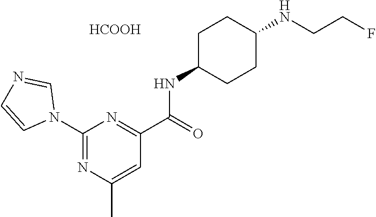 [SMILES: Cc1cc(C(=O)N[C@H]2CC[C@H](NCCF)CC2)nc(-n2ccnc2)n1 ; "HCOOH"]

Step 1: A solution of tert-butyl ((1r,4r)-4-(dibenzylamino)cyclohexyl)carbamate (19 g, 48.2 mmol) in CH₂Cl₂ (50 mL) was treated with 4 M HCl in dioxane (241 mL), stirred at 25° C. for 0.5 h and concentrated under reduced pressure to give (1r,4r)—N¹,N¹-dibenzylcyclohexane-1,4-diamine hydrochloride (21 g, 98.8% yield, 75% purity) as a white solid. MS ES⁺: 295.2.

Step 2: A mixture of (1r,4r)—N¹,N¹-dibenzylcyclohexane-1,4-diamine hydrochloride (1.50 g, 4.53 mmol) in CH₃CN (15 mL) was treated with K₂CO₃ (1.25 g, 9.07 mmol) and 1-fluoro-2-iodo-ethane (789 mg, 4.53 mmol), stirred at 45° C. for 16 h and filtered. The filtrate was concentrated under reduced pressure to give a residue, which was purified by column chromatography (ISCO®; 40 g SepaFlash® Silica Flash Column, dichloromethane/MeOH: 1/0 to 10/1) to give (1r,4r)—N¹,N¹-dibenzyl-N⁴-(2-fluoroethyl)cyclohexane-1,4-diamine (1.3 g, 72.4% yield, 86% purity) as a yellow oil. ¹H NMR (400 MHz, DMSO-d₆): 7.38-7.25 (m, 8H), 7.22-7.16 (m, 2H), 4.45 (t, J=5.2 Hz, 1H), 4.33 (t, J=5.2 Hz, 1H), 3.56 (s, 4H), 2.79 (t, J=5.2 Hz, 1H), 2.72 (t, J=5.2 Hz, 1H), 2.43-2.24 (m, 2H), 1.94-1.75 (m, 4H), 1.47-1.31 (m, 2H), 0.90-0.76 (m, 2H). MS ES⁺: 341.4.

Step 3: A mixture of (1r,4r)—N¹,N¹-dibenzyl-N⁴-(2-fluoroethyl)cyclohexane-1,4-diamine (1.30 g, 3.82 mmol) in MeOH (15 mL) was treated with Pd(OH)₂ (600 mg, 0.854 mmol, 20% purity), degassed and purged with H₂ three times, stirred at 50° C. for 12 h under H₂ atmosphere (40 psi) and filtered. The filtrate was concentrated under reduced pressure to give (1r,4r)—N¹-(2-fluoroethyl)cyclohexane-1,4-diamine (550 mg, 90%) as a yellow oil. ¹H NMR (400 MHz, DMSO-d₆): 4.47 (t, J=5.2 Hz, 1H), 4.35 (t, J=5.2 Hz, 1H), 3.42-3.33 (m, 1H), 2.81 (t, J=5.3 Hz, 1H), 2.74 (t, J=5.3 Hz, 1H), 2.54-2.51 (m, 1H), 2.34-2.25 (m, 1H), 1.81 (d, J=10.3 Hz, 2H), 1.72 (d, J=10.4 Hz, 2H), 1.08-0.92 (m, 4H).

Step 4: A mixture of 2-(1H-imidazol-1-yl)-6-methyl-pyrimidine-4-carboxylic acid (212 mg, 0.624 mmol, 60% purity) and (1r,4r)-N¹-(2-fluoroethyl)cyclohexane-1,4-diamine (100 mg, 0.624 mmol) in CH₂Cl₂ (2.5 mL) was treated with NEt₃ (189 mg, 1.87 mmol) followed by dropwise addition of T₃P (476 mg, 0.749 mmol, 50% purity in EtOAc), stirred at 25° C. for 2 h and extracted with CH₂Cl₂ (3 mL). The combined organic layers were dried (Na₂SO₄), filtered, concentrated and purified by prep-HPLC (column: Xtimate C18 100×30 mm 10 μm, mobile phase A: H₂O (0.225% HCOOH), mobile phase B: CH₃CN, 0-20% B, flow rate: 25 mL/min). The product was partitioned between CH₃CN (2 mL) and H₂O (10 mL) and lyophilized to dryness to give the title compound (26.3 mg, 10.7%) as a white powder. ¹H NMR (400 MHz, DMSO-d₆): 8.93 (s, 1H), 8.80 (d, J=8.6 Hz, 1H), 8.24-8.17 (m, 2H), 7.82 (s, 1H), 7.16 (s, 1H), 4.63-4.41 (m, 2H), 3.81 (ddd, J=4.1, 7.9, 11.7 Hz, 1H), 3.00-2.88 (m, 2H), 2.62 (s, 3H), 2.53-2.52 (m, 1H), 1.99 (d, J=11.6 Hz, 2H), 1.84 (d, J=11.1 Hz, 2H), 1.63-1.48 (m, 2H), 1.27-1.12 (m, 2H). MS ES⁺: 347.4. UPLC purity: 99.4%. SFC chiral purity: 98.9%.

### Example 10: N-((1r,4r)-4-(3,3-difluoropyrrolidin-1-yl)cyclohexyl)-2-(1H-imidazol-1-yl)-6-methyl-pyrimidine-4-carboxamide

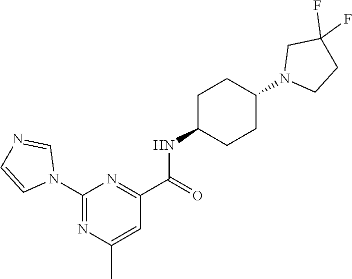 [SMILES: Cc1cc(C(=O)N[C@H]2CC[C@H](N3CCC(F)(F)C3)CC2)nc(-n2ccnc2)n1]

A solution of (1r,4r)-4-(3,3-difluoropyrrolidin-1-yl)-cyclohexan-1-amine (Intermediate 6) (50.0 mg, 0.196 mmol, 80% purity) and 2-(1H-imidazol-1-yl)-6-methyl-pyrimidine-4-carboxylic acid (57.1 mg, 0.196 mmol, 70% purity) in CH₂Cl₂ (0.5 mL) was treated with NEt₃ (59.5 mg, 0.588 mmol) followed by dropwise addition of T₃P (149.5 mg, 50% purity in EtOAc), stirred at 25° C. for 2 h and extracted with CH₂Cl₂ (3 mL×3). The combined organic layers were washed with brine, dried (Na₂SO₄), filtered and concentrated under reduced pressure and purified by prep-HPLC (column: Xtimate C18 100×30 mm 10 μm, mobile phase A: H₂O (0.225% HCOOH), mobile phase B: CH₃CN, 0-20% B, flow rate: 25 mL/min). The product was partitioned between CH₃CN (20 mL) and H₂O (100 mL) and lyophilized to dryness to give the title compound (37 mg, 21%) as a white powder. ¹H NMR (400 MHz, DMSO-d₆): 8.94 (s, 1H), 8.81 (d, J=8.6 Hz, 1H), 8.19 (s, 1H), 7.82 (s, 1H), 7.16 (s, 1H), 3.87-3.76 (m, 1H), 2.95 (t, J=13.7 Hz, 2H), 2.75 (t, J=6.8 Hz, 2H), 2.62 (s, 3H), 2.28-2.15 (m, 2H), 2.11 (s, 1H), 1.97 (d, J=12.3 Hz, 2H), 1.83 (d, J=10.8 Hz, 2H), 1.61-1.48 (m, 2H), 1.30-1.18 (m, 2H). MS ES⁺: 391.0. UPLC purity: 98.4%. SFC chiral purity: 100%.

### Example 11: N-((1s,4s)-4-(3,3-difluoropyrrolidin-1-yl)cyclohexyl)-2-(1H-imidazol-1-yl)-6-methyl-pyrimidine-4-carboxamide

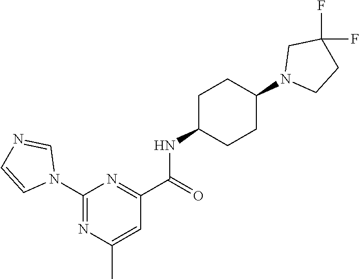 [SMILES: Cc1cc(C(=O)N[C@H]2CC[C@@H](N3CCC(F)(F)C3)CC2)nc(-n2ccnc2)n1]

A mixture of (1s,4s)-4-(3,3-difluoropyrrolidin-1-yl)-cyclohexan-1-amine (Intermediate 7) (120 mg, 0.529 mmol) and 2-(1H-imidazol-1-yl)-6-methyl-pyrimidine-4-carboxylic acid (180 mg, 0.529 mmol, 60% purity) in CH₂Cl₂ (0.5 mL) was treated with NEt₃ (161 mg, 1.59 mmol) followed by dropwise addition of T₃P (404 mg, 0.634 mmol, 50% purity in EtOAc), stirred at 25° C. for 2 h and extracted with CH₂Cl₂ (9 mL×3). The combined organic layers were dried (Na₂SO₄), filtered, concentrated under reduced pressure and purified by prep-HPLC (column: Xtimate C18 100×30 mm 10 μm, mobile phase A: H₂O (0.225% HCOOH), mobile phase B: CH₃CN, 0-20% B, flow rate: 25 mL/min). The product was partitioned between CH₃CN (20 mL) and H₂O (100 mL) and lyophilized to dryness to give the title compound (65.0 mg, 0.166 mmol, 31.5%) as a white powder. ¹H NMR (400 MHz, DMSO-d₆): 8.99-8.86 (m, 2H), 8.23-8.13 (m, 1H), 7.81 (s, 1H), 7.14 (s, 1H), 3.94-3.85 (m, 1H), 2.92 (t, J=14.1 Hz, 2H), 2.72 (t, J=6.9 Hz, 2H), 2.61 (s, 3H), 2.34-2.19 (m, 3H), 1.90-1.74 (m, 4H), 1.58-1.43 (m, 4H). MS ES⁺: 391.4. UPLC purity: 100%. SFC chiral purity: 100%.

### Example 12: N-((1s,4r)-4-((S)-3-fluoropyrrolidin-1-yl)cyclohexyl)-2-(1H-imidazol-1-yl)-6-methyl-pyrimidine-4-carboxamide

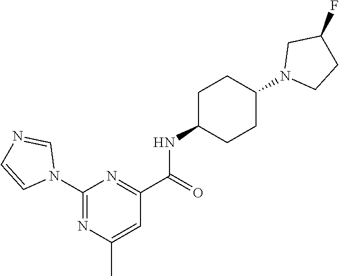 [SMILES: Cc1cc(C(=O)N[C@H]2CC[C@H](N3CC[C@H](F)C3)CC2)nc(-n2ccnc2)n1]

Step 1: A solution of 4-(dibenzylamino)cyclohexanone (Intermediate 4) (500 mg, 1.70 mmol), (3S)-3-fluoropyrrolidine hydrochloride (214 mg, 1.70 mmol) and acetic acid (471 mg, 1.70 mmol) in CH₂Cl₂ (5 mL) was stirred at 25° C. for 1 h, treated with NaBH(OAc)₃ (1.08 g, 5.11 mmol) and stirred at 25° C. for 4 h. The pH was adjusted to around 8 by addition of sat. NaHCO₃ (aq.) and the mixture was extracted with EtOAc (30 mL×3). The combined organic layers were washed with brine (30 mL), dried (Na₂SO₄), filtered, concentrated under reduced pressure and purified by flash silica gel chromatography (ISCO®; 12 g SepaFlash® Silica Flash Column, EtOAc/petroleum ether: 0 to 40%, 40 mL/min) to afford (1s,4r)—N,N-dibenzyl-4-((S)-3-fluoropyrrolidin-1-yl)-cyclohexan-1-amine (190 mg, 30%) as a white solid. ¹H NMR (400 MHz, DMSO-d₆): 7.15-7.38 (m, 10H), 5.01-5.23 (m, 1H), 3.57 (s, 4H), 2.69-2.85 (m, 2H), 2.53-2.69 (m, 1H), 2.26-2.44 (m, 2H), 1.99-2.12 (m, 2H), 1.88-1.95 (m, 2H), 1.70-1.87 (m, 3H), 1.31-1.47 (m, 2H), 0.91-1.05 (m, 2H).

Step 2: A mixture of (1s,4r)—N,N-dibenzyl-4-((S)-3-fluoropyrrolidin-1-yl)-cyclohexan-1-amine (190 mg, 0.518 mmol) in EtOH (2 mL) was treated with Pd(OH)₂ (190 mg, 0.271 mmol, 20% purity), degassed and purged with H₂ three times, and stirred at 50° C. for 12 h under H₂ atmosphere (40 psi). The mixture was filtered and concentrated under reduced pressure to give (1s,4r)-4-((S)-3-fluoropyrrolidin-1-yl)-cyclohexan-1-amine (105 mg, crude) as a white solid which was used for the next step without purification.

Step 3: A solution of (1s,4r)-4-((S)-3-fluoropyrrolidin-1-yl)-cyclohexan-1-amine (100 mg, 0.537 mmol) and 2-(1H-imidazol-1-yl)-6-methyl-pyrimidine-4-carboxylic acid (131 mg, 0.644 mmol) in CH₂Cl₂ (2 mL) was treated with DIPEA (208 mg, 1.61 mmol) followed by dropwise addition of T₃P (512 mg, 0.805 mmol, 50% purity in EtOAc), stirred at 25° C. for 1 h and extracted with CH₂Cl₂ (5 mL×3). The combined organic layers were dried (Na₂SO₄), filtered, concentrated under reduced pressure and purified by prep-HPLC (Column: Phenomenex C18 75×30 mm 3 μm, mobile phase A: H₂O (0.05% NH₃H₂O+10 mM NH₄HCO₃), mobile phase B: CH₃CN, 18-48% B, flow rate: 25 mL/min). The product was partitioned between CH₃CN (2 mL) and H₂O (10 mL) and lyophilized to dryness to give the title compound (28 mg, 14%) as a white solid. ¹H NMR (400 MHz, DMSO-d₆): 8.94 (s, 1H), 8.80 (d, J=8.6 Hz, 1H), 8.20 (s, 1H), 7.83 (s, 1H), 7.16 (s, 1H), 5.07-5.31 (m, 1H), 3.74-3.90 (m, 1H), 2.78-2.94 (m, 2H), 2.67-2.74 (m, 1H), 2.62 (s, 3H), 2.35-2.41 (m, 1H), 1.95-2.15 (m, 4H), 1.79-1.94 (m, 3H), 1.47-1.65 (m, 2H), 1.22-1.32 (m, 2H). MS ES⁺: 373.1. UPLC purity: 99.6%. SFC chiral purity: 100%.

### Example 13: N-((1r,4r)-4-((R)-3-fluoropyrrolidin-1-yl)cyclohexyl)-2-(1H-imidazol-1-yl)-6-methyl-pyrimidine-4-carboxamide

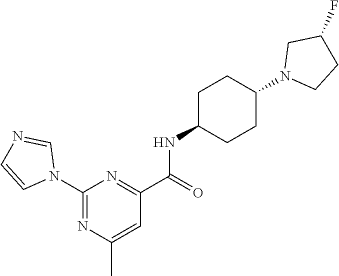 [SMILES: Cc1cc(C(=O)N[C@H]2CC[C@H](N3CC[C@@H](F)C3)CC2)nc(-n2ccnc2)n1]

Prepared as described for Example 12 using 2-(1H-imidazol-1-yl)-6-methyl-pyrimidine-4-carboxylic acid (100 mg, 0.343 mmol, 70% purity) and (1r,4r)-4-((R)-3-fluoropyrrolidin-1-yl)-cyclohexan-1-amine (70.2 mg, 0.377 mmol) to give the title compound (55 mg, 43%) as a white solid. ¹H NMR (400 MHz, DMSO-d₆): 8.94 (s, 1H), 8.80 (d, J=8.6 Hz, 1H), 8.19 (s, 1H), 7.82 (s, 1H), 7.16 (s, 1H), 5.34-5.01 (m, 1H), 3.91-3.72 (m, 1H), 2.93-2.78 (m, 2H), 2.74-2.64 (m, 1H), 2.62 (s, 3H), 2.42-2.34 (m, 1H), 2.15-1.94 (m, 4H), 1.94-1.76 (m, 3H), 1.62-1.47 (m, 2H), 1.28-1.22 (m, 2H). MS ES⁺:373.2. UPLC purity: 99.2%. SFC chiral purity: 99.8%.

### Example 14: 6-methyl-2-(1H-pyrazol-4-yl)-N-((1r,4r)-4-((2,2,2-trifluoroethyl)amino)cyclohexyl)pyrimidine-4-carboxamide

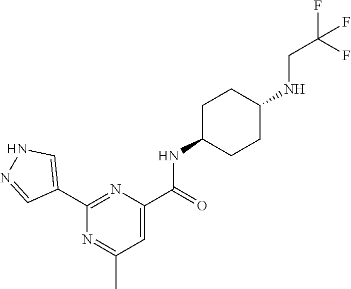 [SMILES: Cc1cc(C(=O)N[C@H]2CC[C@H](NCC(F)(F)F)CC2)nc(-c2cn[nH]c2)n1]

Step 1: A mixture of methyl 2-chloro-6-methyl-pyrimidine-4-carboxylate (500 mg, 2.68 mmol), 1-tetrahydropyran-2-yl-4-(4,4,5,5-tetramethyl-1,3,2-dioxaborolan-2-yl)pyrazole (894 mg, 3.22 mmol) and Cs₂CO₃ (1.75 g, 5.36 mmol) in H₂O (2 mL) and dioxane (8 mL) was treated with Pd(dppf)Cl₂—CH₂Cl₂ (219 mg, 0.268 mmol), stirred at 90° C. for 2 h under N₂, cooled to RT and filtered. The filtrate was concentrated to dryness to give 6-methyl-2-(1-(tetrahydro-2H-pyran-2-yl)-1H-pyrazol-4-yl)pyrimidine-4-carboxylic acid (810 mg, crude) as a black solid, which was used for the next step without further purification.

Step 2: A mixture of 6-methyl-2-(1-(tetrahydro-2H-pyran-2-yl)-1H-pyrazol-4-yl)pyrimidine-4-carboxylic acid (365 mg, 1.27 mmol) and (1r,4r)—N¹-(2,2,2-trifluoroethyl)cyclohexane-1,4-diamine (Intermediate 1) (299 mg, 1.52 mmol) in CH₂Cl₂ (4 mL) was treated with DIPEA (492 mg, 3.81 mmol) followed by dropwise addition of T₃P (1.62 g, 2.54 mmol, 50% of purity in EtOAc), stirred at 25° C. for 0.5 h and extracted with CH₂Cl₂ (40 mL×3). The combined organic layers were dried (Na₂SO₄) and filtered. The filtrate was evaporated to dryness to afford 6-methyl-2-(1-(tetrahydro-2H-pyran-2-yl)-1H-pyrazol-4-yl)-N-(trans-4-((2,2,2-trifluoroethyl)amino)cyclohexyl)pyrimidine-4-carboxamide (420 mg, crude) as a black oil which was used for the next step without further purification.

Step 3: A mixture of 6-methyl-2-(1-(tetrahydro-2H-pyran-2-yl)-1H-pyrazol-4-yl)-N-(trans-4-((2,2,2-trifluoroethyl)amino)cyclohexyl)pyrimidine-4-carboxamide (420 mg, 0.90 mmol, crude) in CH₂Cl₂ (5 mL) was treated with 4 M HCl in dioxane (5 mL), stirred at 25° C. for 0.5 h, concentrated and purified by prep-HPLC (Column: Xtimate C18 150×30 mm 5 μm, mobile phase A: H₂O (10 mM NH₄HCO₃), mobile phase B: CH₃CN, 20-55% B, flow rate: 25 mL/min). The product was partitioned between CH₃CN (2 mL) and H₂O (10 mL) and lyophilized to dryness to give the title compound (111 mg, 32%) as a white solid. ¹H NMR (400 MHz, DMSO-d₆): 13.47-13.17 (m, 1H), 8.69 (d, J=8.8 Hz, 1H), 8.63-8.37 (m, 2H), 7.71-7.64 (m, 1H), 3.91-3.80 (m, 1H), 3.32 (dd, J=7.8, 10.1 Hz, 2H), 2.61 (s, 3H), 2.52 (dd, J=6.3, 10.5 Hz, 1H), 2.36-2.25 (m, 1H), 2.01 (d, J=11.3 Hz, 2H), 1.89 (d, J=10.1 Hz, 2H), 1.65-1.54 (m, 2H), 1.25-1.14 (m, 2H). MS ES⁺: 383.3. UPLC purity: 99.2%. SFC chiral purity: 100%.

### Example 15: N-((1r,4r)-4-((2,2-difluoroethyl)amino)cyclohexyl)-6-methyl-2-(1H-pyrazol-4-yl)pyrimidine-4-carboxamide

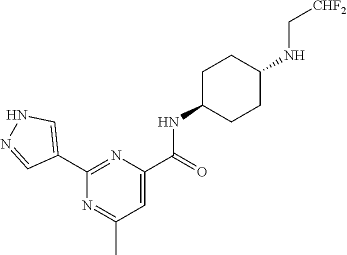 [SMILES: Cc1cc(C(=O)N[C@H]2CC[C@H](NCC(F)F)CC2)nc(-c2cn[nH]c2)n1]

Prepared as described for Example 14 using 6-methyl-2-(1-(tetrahydro-2H-pyran-2-yl)-1H-pyrazol-4-yl)pyrimidine-4-carboxylic acid (80 mg, 0.277 mmol) and (1r,4r)—N¹-(2,2-difluoroethyl)cyclohexane-1,4-diamine (Intermediate 2) (59 mg, 0.33 mmol) to give the title compound (25 mg, 24.2%) as a white solid. ¹H NMR (400 MHz, DMSO-d₆): 13.26 (s, 1H), 8.61 (d, J=8.4 Hz, 2H), 8.46-8.17 (m, 1H), 7.62 (s, 1H), 6.13-5.79 (m, 1H), 3.86-3.73 (m, 1H), 2.97-2.86 (m, 2H), 2.55 (s, 3H), 2.46-2.38 (m, 1H), 2.03-1.76 (m, 5H), 1.61-1.47 (m, 2H), 1.20-1.06 (m, 2H). MS ES⁺: 365.1. UPLC purity: 98.7%. SFC chiral purity: 100%.

### Example 16: 4-cyclopropyl-N-((1r,4r)-4-((2,2-difluoroethyl)amino)cyclohexyl)-6-(1H-imidazol-1-yl)picolinamide

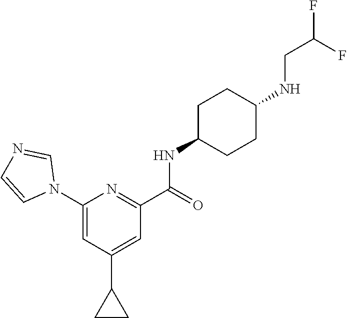 [SMILES: O=C(N[C@H]1CC[C@H](NCC(F)F)CC1)c1cc(C2CC2)cc(-n2ccnc2)n1]

Step 5: A solution of 4-cyclopropyl-6-(1H-imidazol-1-yl)picolinic acid (Intermediate 5) (150 mg, 0.654 mmol), (1r,4r)-N¹-(2,2-difluoroethyl)cyclohexane-1,4-diamine (Intermediate 2) (117 mg, 0.654 mmol) and NEt₃ (199 mg, 1.96 mmol) in CH₂Cl₂ (3 mL) was treated with HATU (373 mg, 0.982 mmol), stirred at 25° C. for 2 h and extracted with CH₂Cl₂ (20 mL×3). The combined organic layers were washed with brine (40 mL), dried (Na₂SO₄), filtered, concentrated under reduced pressure and purified by flash silica gel chromatography (ISCO®; 4 g SepaFlash® Silica Flash Column, MeOH/CH₂Cl₂: 0 to 3%, 40 mL/min) to give the title compound (60.6 mg, 24%) as a pale-yellow solid. ¹H NMR (400 MHz, DMSO-d₆): 8.91 (s, 1H), 8.49 (d, J=8.6 Hz, 1H), 8.24 (s, 1H), 7.64 (d, J=20.0 Hz, 2H), 7.13 (s, 1H), 5.95 (tt, J=4.4, 56.6 Hz, 1H), 3.87-3.71 (m, 1H), 2.91 (dt, J=4.4, 15.8 Hz, 2H), 2.40 (t, J=10.8 Hz, 1H), 2.18-2.10 (m, 1H), 1.97-1.76 (m, 5H), 1.59-1.47 (m, 2H), 1.19-1.13 (m, 2H), 1.13-1.05 (m, 2H), 1.02-0.96 (m, 2H). MS ES⁺: 390.2. UPLC purity: 97.1%. SFC chiral purity: 100%.

### Example 17 (comparative): N-((1r,4r)-4-(difluoromethoxy)cyclohexyl)-2-(1H-imidazol-1-yl)-6-methyl-pyrimidine-4-carboxamide

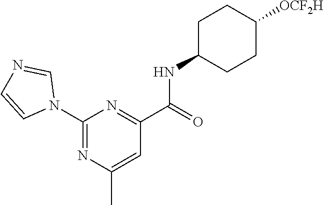

A solution of 2-(1H-imidazol-1-yl)-6-methyl-pyrimidine-4-carboxylic acid (100 mg, 0.245 mmol, 50% purity), (1r,4r)-4-(difluoromethoxy)cyclohexanamine (40.5 mg, 0.245 mmol) and NEt₃ (74.3 mg, 0.735 mmol) in CH₂Cl₂ (2 mL) was treated with T₃P (234 mg, 0.367 mmol, 50% purity in EtOAc), stirred at 25° C. for 5 h, extracted with CH₂Cl₂ (20 mL×3), washed with brine (40 mL), dried (Na₂SO₄), filtered, concentrated under reduced pressure and purified by prep-TLC (SiO₂, CH₂Cl₂/MeOH=20:1) to give the title compound (10.7 mg, 11.6%) as a white solid. ¹H NMR (400 MHz, DMSO-d₆): 8.93 (s, 1H), 8.80 (d, J=8.6 Hz, 1H), 8.19 (s, 1H), 7.83 (s, 1H), 7.17 (s, 1H), 6.95-6.56 (m, 1H), 4.11-4.00 (m, 1H), 3.88 (dd, J=3.4, 7.4 Hz, 1H), 2.63 (s, 3H), 2.04 (d, J=11.0 Hz, 2H), 1.92-1.84 (m, 2H), 1.69-1.48 (m, 4H). MS ES⁺: 352.1. UPLC purity: 93.3. SFC chiral purity: 98.1%.

### Example 18 (comparative): N-((1r,4r)-4-hydroxy-4-methylcyclohexyl)-2-(1H-imidazol-1-yl)-6-methyl-pyrimidine-4-carboxamide

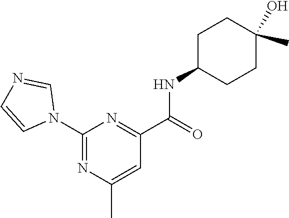 [SMILES: Cc1cc(C(=O)N[C@H]2CC[C@](C)(O)CC2)nc(-n2ccnc2)n1]

A mixture of 2-(1H-imidazol-1-yl)-6-methyl-pyrimidine-4-carboxylic acid (530 mg, 2.60 mmol), (1r,4r)-4-amino-1-methylcyclohexanol (402 mg, 3.11 mmol) and DIPEA (1.01 g, 7.79 mmol) in CH₂Cl₂ (5 mL) was treated with HATU (1.48 g, 3.89 mmol), stirred at 25° C. for 12 h, extracted with EtOAc (20 mL×3), washed with brine, dried (Na₂SO₄), filtered, concentrated under reduced pressure and purified by prep-HPLC (Column: Xtimate C18 150×40 mm 10 μm, mobile phase A: H₂O (0.05% NH₃— H₂O), mobile phase B: CH₃CN, 15-45% B, flow rate: 25 mL/min). The product was partitioned between CH₃CN (2 mL) and H₂O (10 mL) and lyophilized to dryness to give the title compound (6.8 mg, 1%) as a white solid. ¹H NMR (400 MHz, DMSO-d₆): 8.92 (s, 1H), 8.73 (d, J=8.4 Hz, 1H), 8.18 (s, 1H), 7.82 (s, 1H), 7.16 (s, 1H), 4.39 (s, 1H), 3.90-3.77 (m, 1H), 2.62 (s, 3H), 1.77-1.70 (m, 2H), 1.65-1.57 (m, 4H), 1.49 (dd, J=3.6, 13.2 Hz, 2H), 1.20 (s, 3H). MS ES⁺: 316.1. UPLC purity: 95.3%. SFC chiral purity: 100%.

### Example 19: N-((1r,4r)-4-(ethylamino)cyclohexyl)-2-(1H-imidazol-1-yl)-6-methyl-pyrimidine-4-carboxamide Formate

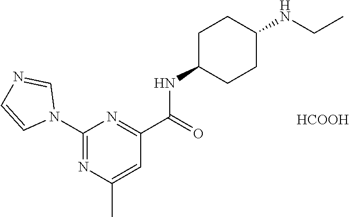 [SMILES: CCN[C@H]1CC[C@H](NC(=O)c2cc(C)nc(-n3ccnc3)n2)CC1 ; "HCOOH"]

To a solution of 2-(1H-imidazol-1-yl)-6-methyl-pyrimidine-4-carboxylic acid (239.25 mg, 0.703 mmol) and (1r,4r)—N¹-ethylcyclohexane-1,4-diamine (Intermediate 8) (100 mg, 0.703 mmol) in N,N-dimethylformamide (1 mL) was added triethylamine (213.42 mg, 2.11 mmol) followed by T₃P (537 mg, 0.844 mmol, 50% purity). The resulting mixture was stirred at 25° C. for 1 hour. The reaction mixture was diluted with H₂O (i mL) and then extracted with EtOAc (3×10 mL). The combined organic layers were washed with 4% LiCl (aq.) (10 mL), dried over Na₂SO₄, filtered and concentrated under reduced pressure to give a residue. The crude was purified by prep-HPLC (Column: Xtimate C18 100*30 mm*10 μm, Mobile Phase A: water(HCOOH), Mobile Phase B: acetonitrile, Flow rate: 25 mL/min, gradient condition from 0% to 30%). The pure fractions were collected and the volatiles were removed under vacuum. The residue was partitioned between acetonitrile (2 mL) and water (10 mL). The solution was lyophilized to dryness to give the residue. The residue was purified by prep. HPLC (Column: Xtimate C18 100*30 mm*10 μm, Mobile Phase A: water (HCOOH), Mobile Phase B: acetonitrile, Flow rate: 25 mL/min, gradient condition from 0% to 30%). The pure fractions were collected and the volatiles were removed under vacuum. The residue was partitioned between acetonitrile (2 mL) and water (10 mL). The solution was lyophilized to dryness to give the title compound (3.36 mg, 0.009 mmol, 1.9% yield) as a brown solid. ¹H NMR (400 MHz, DMSO-d₆) 10.30 (s, 1H), 9.18 (d, J=8.4 Hz, 1H), 8.99 (s, 2H), 8.62 (s, 1H), 8.04 (s, 1H), 7.81 (s, 1H), 3.86 (dd, J=3.6, 7.8 Hz, 1H), 2.98 (d, J=6.8 Hz, 3H), 2.69 (s, 3H), 2.17 (d, J=10.6 Hz, 2H), 1.91 (d, J=10.6 Hz, 2H), 1.71-1.61 (m, 2H), 1.53 (q, J=11.4 Hz, 2H), 1.24 (t, J=7.2 Hz, 3H). MS ES⁺: 329.4. UPLC purity: 97.4%. SFC chiral purity: 100%.

### Example 20: 4-cyclopropyl-6-(1H-imidazol-1-yl)-N-((1r,4r)-4-((2,2,2-trifluoroethyl)amino)cyclohexyl)picolinamide

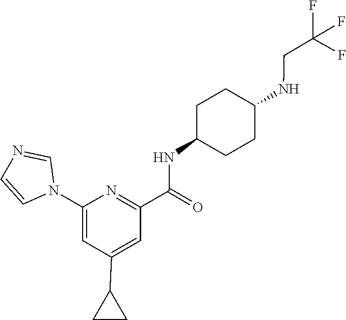 [SMILES: O=C(N[C@H]1CC[C@H](NCC(F)(F)F)CC1)c1cc(C2CC2)cc(-n2ccnc2)n1]

Step 1: To a solution of imidazole (1.24 g, 18.3 mmol) in THF (50 mL) was added NaH (8763 mg, 21.9 mmol, 60% purity) in portions at 0° C. After stirring for 0.5 hour, 2,6-dichloro-4-iodopyridine (5 g, 18.3 mmol) was added and the mixture stirred at 60° C. for 12 hours. The reaction mixture was quenched by addition sat. (aq.) NH₄Cl solution 50 mL at 0° C., and then diluted with H₂O (so mL) and extracted with EtOAc (3×50 mL). The combined organic layers were washed with brine (100 mL), dried over anhydrous Na₂SO₄, filtered and concentrated under reduced pressure to give a residue. The residue was purified by flash silica gel chromatography (ISCO®; 80 g SepaFlash® Silica Flash Column, Eluent of 0˜50% Ethyl acetate/Petroleum ether gradient @100 mL/min) to give 2-chloro-6-(1H-imidazol-1-yl)-4-iodopyridine (1.45 g, 4.75 mmol, 26.0% yield) as a yellow solid. ¹H NMR (400 MHz, DMSO-d₆) 8.53 (s, 1H), 8.33 (d, J=0.8 Hz, 1H), 7.96 (d, J=0.8 Hz, 1H), 7.95 (t, J=1.4 Hz, 1H), 7.13 (s, 1H).

Step 2: A mixture of 2-chloro-6-(1H-imidazol-1-yl)-4-iodopyridine (1.15 g, 3.76 mmol), cyclopropylboronic acid (647 mg, 7.53 mmol), K₃PO₄ (4.79 g, 22.6 mmol), tricyclohexylphosphonium tetrafluoroborate (277 mg, 0.753 mmol) and Pd(OAc)₂ (169 mg, 0.753 mmol) in H₂O (5 mL) and toluene (20 mL) was degassed and purged with N₂ three times. The mixture was stirred at 110° C. for 6 hours under N₂ atmosphere. The reaction mixture was concentrated under reduced pressure to remove the solvent to give a residue. The residue was purified by flash silica gel chromatography (ISCO®; 20 g SepaFlash® Silica Flash Column, Eluent of 035% Ethyl acetate/Petroleum ether gradient @80 mL/min) to give 2-chloro-4-cyclopropyl-6-(1H-imidazol-1-yl)pyridine (750 mg, 3.41 mmol, 90.7% yield) as a yellow solid. MS ES⁺: 220.0.

Step 3: A mixture of 2-chloro-4-cyclopropyl-6-(1H-imidazol-1-yl)pyridine (750 mg, 3.41 mmol), triethylamine (1.04 g, 10.24 mmol) and Pd(dppf)Cl₂ (250 mg, 0.341 mmol) in MeOH (10 mL) was degassed and purged with CO 3 times, and then the mixture was stirred at 80° C. for 16 hours under CO (50 psi) atmosphere. The reaction mixture was filtered and concentrated under reduced pressure to give a residue. The residue was purified by flash silica gel chromatography (ISCO®; 20 g SepaFlash® Silica Flash Column, Eluent of 0˜60% Ethyl acetate/Petroleum ether gradient @80 mL/min) to give methyl 4-cyclopropyl-6-imidazol-1-yl-pyridine-2-carboxylate (650 mg, 2.67 mmol, 78.3% yield) as a brown solid. ¹H NMR (400 MHz, CDCl₃) 8.50 (s, 1H), 7.72 (s, 1H), 7.66 (d, J=1.2 Hz, 1H), 7.29 (d, J=1.2 Hz, 1H), 7.23 (s, 1H), 4.00 (s, 3H), 2.09-2.01 (m, 1H), 1.28-1.22 (m, 2H), 1.00-0.94 (m, 2H).

Step 4: To a solution of methyl 4-cyclopropyl-6-(1H-imidazol-1-yl)picolinate (650 mg, 2.67 mmol) in THF (12 mL) was added a solution of LiOH·H₂O (336.38 mg, 8.02 mmol) in H₂O (4 mL). The mixture was stirred at 25° C. for 1 hour. The reaction mixture was acidized by 1N HCl (aq.) until pH=3-4, then concentrated under reduced pressure to give a residue. Crude 4-cyclopropyl-6-(1H-imidazol-1-yl)picolinic acid (1 g, crude), a grey solid, was used in the next step without further purification.

Step 5: To a solution of 4-cyclopropyl-6-(1H-imidazol-1-yl)picolinic acid (150 mg, 0.654 mmol) and (1r,4r)—N¹-(2,2,2-trifluoroethyl)cyclohexane-1,4-diamine (Intermediate 1) (128 mg, 0.654 mmol) in dichloromethane (2 mL) was added triethylamine (199 mg, 1.96 mmol), followed by T₃P (625 mg, 0.981 mmol, 50% purity). The resulting mixture was stirred at 25° C. for 2 hours. The reaction mixture was diluted with H₂O (10 mL) and extracted with dichloromethane (3×10 mL). The combined organic layers were dried over Na₂SO₄, filtered and concentrated under reduced pressure to give a residue. Then the product was further purified by prep. HPLC (Column: C18-6 100*30 mm*5 μm, Mobile Phase A: water (HCOOH), Mobile Phase B: acetonitrile, Flow rate: 25 mL/min, gradient condition from 5% to 35%). The pure fractions were collected and the volatiles were removed under vacuum. The residue was partitioned between acetonitrile (2 mL) and water (10 mL). The solution was lyophilized to dryness to give the title compound (73.46 mg, 0.180 mmol, 27.5% yield) as a white solid. ¹H NMR (400 MHz, DMSO-d₆) 8.91 (s, 1H), 8.50 (d, J=8.6 Hz, 1H), 8.24 (s, 1H), 7.66 (s, 1H), 7.61 (s, 1H), 7.12 (s, 1H), 3.87-3.70 (m, 1H), 3.25 (d, J=10.2 Hz, 3H), 2.45-2.37 (m, 1H), 2.19-2.08 (m, 1H), 1.99-1.88 (m, 2H), 1.79 (d, J=10.6 Hz, 2H), 1.53 (q, J=11.6 Hz, 2H), 1.19-1.08 (m, 4H), 0.99 (d, J=2.6 Hz, 2H). MS ES⁺:408.4. UPLC purity: 99.8%. SFC chiral purity: 100%.

### Example 21: 2-(1H-imidazol-1-yl)-6-methyl-N-((1r,4r)-4-((1,1,1-trifluoro-2-methylpropan-2-yl)amino)cyclohexyl)pyrimidine-4-carboxamide

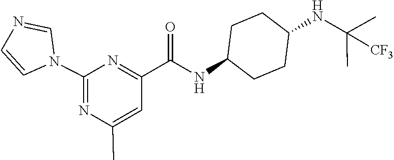 [SMILES: Cc1cc(C(=O)N[C@H]2CC[C@H](NC(C)(C)C(F)(F)F)CC2)nc(-n2ccnc2)n1]

To a solution of 2-(1H-imidazol-1-yl)-6-methyl-pyrimidine-4-carboxylic acid (109 mg, 0.535 mmol) and N¹-(1,1,1-trifluoro-2-methylpropan-2-yl)cyclohexane-1,4-diamine hydrochloride (Intermediate 9) (120 mg, 0.535 mmol) in N,N-dimethylformamide (3 mL) was added N-ethyl-N-isopropylpropan-2-amine (207 mg, 1.61 mmol) and HATU (305 mg, 0.803 mmol). The mixture was stirred at 25° C. for 1 hour. The reaction mixture was diluted with H₂O (50 mL) and extracted with dichloromethane (30 mL). The reaction mixture was washed with NaHCO₃ (3×30 mL). The combined organic layers were washed with brine (30 mL), dried over Na₂SO₄, filtered and concentrated under reduced pressure to give a residue. The residue was purified by prep. HPLC (Column: Phenomenex C18 75*30 mm*3 μm, Mobile Phase A: water (NH₃H₂O+NH₄HCO₃), Mobile Phase B: acetonitrile, Flow rate: 25 mL/min, gradient condition from 33% to 63%). The pure fractions were collected and the volatiles were removed under vacuum. The residue was partitioned between acetonitrile (2 mL) and water (10 mL). The solution was lyophilized to dryness to give the title compound (52.16 mg, 0.122 mmol, 22.8% yield) as a white solid. ¹H NMR (400 MHz, DMSO-d₆) 8.93 (t, J=1.0 Hz, 1H), 8.74 (d, J=8.8 Hz, 1H), 8.19 (t, J=1.4 Hz, 1H), 7.82 (s, 1H), 7.20-7.13 (m, 1H), 3.87-3.69 (m, 1H), 2.68-2.64 (m, 1H), 2.63 (s, 3H), 1.87-1.76 (m, 5H), 1.65-1.52 (m, 2H), 1.22 (s, 8H). MS ES⁺: 411.2. UPLC purity: 95.9%. SFC chiral purity: 100%.

### Example 22: 2-(1H-imidazol-1-yl)-6-methyl-N-((1S,4r)-4-(((S)-1,1,1-trifluoropropan-2-yl)amino)cyclohexyl)pyrimidine-4-carboxamide

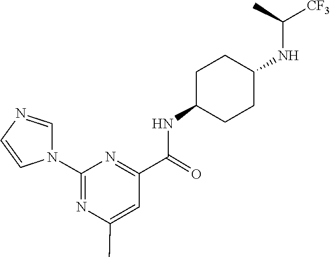 [SMILES: Cc1cc(C(=O)N[C@H]2CC[C@H](N[C@@H](C)C(F)(F)F)CC2)nc(-n2ccnc2)n1]

To a mixture of (1r,4S)-N¹-((S)-1,1,1-trifluoropropan-2-yl)cyclohexane-1,4-diamine (Intermediate 10) (100 mg, 0.476 mmol) and 2-(1H-imidazol-1-yl)-6-methyl-pyrimidine-4-carboxylic acid (80.93 mg, 0.396 mmol) in dichloromethane (3 mL) was added N-ethyl-N-isopropylpropan-2-amine (154 mg, 1.19 mmol), then added T₃P (504 mg, 0.793 mmol, 50% purity in EtOAc) at 25° C. The mixture was stirred at 25° C. for 0.5 hours. Water (15 mL) was added and the mixture was extracted with DCM (3×15 mL), and the organic phase was concentrated to the crude product. The product was purified by prep-HPLC (Column: Phenomenex C18 75*30 mm*3 μm, Mobile Phase A: water (NH₃H₂O+NH₄HCO₃), Mobile Phase B: acetonitrile, Flow rate: 25 mL/min, gradient condition from 25% B to 55%). The pure fractions were collected and the volatiles were removed under vacuum. The residue was partitioned between acetonitrile (2 mL) and water (10 mL). The solution was lyophilized to dryness to give the title compound (20.83 mg, 0.517 mmol, 13.0% yield) as a white solid. ¹H NMR (400 MHz, CDCl₃): 8.72 (s, 1H), 7.96-7.84 (m, 2H), 7.61 (br d, J=8.2 Hz, 1H), 7.24-7.16 (m, 1H), 4.02-3.90 (m, 1H), 3.28 (spt, J=6.9 Hz, 1H), 2.76-2.65 (m, 4H), 2.18-2.11 (m, 2H), 2.06-1.97 (m, 2H), 1.89 (s, 1H), 1.48-1.39 (m, 2H), 1.39-1.30 (m, 2H), 1.28-1.26 (m, 3H). MS ES⁺: 397.4. UPLC purity: 98.3%. SFC chiral purity: 100%.

### Example 23: 2-(1H-imidazol-1-yl)-6-methyl-N-((1R,4r)-4-(((R)-1,1,1-trifluoropropan-2-yl)amino)cyclohexyl)pyrimidine-4-carboxamide

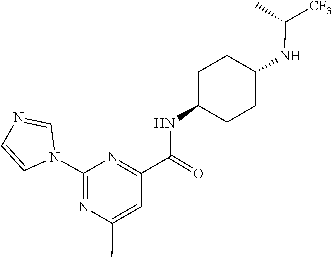 [SMILES: Cc1cc(C(=O)N[C@H]2CC[C@H](N[C@H](C)C(F)(F)F)CC2)nc(-n2ccnc2)n1]

A mixture of 2-(1H-imidazol-1-yl)-6-methyl-pyrimidine-4-carboxylic acid (43.7 mg, 0.214 mmol), (1r,4R)-N¹-((R)-1,1,1-trifluoropropan-2-yl)cyclohexane-1,4-diamine (Intermediate 11) (45 mg, 0.214 mmol), N-ethyl-N-isopropylpropan-2-amine (83.0 mg, 0.642 mmol) in dichloromethane (0.5 mL) was added T₃P (272 mg, 0.428 mmol, 50% purity in EtOAc) and then the mixture was stirred at 25° C. for 0.5 hour. The reaction mixture was diluted with H₂O (3 mL) and dichloromethane (3×3 mL). The aqueous phase was filtered to give a residue. The residue was purified by prep-HPLC (Column: Phenomenex C18 75*30 mm*3 μm, Mobile Phase A: water(NH₃H₂O+NH₄HCO₃), Mobile Phase B: acetonitrile, Flow rate: 25 mL/min, gradient condition from 25% B to 55%). The pure fractions were collected and the volatiles were removed under vacuum. The residue was partitioned between acetonitrile (2 mL) and water (10 mL). The solution was lyophilized to dryness to give the title compound (6.59 mg, 0.166 mmol, 7.7% yield) as an off-white powder. ¹H NMR (400 MHz, DMSO-d₆) 8.93 (s, 1H), 8.78 (d, J=8.8 Hz, 1H), 8.19 (t, J=1.3 Hz, 1H), 7.82 (s, 1H), 7.15 (s, 1H), 3.86-3.74 (m, 2H), 2.62 (s, 3H), 2.52 (br d, J=2.0 Hz, 1H), 2.02-1.78 (m, 6H), 1.54 (dq, J=3.1, 12.6 Hz, 2H), 1.16 (d, J=6.8 Hz, 3H). MS ES⁺: 397.4. UPLC purity: 99.7%. SFC chiral purity: 97.61%.

### Example 24: 6-methyl-2-(5-methyl-1H-imidazol-1-yl)-N-((1r,4r)-4-((2,2,2-trifluoroethyl)amino)cyclohexyl)pyrimidine-4-carboxamide

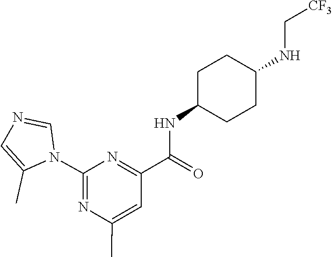 [SMILES: Cc1cc(C(=O)N[C@H]2CC[C@H](NCC(F)(F)F)CC2)nc(-n2cncc2C)n1]

Step 1: To a solution of methyl 2-chloro-6-methyl-pyrimidine-4-carboxylate (1 g, 5.36 mmol) and 5-methyl-1H-imidazole (440.01 mg, 5.36 mmol) in N,N-dimethylformamide (10 mL) was added N-ethyl-N-isopropylpropan-2-amine (2.08 g, 16.1 mmol) at 25° C. The mixture was stirred at 100° C. for 12 hours. The mixture was poured into H₂O (10 mL) and extracted with EtOAc (3×20 mL) and the combined organic layers were concentrated to afford a residue. The residue was purified by flash column chromatography (ISCO®; 20 g SepaFlash® Silica Flash Column, Eluent of 0-50% Ethyl acetate/Petroleum ether gradient @60 mL/min) to give methyl 6-methyl-2-(5-methyl-1H-imidazol-1-yl)pyrimidine-4-carboxylate (0.1 g, 0.415 mmol, 7.7% yield, 96.3% chemical purity) as a yellow solid. ¹H NMR (400 MHz, DMSO-d₆) 8.43 (s, 1H), 7.84 (s, 1H), 7.67-7.61 (m, 1H), 3.95 (s, 3H), 2.63 (s, 3H), 2.50 (br s, 3H).

Step 2: A mixture of methyl 6-methyl-2-(5-methyl-1H-imidazol-1-yl)pyrimidine-4-carboxylate (40 mg, 0.172 mmol) and 1 M LiOH (aq.) (1 M, 0.861 mL) in THF (2 mL) was stirred at 25° C. for 0.5 hour. The mixture was concentrated to afford the crude product. The crude product 6-methyl-2-(5-methyl-1H-imidazol-1-yl)pyrimidine-4-carboxylic acid (36 mg, crude), a yellow solid, was used in the next step without further purification. MS ES⁺: 219.0.

Step 3: To a mixture of 6-methyl-2-(5-methyl-1H-imidazol-1-yl)pyrimidine-4-carboxylic acid (28 mg, 0.128 mmol) and (1r,4r)-N¹-(2,2,2-trifluoroethyl)cyclohexane-1,4-diamine (Intermediate 1) (252 mg, 1.28 mmol) in dichloromethane (0.5 mL) was added triethylamine (130 mg, 1.28 mmol) and T₃P (306 mg, 0.962 mmol) in one portion at 25° C. The mixture was stirred at 25° C. for 0.5 hour. The mixture was poured into H₂O (5 mL) and extracted with dichloromethane (3×5 mL) and the combined organic layers were concentrated to afford the crude product. The residue was purified by prep-TLC (SiO₂, PE:EA=0:1, Rf=0.5) to give the crude product. The crude product was purified by prep. HPLC (DAICEL CHIRALCEL OD (250 mm*30 mm, 10 μm); Mobile Phase A [0.1% NH₃H₂O EtOH]; Mobile Phase B: acetonitrile, Flow rate: 25 mL/min, gradient condition from 30% B to 30%). The pure fractions were collected and the volatiles were removed under vacuum. The residue was partitioned between acetonitrile (2 mL) and water (10 mL). The solution was lyophilized to dryness to give the title compound (12.8 mg, 0.031 mmol, 46.8% yield) as a white solid. ¹H NMR (400 MHz, DMSO-d₆) 8.86 (d, J=1.1 Hz, 1H), 8.63 (d, J=8.5 Hz, 1H), 7.82 (s, 1H), 6.85 (t, J=1.1 Hz, 1H), 3.89-3.71 (m, 1H), 3.26 (br dd, J=7.9, 10.3 Hz, 2H), 2.62 (s, 3H), 2.56 (d, J=0.9 Hz, 3H), 2.24 (br d, J=6.4 Hz, 1H), 1.94 (br d, J=11.4 Hz, 2H), 1.82 (br d, J=10.4 Hz, 2H), 1.61-1.41 (m, 2H), 1.13 (br d, J=13.8 Hz, 2H). MS ES⁺: 397.2. UPLC purity: 95.4%. SFC chiral purity: 100%.

### Example 25: 2-(1H-imidazol-1-yl)-6-methyl-N-((1r,4r)-4-(((2,2,2-trifluoroethyl)amino)methyl)cyclohexyl)pyrimidine-4-carboxamide

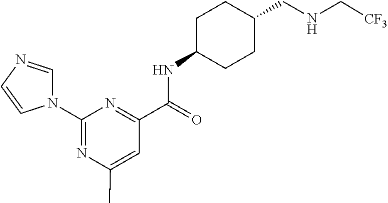 [SMILES: Cc1cc(C(=O)N[C@H]2CC[C@H](CNCC(F)(F)F)CC2)nc(-n2ccnc2)n1]

Step 1: To a solution of 2-imidazol-1-yl-6-methyl-pyrimidine-4-carboxylic acid (50 mg, 0.245 mmol), tert-butyl(((1r,4r)-4-aminocyclohexyl)methyl)carbamate (55.91 mg, 0.245 mmol), N-ethyl-N-isopropylpropan-2-amine (94.9 mg, 0.735 mmol) and dichloromethane (1 mL) was added T₃P (233.7 mg, 0.367 mmol, 50% purity in EtOAc). The mixture was stirred at 25° C. for 1 hour. The reaction mixture was diluted with H₂O (20 mL) and extracted with EtOAc (3×20 mL). The combined organic layers were dried over Na₂SO₄, filtered and concentrated under reduced pressure to give a residue. The crude product tert-butyl(((1r,4r)-4-(2-(1H-imidazol-1-yl)-6-methyl-pyrimidine-4-carboxamido)cyclohexyl)methyl)carbamate (102 mg, crude), a yellow solid, was used in the next step without further purification. MS ES⁺: 415.2.

Step 2: To a solution of tert-butyl(((1r,4r)-4-(2-(1H-imidazol-1-yl)-6-methyl-pyrimidine-4-carboxamido)cyclohexyl)methyl)carbamate (100 mg, 0.241 mmol) in dichloromethane (1 mL) was added 2,2,2-trifluoroacetic acid (308 mg, 2.70 mmol). The mixture was stirred at 25° C. for 1 hour. The reaction mixture was filtered and concentrated under reduced pressure to give a residue. The crude product N-((1r,4r)-4-(aminomethyl)cyclohexyl)-2-(1H-imidazol-1-yl)-6-methyl-pyrimidine-4-carboxamide trifluoroacetate (105 mg, crude), a yellow solid, was used in the next step without further purification. MS ES⁺: 315.1. Step 3: To a solution of N-((1r,4r)-4-(aminomethyl)cyclohexyl)-2-(1H-imidazol-1-yl)-6-methyl-pyrimidine-4-carboxamide trifluoroacetate (105 mg, 0.245 mmol), triethylamine (124 mg, 1.23 mmol) and N,N-dimethylformamide (1 mL) was added 2,2,2-trifluoroethyl trifluoromethanesulfonate (56.9 mg, 0.245 mmol). The mixture was stirred at 70° C. for 12 hours. The reaction mixture was filtered and concentrated under reduced pressure to give a residue. Then the product was further purified by prep. HPLC (Column: Welch Xtimate C18 150*30 mm*5 μm, Mobile Phase A: water (NH₄HCO₃), Mobile Phase B: acetonitrile, Flow rate: 25 mL/min, gradient condition from 27% B to 68%). The pure fractions were collected and the volatiles were removed under vacuum. The residue was partitioned between acetonitrile (2 mL) and water (10 mL). The solution was lyophilized to dryness to give the title compound (3.77 mg, 0.009 mmol, 3.7% yield) as a yellow solid. ¹H NMR (400 MHz, DMSO-d₆) 8.94 (s, 1H), 8.81 (d, J=8.8 Hz, 1H), 8.22-8.18 (m, 1H), 7.82 (s, 1H), 7.15 (s, 1H), 3.86-3.76 (m, 1H), 3.24-3.17 (m, 2H), 2.62 (s, 3H), 2.53-2.51 (m, 2H), 2.32-2.23 (m, 1H), 1.88-1.80 (m, 4H), 1.54-1.45 (m, 2H), 1.40-1.32 (m, 1H), 1.03-0.94 (m, 2H). MS ES⁺: 397.2. UPLC purity: 95.3%. SFC chiral purity: 100%.

### Example 26: 2-(1H-imidazol-1-yl)-6-methyl-N-((1r,4r)-4-((2,2,2-trifluoroethyl)carbamoyl)cyclohexyl)pyrimidine-4-carboxamide

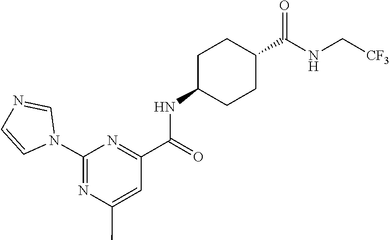 [SMILES: Cc1cc(C(=O)N[C@H]2CC[C@H](C(=O)NCC(F)(F)F)CC2)nc(-n2ccnc2)n1]

Step 1: To a solution of 2-(1H-imidazol-1-yl)-6-methyl-pyrimidine-4-carboxylic acid (2 g, 5.58 mmol) and methyl (1r,4r)-4-aminocyclohexane-1-carboxylate hydrochloride (1.19 g, 6.14 mmol) in dichloromethane (20 mL) was added N-ethyl-N-isopropylpropan-2-amine (2.16 g, 16.8 mmol) and T₃P (5.33 g, 8.37 mmol, 50% purity in EtOAc). The mixture was stirred at 25° C. for 1 hour. The reaction mixture was diluted with H₂O (20 mL) and extracted with dichloromethane (3×20 mL). The combined organic layers were dried over Na₂SO₄, filtered and concentrated under reduced pressure to give a residue. The residue was purified by column chromatography (SiO₂, Petroleum ether:Ethyl acetate=1/0 to 0/1) to give methyl (1r,4r)-4-(2-(1H-imidazol-1-yl)-6-methyl-pyrimidine-4-carboxamido)cyclohexane-1-carboxylate (400 mg, 1.16 mmol, 20.9% yield) as a yellow solid. MS ES⁺: 343.9.

Step 2: To a solution of methyl (1r,4r)-4-(2-(1H-imidazol-1-yl)-6-methyl-pyrimidine-4-carboxamido)cyclohexane-1-carboxylate (200 mg, 0.582 mmol) in THF (2 mL) was added 1 M LiOH (aq.) (1 M, 1.75 mL). The mixture was stirred at 25° C. for 1 hour. The mixture was adjusted pH with the addition of 1 M HCl (aq.) to 5-6. The mixture was concentrated under reduced pressure to give the crude product. The crude product (1r,4r)-4-(2-(1H-imidazol-1-yl)-6-methyl-pyrimidine-4-carboxamido)cyclohexane-1-carboxylic acid (236 mg, 0.573 mmol, 98.4% yield), an off-white solid, was used in the next step without further purification. MS ES⁺: 330.0.

Step 3: To a solution of (1r,4r)-4-(2-(1H-imidazol-1-yl)-6-methyl-pyrimidine-4-carboxamido)cyclohexane-1-carboxylic acid (50 mg, 0.121 mmol), 2,2,2-trifluoroethan-1-amine (13.2 mg, 0.134 mmol) in dichloromethane (0.5 mL) was added N-ethyl-N-isopropylpropan-2-amine (47.1 mg, 0.364 mmol) and T₃P (116 mg, 0.182 mmol, 50% purity in EtOAc). The mixture was stirred at 25° C. for 1 hour. The reaction mixture was concentrated under reduced pressure to give a residue. The residue was purified by prep. HPLC (Column: Welch Xtimate C18 100*30 mm*10 μm, Mobile Phase A: water (HCOOH), Mobile Phase B: acetonitrile, Flow rate: 25 mL/min, gradient condition from 5% to 35%). The pure fractions were collected and the volatiles were removed under vacuum. The residue was partitioned between acetonitrile (2 mL) and water (10 mL). The solution was lyophilized to dryness to give the title compound (7.21 mg, 0.017 mmol, 14.3% yield) as a white solid. ¹H NMR (400 MHz, DMSO-d₆) 9.05 (s, 1H), 8.84 (d, J=8.6 Hz, 1H), 8.49 (t, J=6.3 Hz, 1H), 8.24 (s, 1H), 7.85 (s, 1H), 7.22 (s, 1H), 3.95-3.86 (m, 2H), 3.85-3.80 (m, 1H), 2.63 (s, 3H), 2.27-2.18 (m, 1H), 1.88-1.80 (m, 3H), 1.57-1.46 (m, 4H), 0.98-0.91 (m, 1H). MS ES⁺: 411.1. UPLC purity: 98.8%. SFC chiral purity: 98.34%.

### Example 27: N-((1r,4r)-4-aminocyclohexyl)-2-(1H-imidazol-1-yl)-6-methyl-pyrimidine-4-carboxamide Formate

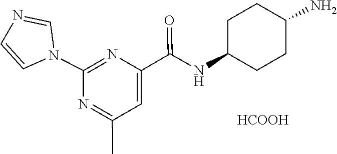 [SMILES: Cc1cc(C(=O)N[C@H]2CC[C@H](N)CC2)nc(-n2ccnc2)n1 ; "HCOOH"]

Step 1: To a solution of tert-butyl ((1r,4r)-4-aminocyclohexyl)carbamate (500 mg, 2.33 mmol), 2-(1H-imidazol-1-yl)-6-methyl-pyrimidine-4-carboxylic acid (476 mg, 2.33 mmol) and triethylamine (708 mg, 7.00 mmol) in dichloromethane (10 mL) was added T₃P (2.23 g, 3.50 mmol, 50% purity in EtOAc). The mixture was stirred at 25° C. for 1 hour. The reaction mixture was diluted with H₂O (20 mL) and then extracted with EtOAc (3×20 mL). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of Petroleum ether:Ethyl acetate=0:1@ 35 mL/min) to give tert-butyl ((1r,4r)-4-(2-(1H-imidazol-1-yl)-6-methyl-pyrimidine-4-carboxamido)cyclohexyl) carbamate (570 mg, 1.19 mmol, 51.1% yield) as light yellow solid. ¹H NMR (400 MHz, DMSO-d₆) 8.94 (s, 1H), 8.81 (d, 1H, J=8.6 Hz), 8.20 (t, 1H, J=1.2 Hz), 7.82 (s, 1H), 7.1-7.2 (m, 1H), 6.77 (d, 1H, J=7.8 Hz), 3.79 (dd, 1H, J=3.8, 7.8 Hz), 3.2-3.3 (m, 1H), 2.62 (s, 3H), 1.83 (t, 4H, J=12.2 Hz), 1.5-1.6 (m, 2H), 1.38 (s, 9H), 1.2-1.3 (m, 2H).

Step 2: To a solution of tert-butyl ((1r,4r)-4-(2-(1H-imidazol-1-yl)-6-methyl-pyrimidine-4-carboxamido)cyclohexyl)carbamate (50 mg, 0.125 mmol) in 4 M HCl in dioxane (1 mL) was added. The mixture was stirred at 25° C. for 0.5 hour. The reaction mixture was concentrated under reduced pressure to give a residue. The residue was purified by prep. HPLC (Column: Xtimate C18 100*30 mm*10 μm, Mobile Phase A: water (HCOOH), Mobile Phase B: acetonitrile, Flow rate: 25 mL/min, gradient condition from 0% to 20%). The pure fractions were collected and the volatiles were removed under vacuum. The residue was partitioned between acetonitrile (2 mL) and water (10 mL). The solution was lyophilized to dryness to give the title compound (18.09 mg, 0.520 mmol, 41.6% yield) as a white solid. ¹H NMR (400 MHz, DMSO-d₆) 8.92 (s, 1H), 8.82 (d, J=8.6 Hz, 1H), 8.42 (s, 1H), 8.18 (s, 1H), 7.82 (s, 1H), 7.16 (s, 1H), 3.89-3.75 (m, 1H), 3.30-3.22 (m, 2H), 2.97-2.81 (m, 1H), 2.62 (s, 3H), 2.02-1.94 (m, 2H), 1.90-1.82 (m, 2H), 1.64-1.50 (m, 2H), 1.45-1.33 (m, 2H). MS ES⁺: 301.4. UPLC purity: 99.5%. SFC chiral purity: 96.89%.

### Example 28: 2-(1H-imidazol-1-yl)-6-methyl-N-((1r,4r)-4-((2,2,2-trifluoroethyl-1,1-d₂)amino)cyclohexyl)pyrimidine-4-carboxamide

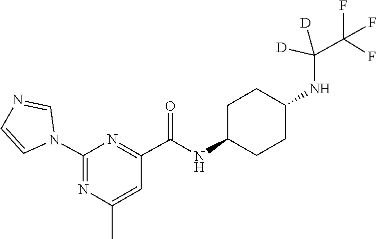 [SMILES: [2H]C([2H])(N[C@H]1CC[C@H](NC(=O)c2cc(C)nc(-n3ccnc3)n2)CC1)C(F)(F)F]

Step 1: To a solution of 2-(1H-imidazol-1-yl)-6-methylpyrimidine-4-carboxylic acid (4.84 g, 23.7 mmol), tert-butyl((trans)-4-aminocyclohexyl)carbamate (6.10 g, 28.44 mmol) and N-ethyl-N-isopropylpropan-2-amine (9.19 g, 71.11 mmol) in dichloromethane (50 mL) was added T₃P (22.63 g, 35.56 mmol, 50% purity in EtOAc). Then the mixture was stirred at 25° C. for 1 hour. The reaction mixture was diluted with 50 mL saturated NaHCO₃ and extracted with dichloromethane (3×50 mL). The combined organic layers were washed with brine (100 mL), dried over anhydrous Na₂SO₄, filtered and concentrated under reduced pressure to dryness. Purification by flash silica gel chromatography (ISCO®; 80 g SepaFlash® Silica Flash Column, Eluent: dichloromethane/methanol=20/1 @ 80 mL/min) gave tert-butyl ((1r,4r)-4-(2-(1H-imidazol-1-yl)-6-methylpyrimidine-4-carboxamido)cyclohexyl)carbamate (5.89 g, 13.02 mmol, 54.9% yield) as a white solid. ¹H NMR (400 MHz, DMSO-d₆) 8.94 (s, 1H), 8.82 (d, J=8.8 Hz, 1H), 8.20 (s, 1H), 7.82 (s, 1H), 7.16 (s, 1H), 6.78 (d, J=8.0 Hz, 1H), 3.88-3.68 (m, 1H), 3.26-3.20 (m, 1H), 2.62 (s, 3H), 1.83 (t, J=11.9 Hz, 4H), 1.62-1.51 (m, 2H), 1.38 (s, 9H), 1.32-1.22 (m, 2H).

Step 2: To a solution of tert-butyl ((1r,4r)-4-(2-(1H-imidazol-1-yl)-6-methylpyrimidine-4-carboxamido)cyclohexyl)carbamate (2 g, 4.99 mmol) in dichloromethane (10 mL) was added 4 M HCl in dioxane (4 M, 20 mL). The mixture was stirred at 25° C. for 1 hour. The reaction mixture was filtered and concentrated under reduced pressure to give N-((1r,4r)-4-aminocyclohexyl)-2-(1H-imidazol-1-yl)-6-methylpyrimidine-4-carboxamide hydrochloride (0.89 g, 1.97 mmol, 39.4% yield) as a white solid, which was used in the next step without further purification. ¹H NMR (400 MHz, DMSO-d₆) 10.23 (s, 1H), 9.13 (d, J=8.5 Hz, 1H), 8.60 (s, 1H), 8.10 (d, J=4.1 Hz, 3H), 8.03 (s, 1H), 7.79 (s, 1H), 3.88-3.83 (m, 1H), 2.99 (d, J=5.1 Hz, 1H), 2.69 (s, 3H), 2.05 (d, J=11.4 Hz, 2H), 1.89 (d, J=10.5 Hz, 2H), 1.72-1.59 (m, 2H), 1.56-1.42 (m, 2H).

Step 3: To a solution of N-((1r,4r)-4-aminocyclohexyl)-2-(1H-imidazol-1-yl)-6-methylpyrimidine-4-carboxamide hydrochloride (20 mg, 0.594 mmol) and 2,2,2-trifluoroethyl-1,1-d₂-4-methylbenzenesulfonate (Intermediate 12) (22.8 mg, 0.891 mmol) in DMF (0.2 mL) was added KI (9.86 mg, 0.594 mmol), K₂CO₃ (24.6 mg, 0.178 mmol) and 1,4,7,10,13,16-hexaoxacyclooctadecane (3.14 mg, 0.119 mmol). The mixture was stirred at 90° C. for 1 hour. The combined reaction mixture was poured into sat. KHCO₃ aq. (10 mL) and extracted with ethyl acetate (3×20 mL). The combined organic layers were washed with brine (50 mL), dried over Na₂SO₄ and filtered. The filtrate was evaporated to dryness. The product was purified by prep. HPLC (Column: Xtimate C18 150*40 mm*10 μm, Mobile Phase A: water (NH₃H₂O+NH₄HCO₃), Mobile Phase B: acetonitrile, Flow rate: 25 mL/min, gradient condition from 14% to 54%). The pure fractions were collected and the volatiles were removed under vacuum. The residue was partitioned between acetonitrile (2 mL) and water (10 mL). The solution was lyophilized to dryness to give the title compound (5 mg, 3.5% yield) as a yellow solid. ¹H NMR (400 MHz, DMSO-d₆) 8.96-8.91 (m, 1H), 8.82-8.76 (m, 1H), 8.21-8.17 (m, 1H), 7.84-7.80 (m, 1H), 7.17-7.14 (m, 1H), 3.88-3.76 (m, 1H), 2.63-2.61 (m, 3H), 2.45-2.41 (m, 1H), 2.24-2.20 (m, 1H), 1.99-1.92 (m, 2H), 1.84-1.78 (m, 2H), 1.58-1.48 (m, 2H), 1.17-1.08 (m, 2H). MS ES⁺: 385.1. UPLC purity: 97.5%. SFC chiral purity: 100%.

### Example 29 (comparative): 2-(1H-imidazol-1-yl)-N-((1r,4r)-4-((2,2,2-trifluoroethyl)amino)cyclohexyl)-5H-pyrrolo[3,2-d]pyrimidine-4-carboxamide

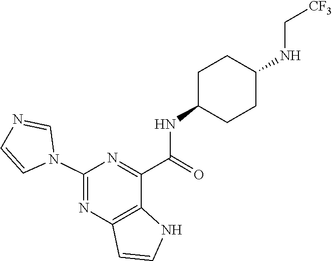 [SMILES: O=C(N[C@H]1CC[C@H](NCC(F)(F)F)CC1)c1nc(-n2ccnc2)nc2cc[nH]c12]

The compound of example 29 was prepared as described in example 101 in WO2021/021986 A1.

### Biological Activity

### Human CD38 Hydrolase Assay

The ability of test compounds to inhibit human CD38 hydrolase activity was measured in a fluorescence-based assay using non-physiological NAD⁺ substrate analogue 1,N⁶-etheno NAD⁺ (ε-NAD). Recombinant human CD38 (0.8 nM) was preincubated with test compounds in 384-well black microplates for 30 min at 25° C. in PBS (—Ca²⁺/Mg²⁺) containing 0.005% BSA (pH 7.4). CD38 hydrolase activity was initiated by addition of 4 μM ε-NAD, which yields the fluorescent product 1,N⁶-etheno ADP-ribose. Formation of fluorescent product was followed using ClarioStar Plus (BMG) microplate reader by reading fluorescence (excitation λ=300 nm; emission λ=410 nm) at two time points, one immediately after substrate addition (t=0) and one at 10 min (t=10).

Data was analysed by subtracting the values detected at t=0 from t=10 to correct for variation in baseline fluorescence. Fluorescence values were converted to percent inhibition using the average of high signal (CD38 and ε-NAD) and low signal (CD38 and ε-NAD in the presence of a tool CD38 inhibitor) control wells. IC₅₀ values were determined from a 10-point, half log concentration response curve with a four-parameter logistic equation.

### Mouse CD38 Hydrolase Assay

The ability of test compounds to inhibit mouse CD38 hydrolase activity was measured in a fluorescence-based assay using non-physiological NAD⁺ substrate analogue 1,N⁶-etheno NAD⁺ (ε-NAD). Recombinant mouse CD38 (0.4 nM) was preincubated with test compounds in 384-well black microplates for 30 min at 25° C. in PBS (—Ca²⁺/Mg²⁺) containing 0.005% BSA (pH 7.4). CD38 hydrolase activity was initiated by addition of 12 μM ε-NAD, which yields the fluorescent product 1,N⁶-etheno ADP-ribose. Formation of fluorescent product was followed using ClarioStar Plus (BMG) microplate reader by reading fluorescence (excitation λ=300 nm; emission λ=410 nm) at two time points, one immediately after substrate addition (t=0) and one at 6 min (t=6).

Data was analysed by subtracting the values detected at t=0 from t=6 to correct for variation in baseline fluorescence, and fluorescence values were converted to percent inhibition using the average of high signal (CD38 and ε-NAD) and low signal (CD38 and ε-NAD in the presence of a tool CD38 inhibitor) control wells. IC₅₀ values were determined from a 10-point, half log concentration response curve with a four-parameter logistic equation.

The data for human and mouse CD38 activity is summarized in Table 1.

TABLE 1

| | human CD38 | mouse CD38 |
|---|---|---|
| Example | IC₅₀ (μM) | IC₅₀ (μM) |
| 1 | ++++ | ++++ |
| 2 | ++++ | ++++ |
| 3 | ++++ | ++++ |
| 4 | ++++ | ++++ |
| 5 | ++ | ++++ |
| 6 | +++ | ++++ |
| 7 | +++ | ND |
| 8 | ++ | ND |
| 9 | ++ | ++++ |
| 10 | ++++ | ++++ |
| 11 | + | ND |
| 12 | +++ | ++++ |
| 13 | +++ | ++++ |
| 14 | + | ++ |
| 15 | + | ++ |
| 16 | ++ | ++++ |
| 19 | +++ | ++++ |
| 20 | +++ | ++++ |
| 21 | +++ | ++++ |
| 22 | ++++ | ++++ |
| 23 | ++++ | ++++ |
| 24 | +++ | ++++ |
| 25 | ++++ | ++++ |
| 26 | ++++ | ++++ |
| 27 | ++ | ++++ |

IC50 (≤10 μM = ‘+’; ≤1 μM = ‘++’; ≤0.1 μM = ‘+++’; ≤0.04 μM = ‘++++’; not determined = ‘ND’)

### Pharmacokinetics

### Tissue Binding Assays

Buffer preparation. A basic solution was prepared by dissolving 14.2 g/L Na₂HPO₄ and 8.77 g/L NaCl in deionized H₂O. An acidic solution was prepared by dissolving 15.6 g/L NaH₂PO₄·2H₂O and 8.77 g/L NaCl in deionized H₂O. Using the acidic solution, the basic solution was then titrated to pH 7.4±0.1 and stored at 4° C. for up to 1 month. The stop solution was 100% CH₃CN containing 200 ng/mL tolbutamide, 200 ng/mL labetalol and 50 ng/mL metformin.

Test method. Dialysis membrane strips were soaked in ultra-pure water at room temperature for ˜1 hour. Each membrane strip containing 2 membranes was separated and soaked in 20:80 EtOH/H₂O (v/v) for ˜20 min, after which they were ready for use or were stored in the solution at 2-8° C. for up to 1 month. Prior to the experiment, the membrane was rinsed and soaked for 20 min in ultra-pure water. On the day of experiment, brain homogenate was thawed in a water bath at room temperature and incubated at 37° C. for 10 min before use. Test and control compounds were dissolved in DMSO to achieve 10 mM stock solutions. DMSO working solutions were prepared at 400 μM by diluting 10 μL of stock solution. To prepare the time zero (t=0) samples to be used for recovery determination, 50 μL aliquots of loading matrix were transferred in triplicate to the sample collection plate. The samples were immediately matched with opposite blank buffer to obtain a final volume of 100 μL of 1:1 matrix/dialysis buffer (v/v) in each well. 500 μL of stop solution were added to these t=0 samples. They were then stored at 2-8° C. pending further processes along with other post-dialysis samples. To load the dialysis device, an aliquot of 150 μL of the loading matrix was transferred to the donor side of each dialysis well in triplicate, and 150 μL of the dialysis buffer was loaded to the receiver side of the well. The dialysis plate was placed in a humidified incubator at 37° C. with 5% CO₂ on a shaking platform that rotated slowly (about 100 rpm) for 4 hours. At the end of the dialysis, aliquots of 50 μL of samples were taken from both the buffer side and the matrix side of the dialysis device. These samples were transferred into new 96-well plates. Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 min. An aliquot of 100 μL of supernatant of all the samples was then removed for LC-MS/MS analysis. The single blank samples were prepared by transferring 50 μL of blank matrix to a 96-well plate and adding 50 μL of blank PBS buffer to each well. Then the matrix-matched samples were further processed by adding 500 μL of stop solution containing internal standards, following the same sample processing method as the dialysis samples.

Data analysis. The % undiluted unbound, % undiluted bound and % recovery were calculated using the following equations:

% Undiluted Unbound=100*1/D/((1/(F/T)−1)+1/D)

% Undiluted Bound=100−% Undiluted Unbound

% Recovery=100*(F+T)/To

where F is the analyte concentration or peak area ratio of analyte/internal standard on the buffer (receiver) side of the membrane; T is the analyte concentration or peak area ratio of analyte/internal standard on the matrix (donor) side of the membrane; To is the analyte concentration or the peak area ratio of analyte/internal standard in the loading matrix sample at time zero; and D is the dilution factor determined as 4 in this assay.

Unbound fractions in brain homogenate and plasma for selected compounds are summarized in Table 2.

TABLE 2

| | Mouse brain | Mouse plasma |
|---|---|---|
| Example | (% unbound) | (% unbound) |
| 1 | 68.9 | 13.0 |
| 2 | 63.4 | 22.7 |
| 3 | 13.5 | 7.4 |
| 10 | 9.7 | 10.6 |
| 17 | 13.6 | 6.9 |
| 18 | 28.4 | 25.3 |
| 21 | 3.6 | 4.1 |

### PK Brain Permeability

The distribution of compounds into the brain in vivo was determined in C57BL/6 mice following single oral (po) gavage administration.

Test compounds were formulated at 1 mg/mL in 0.5% HPMC E4 M, 0.2% Tween 80 in water to achieve solutions or homogenous suspensions suitable for po administration.

Formulations were administered to 3 male C57BL/6 mice at a volume of 10 mL/kg resulting in a dose level of 10 mg/kg. Blood samples were collected at 1 and 2 hours post dose into tubes containing K2EDTA as anticoagulant, processed to plasma and stored at −60° C. or lower until LC-MS/MS analysis. Brains were harvested 2 hours post dose, rinsed with saline, dried, weighed and homogenised under ice cold conditions. Brain homogenates were stored at −60° C. or lower until LC-MS/MS analysis.

Dose formulation concentrations were verified using a LC-UV or LC-MS/MS method. Test compound concentrations in plasma and brain homogenate were quantitatively determined using LC-MS/MS methods developed in individual matrices against calibration curves with QC samples included and acceptance criteria as per CRO SOPs. Concentrations in brain homogenate were corrected for the dilution factor used to prepare the homogenate to give concentrations in whole brain tissue.

Plasma and brain concentration versus time data were reported and plotted in excel. The brain to plasma ratio at 2 hours post dose was calculated for each animal using the following equation:

Brain:plasma=brain concentration (ng/g) at 2 h/plasma concentration (ng/mL) at 2 h

As only unbound test compound is available to exert an effect on the target, unbound plasma (Cp,ᵤ) and unbound brain (Cb,ᵤ) concentrations were calculated by correcting the total concentrations for the unbound fraction in plasma (fu,ₚ) or brain (fu,_{b}) determined from in vitro plasma protein or brain tissue binding assays using the following equations:

$$Cp , u = plasma concentration ⋆ fu , p$$

$$Cb , u = brain concentration ⋆ fu , b$$

The unbound partitioning coefficient (Kp_{u,u}) was then calculated based on the ratio of Cb,ᵤ to Cp,ᵤ using the following equation:

$$K {p}_{u , u} = C {b}_{, u} / {Cp}_{, u}$$

Selected data from pharmacokinetic studies are summarized in Table 3. In these studies, mice were orally administrated a 10 mg/kg dose and sacrificed 2 hours post dose and tissue samples (brain homogenate and plasma) were analyzed subsequently as described above.

TABLE 3

| | total | free | total | free | |
|---|---|---|---|---|---|
| | plasma | plasma | brain | brain | |
| | conc. | conc. | conc. | conc. | |
| Example | (nM) | (nM) | (nM) | (nM) | Kp_{u,u} |
| 1 | 21941 | 2852 | 5512 | 3801 | 1.33 |
| 2 | 9366 | 2126 | 2189 | 1388 | 0.65 |
| 3 | 6390 | 469 | 1660 | 224 | 0.48 |
| 10 | 15087 | 1610 | 5196 | 507 | 0.32 |
| 17 | 1680 | 115 | 77 | 10 | 0.08 |
| 18 | 13000 | 3292 | 430 | 122 | 0.03 |
| 21 | 11899 | 494 | 5467 | 197 | 0.40 |
| 29 | 11082 | 1255 | 1774 | 224 | 0.18 |

The data in Table 3 illustrates that N-(4-aminocyclohexyl)pyrimidine-4-carboxamides such as Examples 1-3, 10 and 21 display excellent brain permeability, whereas similar cyclohexyl ethers or alcohols such as Comparative Examples 17-18 and similar pyrrolopyrimidines such as Comparative Example 29 (included for illustration) have much reduced brain exposure and lower Kp_{u,u} values. This is a surprising observation, particularly since the additional hydrogen bond donors in Examples 1-3 and 21 compared to Comparative Examples 17-18 would be expected to reduce brain exposure.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 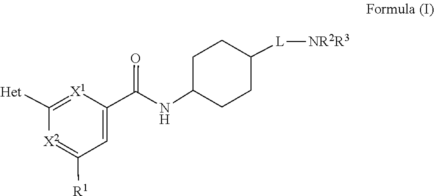
or a pharmaceutically acceptable salt, solvate or prodrug thereof, wherein:
Het is a 5-membered heteroaryl group comprising two heteroatoms independently selected from N and S, wherein the 5-membered heteroaryl group may optionally be substituted with one or two substituents independently selected from C₁-C₃ alkyl, C₁-C₃ fluoroalkyl and C₁-C₃ hydroxyalkyl;
X¹ is CH or N, and X² is CH or N, wherein at least one of X¹ and X² is N;
L is a bond, CH₂, CHMe, CMe₂ or CO;
R¹ is C₁-C₄ alkyl, C₃-C₆ cycloalkyl, hydroxyl, —O—(C₁-C₄ alkyl), or —O—(C₃-C₆ cycloalkyl), each of which may optionally be fluoro-substituted;
R² is hydrogen, C₁-C₄ alkyl, C₁-C₄ fluoroalkyl, —CHO, —CO—(C₁-C₃ alkyl) or —CO—(C₁-C₃ fluoroalkyl);
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

2. The compound, salt, solvate or prodrug as claimed in claim 1, wherein L is a bond, CH₂ or CO.

3. The compound, salt, solvate or prodrug as claimed in claim 1, wherein the compound is of formula (II): 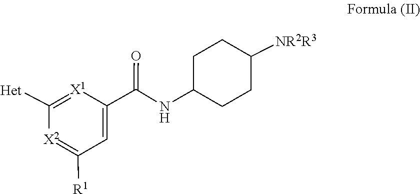
or a pharmaceutically acceptable salt, solvate or prodrug thereof, wherein:
Het is a 5-membered heteroaryl group comprising two heteroatoms independently selected from N and S, wherein the 5-membered heteroaryl group may optionally be substituted with one or two substituents independently selected from C₁-C₃ alkyl, C₁-C₃ fluoroalkyl and C₁-C₃ hydroxyalkyl;
X¹ is CH or N, and X² is CH or N, wherein at least one of X¹ and X² is N;
R¹ is C₁-C₄ alkyl, C₃-C₆ cycloalkyl, hydroxyl, —O—(C₁-C₄ alkyl), or —O—(C₃-C₆ cycloalkyl), each of which may optionally be fluoro-substituted;
R² is C₁-C₃ alkyl or C₁-C₃ fluoroalkyl;
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

4. The compound, salt, solvate or prodrug as claimed in claim 1, wherein X¹ is N and X² is N.

5. The compound, salt, solvate or prodrug as claimed in claim 1, wherein the compound is of formula (III): 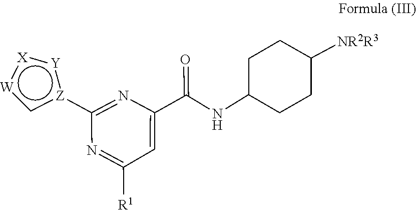
or a pharmaceutically acceptable salt, solvate or prodrug thereof, wherein:
each W, X, Y and Z is independently CH, CMe, N, NH, NMe or S, wherein two of W, X, Y and Z are CH or CMe, and the other two of W, X, Y and Z are N, NH, NMe or S;
R¹ is C₁-C₄ alkyl or C₃-C₄ cycloalkyl;
R² is C₁-C₃ alkyl or C₁-C₃ fluoroalkyl;
R³ is hydrogen or methyl; or
R² and R³, together with the nitrogen to which they are attached, form an azetidin-1-yl, pyrrolidin-1-yl or piperidin-1-yl group, each of which may optionally be fluoro-substituted.

6. The compound, salt, solvate or prodrug as claimed in claim 1, wherein Het or the group 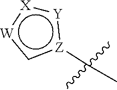
is an imidazol-1-yl, 1-methyl-imidazol-5-yl, pyrazol-4-yl, or thiazol-5-yl group.

7. The compound, salt, solvate or prodrug as claimed in claim 1, wherein R¹ is methyl, ethyl, n-propyl, iso-propyl or cyclopropyl.

8. The compound, salt, solvate or prodrug as claimed in claim 1, wherein R² is C₁-C₃ fluoroalkyl.

9. The compound, salt, solvate or prodrug as claimed in claim 1, wherein R² and R³, together with the nitrogen to which they are attached, form an azetidin-1-yl, pyrrolidin-1-yl or piperidin-1-yl group, each of which may optionally be substituted with one, two, three or four fluoro substituents.

10. The compound, salt, solvate or prodrug as claimed in claim 1, wherein the two substituents on the cyclohexyl group are trans to each other.

11. The compound, salt, solvate or prodrug as claimed in claim 1, wherein the compound is selected from:
2-(1H-imidazol-1-yl)-6-methyl-N-((1r,4r)-4-((2,2,2-trifluoroethyl)amino)cyclohexyl)pyrimidine-4-carboxamide;
N-((1r,4r)-4-((2,2-difluoroethyl)amino)cyclohexyl)-2-(1H-imidazol-1-yl)-6-methyl-pyrimidine-4-carboxamide;
6-cyclopropyl-N-((1r,4r)-4-((2,2-difluoroethyl)amino)cyclohexyl)-2-(1H-imidazol-1-yl)pyrimidine-4-carboxamide;
6-methyl-2-(1-methyl-1H-imidazol-5-yl)-N-((1r,4r)-4-((2,2,2-trifluoroethyl)amino)cyclohexyl)pyrimidine-4-carboxamide;
6-methyl-2-(thiazol-5-yl)-N-((1r,4r)-4-((2,2,2-trifluoroethyl)amino)cyclohexyl)pyrimidine-4-carboxamide;
6-ethyl-2-(1H-imidazol-1-yl)-N-((1r,4r)-4-((2,2,2-trifluoroethyl)amino)cyclohexyl)pyrimidine-4-carboxamide;
2-(1H-imidazol-1-yl)-6-methyl-N-((1r,4r)-4-(methyl(2,2,2-trifluoroethyl)amino)cyclohexyl)pyrimidine-4-carboxamide;
N-((1r,4r)-4-((2,2-difluoroethyl)amino)cyclohexyl)-6-methyl-2-(thiazol-5-yl)pyrimidine-4-carboxamide;
N-((1r,4r)-4-((2-fluoroethyl)amino)cyclohexyl)-2-(1H-imidazol-1-yl)-6-methyl-pyrimidine-4-carboxamide;
N-((1r,4r)-4-(3,3-difluoropyrrolidin-1-yl)cyclohexyl)-2-(1H-imidazol-1-yl)-6-methyl-pyrimidine-4-carboxamide;
N-((1s,4s)-4-(3,3-difluoropyrrolidin-1-yl)cyclohexyl)-2-(1H-imidazol-1-yl)-6-methyl-pyrimidine-4-carboxamide;
N-((1s,4r)-4-((S)-3-fluoropyrrolidin-1-yl)cyclohexyl)-2-(1H-imidazol-1-yl)-6-methyl-pyrimidine-4-carboxamide;
N-((1r,4r)-4-((R)-3-fluoropyrrolidin-1-yl)cyclohexyl)-2-(1H-imidazol-1-yl)-6-methyl-pyrimidine-4-carboxamide;
6-methyl-2-(1H-pyrazol-4-yl)-N-((1r,4r)-4-((2,2,2-trifluoroethyl)amino)cyclohexyl)pyrimidine-4-carboxamide;
N-((1r,4r)-4-((2,2-difluoroethyl)amino)cyclohexyl)-6-methyl-2-(1H-pyrazol-4-yl)pyrimidine-4-carboxamide;
4-cyclopropyl-N-((1r,4r)-4-((2,2-difluoroethyl)amino)cyclohexyl)-6-(1H-imidazol-1-yl)picolinamide;
N-((1r,4r)-4-(ethylamino)cyclohexyl)-2-(1H-imidazol-1-yl)-6-methyl-pyrimidine-4-carboxamide;
4-cyclopropyl-6-(1H-imidazol-1-yl)-N-((1r,4r)-4-((2,2,2-trifluoroethyl)amino)cyclohexyl)picolinamide;
2-(1H-imidazol-1-yl)-6-methyl-N-((1r,4r)-4-((1,1,1-trifluoro-2-methylpropan-2-yl)amino)cyclohexyl)pyrimidine-4-carboxamide;
2-(1H-imidazol-1-yl)-6-methyl-N-((1S,4r)-4-(((S)-1,1,1-trifluoropropan-2-yl)amino)cyclohexyl)pyrimidine-4-carboxamide;
2-(1H-imidazol-1-yl)-6-methyl-N-((1R,4r)-4-(((R)-1,1,1-trifluoropropan-2-yl)amino)cyclohexyl)pyrimidine-4-carboxamide;
6-methyl-2-(5-methyl-1H-imidazol-1-yl)-N-((1r,4r)-4-((2,2,2-trifluoroethyl)amino)cyclohexyl)pyrimidine-4-carboxamide;
2-(1H-imidazol-1-yl)-6-methyl-N-((1r,4r)-4-(((2,2,2-trifluoroethyl)amino)methyl)cyclohexyl)pyrimidine-4-carboxamide;
2-(1H-imidazol-1-yl)-6-methyl-N-((1r,4r)-4-((2,2,2-trifluoroethyl)carbamoyl)cyclohexyl)pyrimidine-4-carboxamide;
N-((1r,4r)-4-aminocyclohexyl)-2-(1H-imidazol-1-yl)-6-methyl-pyrimidine-4-carboxamide;
2-(1H-imidazol-1-yl)-6-methyl-N-((1r,4r)-4-((2,2,2-trifluoroethyl-1,1-d₂)amino)cyclohexyl)pyrimidine-4-carboxamide;
or an enantiomer of any of the foregoing;
or a pharmaceutically acceptable salt, solvate or prodrug of any of the foregoing.

12. A process for the preparation of a compound, salt, solvate or prodrug as claimed in claim 1, wherein the process comprises the step of reacting a compound of formula (IV) with an amine of formula (V): 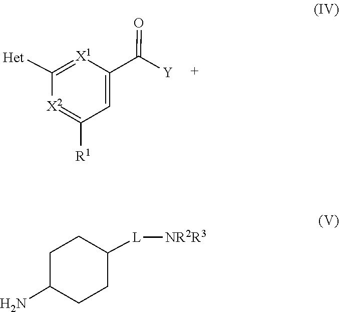
wherein Het, X¹, X², L, R¹, R² and R³ are as defined in any one of claim 1 to 11; Y is —OH, —OR⁴, —O—CO—R⁴ or —Cl; and R⁴ is C₁-C₃ alkyl;
and optionally thereafter carrying out one or more of the following procedures:
converting a compound of formula (I), (II) or (III) into another compound of formula (I), (II) or (III);
removing any protecting groups;
forming a pharmaceutically acceptable salt.

13. A pharmaceutical composition comprising a compound, salt, solvate or prodrug as claimed in claim 1, in association with a pharmaceutically acceptable adjuvant, diluent or carrier, and optionally one or more other therapeutic agents.

14. (canceled)

15. A method of treating or preventing a disease, disorder or condition associated with CD38 activity, wherein the method comprises administering a therapeutically or prophylactically effective amount of a compound, salt, solvate or prodrug as claimed in claim 1, to a patient in need thereof.

16. A method of treating or preventing a CNS disease, a disease requiring treatment via the CNS, a neurodegenerative condition, a neurological disease, an age-related disorder, or an inflammatory disorder, wherein the method comprises administering a therapeutically or prophylactically effective amount of a compound, salt, solvate or prodrug as claimed in claim 1, to a patient in need thereof.

17. A method of treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome, wherein the method comprises administering a therapeutically or prophylactically effective amount of a compound, salt, solvate or prodrug as claimed in claim 1, to a patient in need thereof.
